(19) United States
(12) Patent Application Publication
(10) Pub. No.: US 2026/0224746 A1
(43) Pub. Date: Aug. 6, 2026

(54) GENE EDITING FOR SURGERY-RELATED FIBROSIS TREATMENT

(71) Applicant: ORTHOBIO THERAPEUTICS, INC., Vail, CO (US)

(72) Inventors: Peter J. Millett, Edwards, CO (US); Matthew J. Allen, Cambridge (GB); George Gentsch, Cambridge (GB); Rahul Sharma

(21) Appl. No.: 19/149,509

(22) PCT Filed: Jan. 19, 2024
(86) PCT No.: PCT/US2024/012268

§ 371 Date: Jul. 18, 2025

Related U.S. Application Data
(60) Provisional application No. 63/480,642, filed on Jan. 19, 2023.

(30) Foreign Application Priority Data
Jan. 26, 2023 (WO) PCT/US2023/061392

(51) Int. Cl. A61K 48/00; A61K 9/1272; A61K 9/51; C12N 9/22; C12N 15/11; C12N 15/86; C12N 15/88

(52) U.S. Cl. CPC A61K 48/005; A61K 9/1272; A61K 9/5123; A61K 48/0075; C12N 9/226; C12N 15/111; C12N 15/86; C12N 15/88; C12N 2310/20; C12N 2750/14143

(57) ABSTRACT

Provided herein are compositions and methods for treating musculoskeletal fibrosis and/or scarring by ablating intracellular signaling through specific cell surface receptors through genetic editing. In some aspects, the compositions and methods are directed to the TGFB 1 ligand. In other aspects, the compositions and methods are directed to the TGFB1 receptors (TGFBR1/TGF-BR2). In some aspects, the compositions and methods are to treat or prevent post-trauma fibrosis and/or scarring. In some aspects, the compositions and methods are to treat or prevent postoperative fibrosis and/or scarring. In some aspects, the compositions and methods are for treating or preventing localized nociception, inflammation, degeneration, or morphological changes associated with fibrosis and/or scarring. In some aspect, the compositions and methods are for treating fibrosis.

### CROSS REFERENCE TO RELATED APPLICATIONS

This application claims priority to International Application No. PCT/US23/61392, filed on Jan. 26, 2023, and U.S. Provisional Patent Application No. 63/480,642, filed Jan. 19, 2023, the contents of which are hereby incorporated by reference herein, in their entireties, for all purposes.

### REFERENCE TO SEQUENCE LISTING SUBMITTED ELECTRONICALLY

This application contains a Sequence Listing which has been submitted electronically in XML file format and is hereby incorporated by reference in its entirety. The XML file, named 123994-5004-WO50 Sequence Listing.xml, was created on Jan. 17, 2024, and is 3,360 kilobytes in size.

### BACKGROUND OF THE INVENTION

Musculoskeletal tissue is particularly prone to fibrosis and/or scarring following trauma. In many instances, this trauma may be a surgical procedure. Fibrosis is the result of extracellular matrix (ECM) components accumulating at a particular site and contributes to the formation of scar tissue. See, Sheets, K., et al (2022). Journal of Cellular Biochemistry. This musculoskeletal scar tissue presents several issues, such as limiting range of motion or pain, and may even drive further disease and complicate the medical or surgical management of musculoskeletal tissue and joints.

Transforming growth factor-beta (TGFB) signals through canonical receptor-ligand interactions, which is responsible for transmission of extracellular signals across the plasma membrane of a receptor-expressing cell. In many instances, a circulating ligand will bind to a particular receptor anchored into cellular membrane, which, through activity of the receptor's cytoplasmic domain, results in the transduction of signaling pathways from the cell surface to the interior. TGFB signaling events can, in turn, impact a variety of cellular activities, which in turn effectuate changes at the tissue and organismal levels.

One such change is fibrosis of musculoskeletal tissues. Indeed, increased expression of TGFB1, among other factors, is observed in fascial fibroblasts at the site of such musculoskeletal trauma. See, Ihn, H. (2019). Allergology International, 68 (4), 437-439. This expression pattern correlated with higher expression of type I collagen and fibronectin-ECM components whose aggregation can contribute to fibrosis. A known driver of pro-inflammatory signaling, TGFB provides a link between fibrosis and/or scarring and chronic inflammation.

Current treatments for such musculoskeletal conditions, such as conservative care, physical rehabilitation, or surgery, focus on treating symptoms rather than addressing the causes of the fibrosis and/or scarring and may cause additional trauma. As such, new methods and compositions that address the causes of this condition are acutely needed.

### BRIEF SUMMARY OF THE INVENTION

Provided herein are compositions and methods for silencing the signaling functionality of either TGFB1 or its cellular receptors in an animal in need thereof to treat a disease, illness or condition caused by aberrant or excessive signaling through said receptor.

In some embodiments, receptor signaling is silenced by CRISPR editing of the gene encoding the TGFBR1 receptor. In some embodiments, receptor signaling is silenced by CRISPR editing of the gene encoding the TGFBR2 receptor. In some embodiments, receptor signaling is silenced by CRISPR editing of the gene encoding the TGFB 1 ligand. In some embodiments, the CRISPR editing results in ablation of a transmembrane domain (i.e., generation of soluble receptor). In some embodiments, the CRISPR editing results in ablation of a cytoplasmic domain.

The ubiquity of the receptor-ligand paradigm in cellular biology means that numerous diseases, illnesses, and conditions are caused, wholly or in part, by aberrant or excessive signaling through various cellular receptors, and various approaches have been utilized to address this. Small and large molecules can be used to disrupt receptor-ligand interactions, though issues of off-target effects and potential immunogenicity remain.

More recently, genetic approaches have been explored to either transiently reduce (i.e., knockdown), e.g., in the case of siRNA, or permanently ablate (i.e., genetic knockout), the expression of a given ligand or receptor. However, more advanced genetic tools are needed to address the myriad of diseases, illnesses and conditions implicated by such aberrant signaling.

Notably, compositions and methods disclosed herein also encompass various routes of administration of CRISPR components with a particular focus on post-traumatic and post-surgical interventions to treat and prevent fibrosis tissues such as musculoskeletal tissue, cardiac tissue, pulmonary tissue, kidney tissue, and the like.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIGS. 1A, 1B, 1C, 1D, 1E, IF, 1G, and 1H collectively illustrate SEQ ID NOs: 1-198, (A-D) the spacer sequences of CRISPR RNA (crRNA) generated by the bioinformatic methods herein described that targets CRISPR/Cas9 to human TGFB1 to generate a genetic knockout and (E-H) additional information regarding the genomic coordinates of the bound DNA, DNA strand targeted, exon targeted, and several quality control parameters.

FIGS. 2A, 2B, 2C, 2D, 2E, and 2F collectively illustrate SEQ ID NOs: 199-320, (A-C) the spacer sequences of crRNA generated by the bioinformatic methods herein described that targets CRISPR/Cas9 to human TGFBR1 to generate genetic knockouts, soluble or membrane-bound decoy receptors and (D-F) additional information regarding the genomic coordinates of the bound DNA, DNA strand targeted, exon targeted, and several quality control parameters.

FIGS. 3A, 3B, 3C, 3D, 3E, 3F, 3G, and 3H collectively illustrate SEQ ID NOs: 321-519, (A-D) the spacer sequences of crRNA generated by the bioinformatic methods herein described that targets CRISPR/Cas9 to human TGFBR2 to generate genetic knockouts, soluble or membrane-bound decoy receptors and (E-H) additional information regarding the genomic coordinates of the bound DNA, DNA strand targeted, exon targeted, and several quality control parameters.

FIGS. 4A and 4B illustrate (A) a schematic view showing the targeted domains of TGFBR1 and the orientation of various spacer sequences of Cas9-recruiting single guide RNAs (sgRNAs) predicted to generate knockouts and (B) a set of parameters considered for designing sgRNAs against TGFBR1, as well as example sgRNA-targeted protospacer DNA sequences.

FIGS. 5A and 5B illustrate (A) a schematic view showing the targeted domains of TGFBR1 and the orientation of various protospacer sequences targeted by CRISPR/Cas9 predicted to generate knockouts, soluble or membrane-bound decoy receptors (ECD: Extracellular Domain; TMD: Transmembrane Domain; ICD: Intracellular Domain; GSM: GS rich Motif); and (B) a set of parameters considered for designing sgRNAs against TGFBR1, as well as example sgRNA-targeted protospacer DNA sequences.

FIGS. 6A and 6B illustrate (A) a schematic view showing the targeted domains of TGFBR2 and the orientation of various protospacer sequences targeted by CRISPR/Cas9 predicted to generate knockouts and soluble or membrane-bound decoy receptors (ECD: Extracellular Domain; TMD: Transmembrane Domain; ICD: Intracellular Domain; GSM: GS rich Motif); and (B) a set of parameters considered for designing sgRNAs against TGFBR2, as well as example sgRNA-targeted protospacer DNA sequences.

FIGS. 7A and 7B show a summary of the efficiency for editing the human TGFBR1 gene in THP-1 cells using the identified sgRNAs and (A) wild type Cas9 (WTCas9) or (B) high-fidelity (R691A) Cas9 (ARCas9) protein. The bar graph shows both top and all other frameshift edits contributing to the knock-out (KO) score as inferred by Sanger trace deconvolution.

FIGS. 8A and 8B show a summary of the efficiency for top editing the human TGFBR1 gene in THP-1 cells using the identified sgRNAs and (A) wild type Cas9 (WTCas9) or (B) high-fidelity (R691A) Cas9 (ARCas9) protein. The bar graph shows both top and all other frameshift edits contributing to the knock-out (KO) score as inferred by Sanger trace deconvolution.

FIGS. 9A and 9B show a summary of the efficiency for editing the human TGFBR2 gene in THP-1 cells using the identified sgRNAs and (A) wild type Cas9 (WTCas9) or (B) high-fidelity (R691A) Cas9 (ARCas9) protein. The bar graph shows both top and all other frameshift edits contributing to the knock-out (KO) score as inferred by Sanger trace deconvolution.

FIGS. 10A, 10B, 10C, and 10D illustrate relative levels of (A, C) TGFB1 and (B, D) TIMP1 gene expression in THP-1 cells that are unedited (WT) or edited to knock out TGFB1 (OHTG), TGFBR1 (OHTIR), or TGFBR2 (OHTIIR) following challenge by lipopolysaccharides (LPS) (panels A and B) or TGF-beta (panels C and D) for 6 hours.

FIGS. 11A, 11B, 11C, and 11D illustrate relative levels of (A) SERPINE1 (B) Col1A2, (C) FN1, and (D) CTGF gene expression in THP-1 cells that are unedited (WT) or edited to knock out TGFB1 (OHTG), TGFBR1 (OHTIR), or TGFBR2 (OHTIIR) following challenge by TGF-beta treated for 6 hours.

FIGS. 12A, 12B, and 12C collectively illustrate the spacer sequences of crRNA targeting human (A) TGFB1 (SEQ ID NOs: 520-527), (B) TGFBR1 (SEQ ID NOs: 528-552), and (C) TGFBR2 (SEQ ID NOs: 553-604) for use in sgRNAs to validate in vitro editing with different modes of delivery.

### DETAILED DESCRIPTION OF THE INVENTION

### I. Introduction

Provided herein are compositions and methods for silencing the signaling functionality of one or more cellular receptors in an animal in need thereof to treat a disease, illness or condition caused by aberrant or excessive signaling through said receptor.

In some embodiments, receptor signaling is reduced or eliminated by use of the compositions and methods described herein to edit a TGFB1, TGFBR1, and/or TGFBR2 gene. In some embodiments, the editing knocks out the TGFB1, TGFBR1, and/or TGFBR2 gene editing of the gene such that it no longer encodes for a functional protein. In some embodiments, the editing results in ablation of a transmembrane domain of the protein encoded by the TGRBR1 or TGFBR2 gene, e.g., generation of a soluble receptor decoy. In some embodiments, the editing results in ablation of a cytoplasmic domain of the protein encoded by the TGRBR1 or TGFBR2 gene, e.g., generation of a membrane-bound receptor decoy. In some embodiments, TGFB1, TGFBR1, and/or TGFBR2 are modulated in expression by altering coding or non-coding gene regulatory sequences or their epigenetic landscape. In some embodiments, the receptor is TGFBR1. In some embodiments, the receptor is TGFBR2. In some embodiments, receptor signaling is reduced or eliminated by use of the compositions and methods described herein to edit the TGFB 1 ligand.

### II. Definitions

Unless defined otherwise, all technical and scientific terms used herein have the same meaning as is commonly understood by one of skill in the art to which this invention belongs. All patents and publications referred to herein are incorporated by reference in their entireties.

The terms “Transforming Growth Factor Beta 1” or “TGFB1” refer to the genes (NCBI Gene ID: 7040 [human], NCBI Gene ID: 403998 [canine], NCBI Gene ID: 100033900 [equine], NCBI Gene ID: 768263 [feline]) or an encoded gene product (e.g., UniProt: P01137; AAH00125 . . . 1 [human], XP_038512824.1 [canine], XP_014716505.1 [equine], XP_006941294.1 [feline]), as well as sequence variants, proteins harboring conservative amino acid substitutions, and glycoforms thereof. Canonically, the cytokine proteins encoded by the genes listed above are capable of binding the TGFBR complex to mediate intracellular signaling pathways regulating multiple physiological and pathological processes-including inflammatory processes-through release of SMAD2, which can then translocate to the nucleus or activation of other cytoplasmic signaling mediators. In some instances, and merely for the sake of disambiguation, a prefix is added when referring to the protein or gene of a particular species (with h, c, e, and f, referring to the human, canine, equine, and feline forms, respectively).

In certain embodiments, any region of a TGFB1 gene (e.g., 5′ untranslated region [UTR], exon 1, exon 2, exon 3, exon 4, exon 5, exon 6, exon 7, any intervening intronic regions, intron/exon junctions, the 3′ UTR, or polyadenylation signal) is targeted by an RNA-guided nuclease to alter the gene. In some embodiments, the TGFB1 gene targeted by an RNA-guided nuclease is from a mammal. In some embodiments, the TGFB1 gene targeted by an RNA-guided nuclease is from a human (hTGFB1). In some embodiments, the TGFB1 gene targeted by an RNA-guided nuclease is from a dog (cTGFB1). In some embodiments, the TGFB 1 gene targeted by an RNA-guided nuclease is from a horse (eTGFB1). In some embodiments, the TGFB1 gene targeted by an RNA-guided nuclease is from a cat (fTGFB1).

The terms “Transforming Growth Factor Beta Receptor 1” or “TGFBR1” refer to the genes (NCBI Gene ID: 7046 [human], NCBI Gene ID: 481628 [canine], NCBI Gene ID: 100034117 [equine], NCBI Gene ID: 101094057 [feline]) or an encoded gene product (e.g., UniProt: P36897; NP_004603.1 [human], XP_038538191.1 [canine], XP_023485510.1 [equine], XP_023098269.1 [feline]), as well as sequence variants, proteins harboring conservative amino acid substitutions, and glycoforms thereof. Canonically, the proteins encoded by the genes listed above are transmembrane serine/threonine kinases forming, with TGFBR2, the native receptor for the TGF-beta cytokines TGFB1, TGFB2, and TGFB3. When bound to its ligand, TGFBR1 is phosphorylated by TGFBR2, activating intracellular signaling regulating multiple physiological and pathological processes through release of SMAD2, which can then translocate to the nucleus or activation of other cytoplasmic signaling mediators. In some instances, and merely for the sake of disambiguation, a prefix is added when referring to the protein or gene of a particular species (with h, c, e, and f, referring to the human, canine, equine, and feline forms, respectively).

In certain embodiments, any region of an TGFBR1 gene (e.g., 5′ untranslated region [UTR], exon 1, exon 2, exon 3, exon 4, exon 5, exon 6, exon 7, exon 8, exon 9, exon 10, exon 11, exon 12, any intervening intronic regions, intron/exon junctions, the 3′ UTR, or polyadenylation signal) is targeted by an RNA-guided nuclease to alter the gene. In some embodiments, the TGFBR1 gene targeted by an RNA-guided nuclease is from a mammal. In some embodiments, the TGFBR1 gene targeted by an RNA-guided nuclease is from a human (hTGFBR1). In some embodiments, the TGFBR1 gene targeted by an RNA-guided nuclease is from a dog (cTGFBR1). In some embodiments, the TGFBR1 gene targeted by an RNA-guided nuclease is from a horse (eTGFBR1). In some embodiments, the TGFBR1 gene targeted by an RNA-guided nuclease is from a cat (fTGFBR1).

The term terms “Transforming Growth Factor Beta Receptor 2” or “TGFBR2” refer to the genes (NCBI Gene ID: 7048 [human], NCBI Gene ID: 477039 [canine], NCBI Gene ID: 100033860 [equine], NCBI Gene ID: 101091725 [feline]) or an encoded gene product (e.g., UniProt: P37173; NP_003233.4 [human], XP_038288013.1 [canine], XP_023475502.1 [equine], XP_023116415.1 [feline]), as well as sequence variants, proteins harboring conservative amino acid substitutions, and glycoforms thereof. Canonically, the proteins encoded by the genes listed above are transmembrane serine/threonine kinases forming, with TGFBR2, the native receptor for the TGF-beta cytokines TGFB1, TGFB2, and TGFB3. When bound to its ligand, TGFBR1 is phosphorylated by TGFBR2, activating intracellular signaling regulating multiple physiological and pathological processes through release of SMAD2, which can then translocate to the nucleus or activation of other cytoplasmic signaling mediators. In some instances, and merely for the sake of disambiguation, a prefix is added when referring to the protein or gene of a particular species (with h, c, e, and f, referring to the human, canine, equine, and feline forms, respectively). In some instances, and merely for the sake of disambiguation, a prefix is added when referring to the protein or gene of a particular species (with h, c, e, and f, referring to the human, canine, equine, and feline forms, respectively).

In certain embodiments, any region of an TGFBR2 gene (e.g., 5′ untranslated region [UTR], exon 1, exon 2, exon 3, exon 4, exon 5, exon 6, exon 7, exon 8, exon 9, exon 10, any intervening intronic regions, intron/exon junctions, the 3′ UTR, or polyadenylation signal) is targeted by an RNA-guided nuclease to alter the gene. In some embodiments, the TGFBR2 gene targeted by an RNA-guided nuclease is from a mammal. In some embodiments, the TGFBR2 gene targeted by an RNA-guided nuclease is from a human (hTGFBR2). In some embodiments, the TGFBR2 gene targeted by an RNA-guided nuclease is from a dog (cTGFBR2). In some embodiments, the TGFBR2 gene targeted by an RNA-guided nuclease is from a horse (eTGFBR2). In some embodiments, the TGFBR2 gene targeted by an RNA-guided nuclease is from a cat (fTGFBR2).

The term “treatment” refers to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. For example, a composition, method, or system of the present disclosure may be administered as a prophylactic treatment to a subject that has a predisposition for a given condition (e.g., arthritis). “Treatment”, as used herein, covers any treatment of a disease in a mammal, particularly in a human, canine, feline, or equine, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms.

“Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine. It is understood that compositions and methods of the present disclosure are applicable to treat all mammals, including, but not limited to human, canine, feline, equine, and bovine subjects.

The term “therapeutically effective” refers to the amount of a composition or combination of compositions as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compositions chosen, the dosing regimen to be followed, whether the composition is administered in combination with other compositions or compounds, timing of administration, the tissue to which it is administered, and the physical delivery system in which the composition is carried.

The terms “fibrosis” and “fibrotic scarring” are used interchangeably and refer to formation of an abnormal amount of fibrous tissue in an organ or tissue, typically occurring due to inflammation, irritation, or healing of the tissue. Fibrosis encompasses, the clinical phenomenona commonly referred to as scarring, e.g., tissue scarring or organ scarring, and adhesion, e.g., fibrosis formed between organs or different tissues. Fibrosis can occur in any tissue, including but not limited to joint tissues (e.g., knee joints, shoulder joints—e.g., adhesive capsulitis-elbow joints, etc.), other musculoskeletal tissues (e.g., tendon scarring), kidney tissue (e.g., in chronic kidney disease, etc.), skin tissue (e.g., associated with wound healing—e.g., following trauma or postoperative-keloid disorder, nephrogenic systemic fibrosis, scleroderma/systemic sclerosis in humans, “proud flesh” in horses, etc.), lung tissue (e.g., in fibrothorax, pulmonary fibrosis, cystic fibrosis, idiopathic pulmonary fibrosis, radiation-induced lung injury, progressive massive fibrosis, scleroderma/systemic sclerosis, etc.), liver tissue (e.g., in cirrhosis, bridging fibrosis, etc.),, cardiac tissues (e.g., interstitial fibrosis-typically associated with congestive heart failure, hypertension, and normal aging-replacement fibrosis-typically associated with prior myocardial infarction-etc.), brain tissue (e.g., glial scars, etc.), intestinal tissue (e.g., in Crohn's disease, etc.), hands and/or fingers (e.g., in Dupuytren's contracture, etc.), lymph tissue (e.g., mediastinal fibrosis, etc.), bone marrow tissue (e.g., myelofibrosis, etc.), penile tissue (e.g., in Peyronie's disease, etc.) soft tissue of the retroperitoneum (e.g., retroperitoneal fibrosis, etc.), musculoskeletal tissue, etc.

In some embodiments, fibrosis results from trauma to the tissue. In some embodiments, fibrosis is associated with a surgical procedure, e.g., postoperative fibrosis. Non-limiting examples of procedures that may induce fibrosis include ligament reconstruction, anterior cruciate ligament (ACL) reconstruction, autograft ACL reconstruction, allograft ACL reconstruction, fracture repair, total knee arthroplasty (TKA), and microdiscectomy. Additionally, the fibrosis may be caused by any of the following conditions, which may be induced or exacerbated by a surgical procedure: knee arthrofibrosis, intra-articular fibrous nodules, or epidural fibrosis.

The term “musculoskeletal trauma,” as used herein, pertains to any injury that affects the bones, muscles, ligaments, nerves, or tendons. In some embodiments, the trauma is the result of a surgical procedure

The terms “polynucleotide,” “nucleotide,” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, IRNA, tRNA, lncRNA, RNA antagomirs, and inhibitory DNA or RNA (RNAi, e.g., small or short hairpin (sh) RNA, microRNA (miRNA), aptamers, small or short interfering (si) RNA, trans-splicing RNA, or antisense RNA). Polynucleotides also include non-coding RNA, which include for example, but are not limited to, RNAi, miRNAs, lncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

The term “gene” or “nucleotide sequence encoding a polypeptide” refers to the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons). For example, a gene includes a polynucleotide containing at least one open reading frame capable of encoding a particular protein or polypeptide after being transcribed and translated.

The term “extracellular domain,” as it refers to transmembrane cellular receptors, is defined as the portion of the protein that is exposed to the extracellular environment and is able to engage with and/or bind a ligand.

The terms “cytoplasmic domain” and “intracellular domain” may be used interchangeably and, when referring to transmembrane receptors, define the portion of the protein this is exposed to the cytoplasm. In many instances, these portions of the proteins comprise signaling domains to recruit and associate with various intracellular factors. Following engagement with a ligand via the extracellular domain, the interaction effects changes that may result in new association, dissociation or recruitment of various cytoplasmic factors that aid in transducing a signal.

The term “transmembrane domain,” as it refers to transmembrane receptors, is defined as the portion of the protein is embedded within the plasma membrane (i.e., not exposed to either the extracellular environment or the cytosol). Transmembrane domains are generally of a more hydrophobic character than either the extracellular or cytoplasmic portions and often adopt higher order helical structures. Though its primary role is an anchor, ligand-induced conformational changes to particular receptors have been shown to impact the transmembrane domain such that it is integral to the subsequent intracellular signaling.

The term “receptor” refers to a protein capable of binding another cognate protein (i.e., its ligand) with high affinity. This receptor-ligand interaction may be 1:1, or result in multimerization, wherein numerous proteins aggregate to bind one or more ligands. Receptors are generally present at the cell surface, such that they may most efficiently encounter a ligand and initiate intracellular signaling.

The term “intracellular signaling” refers to cellular changes that result due to events occurring at the cell surface. Typically, a soluble ligand binds its receptor at the cell surface, which can induce changes in the receptor, such that associated intracellular factors are also affected. These factors may then impact others within the cell, and this cascade continues until, in many cases, a particular factor is able to alter gene expression in the nucleus in response to the stimulus at the surface.

The term “gene regulatory sequence” refers to double-stranded DNA sequences in the genome that control the level of gene transcription.

The term “RNA-guided nuclease” refers to an enzyme capable of finding and breaking the backbone of, for example, a DNA molecule. The activity of RNA-guided nucleases is directed by a nucleic acid molecule (i.e., guide RNA). Once properly oriented to form a functional ribonucleoprotein complex, the enzyme locates a specific position within a target nucleic acid (e.g., a gene or locus) via sequence complementarity with a portion of the gRNA. Non-exhaustive examples of RNA-guided nucleases include Cas9, Cpf1, and Cas12.

The term “Cas9” refers to an RNA-guided, double-stranded DNA-binding nuclease protein, nickase protein or enzymatically inactive protein, or a variant thereof and may be used to refer to either naturally occurring or recombinant Cas9 nuclease variants (e.g., ES-Cas9, HF-Cas9, PE-Cas9, and AR-Cas9). Cas9 may be further tethered to other functional protein domains such as those of nucleobase deaminases, reverse transcriptases, epigenetic modifiers or transcriptional effectors. The wildtype Cas9 nuclease has two functional nuclease domains, e.g., RuvC and HNH, that simultaneously cut different strands of double-stranded DNA, resulting in a double-strand break. Cas9 enzymes described herein may comprise a HNH or HNH-like nuclease domain and/or a RuvC or RuvC-like nuclease domain without impacts on the ability to induce double-strand breaks in genomic DNA (e.g., at a target locus) when both functional domains are active. The Cas9 enzyme may comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

As used herein, “PAM” refers to a Protospacer Adjacent Motif and is necessary for an RNA-guided nuclease to bind a target nucleic acid. In many instances, the PAM directly abuts the complementary sequence in the target. Naturally-occurring Cas9 molecules, for example, recognize specific PAM sequences (see, e.g., Table 1). In some embodiments, a Cas9 molecule has the same PAM specificities as a naturally occurring Cas9 molecule. In other embodiments, a Cas9 molecule has a PAM specificity not associated with a naturally occurring Cas9 molecule. In other embodiments, a Cas9 molecule's PAM specificity is not associated with the naturally occurring Cas9 molecule to which it has the closest sequence homology. For example, a naturally occurring Cas9 molecule can be altered such that the PAM sequence recognition is altered to decrease off target sites, improve specificity, or eliminate a PAM recognition requirement. In an embodiment, a Cas9 molecule may be altered (e.g., to lengthen a PAM recognition sequence, improve Cas9 specificity to high level of identity, to decrease off target sites, and/or increase specificity). In an embodiment, the length of the PAM recognition sequence is at least 4, 5, 6, 7, 8, 9, 10 or 15 amino acids in length. In some embodiments, a Cas9 molecule may be altered to ablate PAM recognition.

The terms “guide RNA,” “gRNA” or “sgRNA” may be used interchangeably and refer to an RNA molecule, preferably a synthetic single guide RNA molecule, composed of a targeting spacer and scaffold sequence. These molecules, once loaded onto a functional RNA-guided nuclease can direct sequence-specific cleavage of a target nucleic acid.

An sgRNA can be administered or formulated, e.g., as a synthetic RNA, or as a nucleic acid comprising a sequence encoding the gRNA, which is then expressed in the target cells. As would be evident to one of ordinary skill in the art, various tools may be used in the design and/or optimization of an sgRNA in order to, for example, increase specificity and/or precision of genomic editing at a particular site.

In general, candidate sgRNAs may be designed and identified by first locating suitable PAMs within a genomic sequence. Then additional calculations may be utilized to predict on-target and off-target efficiencies. Available web-based tools to aid in the initial design and modeling of candidate sgRNAs include, without limitation, CALITAS, SWOffinder, CRISPR seek, CRISPR Design Tool, Cas-OFFinder, E-CRISP, ChopChop, CasOT, CRISPR direct, CRISPOR, BREAKING-CAS, CrispRGold, and CCTop. See, e.g., Safari, et al. Current Pharma. Biotechol. (2017) 18 (13), which is incorporated by reference herein in its entirety for all purposes. Such tools are also described, for example, in PCT Publication No. WO2014093701A1 and Liu, et al., “Computational approached for effective CRISPR guide RNA design and evaluation”, Comput Struct Biotechnol J., 2020; 18:35-44, each of which is incorporated by reference herein in its entirety for all purposes. Candidate sgRNAs may be further assessed experimental screening or other methodologies.

The terms “spacer” refer to the portion of an sgRNA molecule with complementarity to the target nucleic acid. Canonically, the spacer is a portion of a CRISPR RNA (crRNA) that hybridizes to the target nucleic acid, e.g., via the spacer sequence, and hybridizes with a trans-activating crispr RNA (tracrRNA), e.g., via a non-targeting sequence. However, when crRNA is used herein in conjunction with a sequence targeting a gene, e.g., a TNFB or TNFBR gene, the sequence being referred to is the spacer sequence, as opposed to both the spacer and non-targeting sequences present in a canonical crRNA molecule. In an sgRNA, the crRNA and tracrRNA are combined into a single molecule.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that compositions, amounts, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80%, 90%, 95%, 96%, 97%, 98%, 99%, 99.5%, 99.9%, 99.99%, or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed methods and compositions. All compositions, methods, and kits described herein that embody the present disclosure can, in alternate embodiments, be more specifically defined by any of the transitional terms “comprising,” “consisting essentially of,” and “consisting of.”

### III. Methods

A. CRISPR

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In one aspect, the present disclosure encompasses compositions relating to clustered regularly interspaced short palindromic repeats (CRISPR) and CRISPR-associated RNA-guided nucleases and associated methods, components, and compositions (hereafter, CRISPR/Cas systems). Such systems minimally require at least one isolated or non-naturally-occurring RNA-guided nuclease (e.g., a Cas9 protein) and at least one isolated or non-naturally-occurring guide RNA (e.g., an sgRNA) to effectuate augmentation of a nucleic acid sequence (e.g., genomic DNA) or its epigenetic features (e.g., DNA methylation).

In some embodiments, a CRISPR/Cas system effectuates the alteration of a targeted gene or locus in a eukaryotic cell by effecting an alteration of the sequence at a target position (e.g., by creating an insertion or deletion (collectively, an indel) resulting in loss-of-function of (i.e., knocking out) the affected gene or allele; e.g., a nucleotide substitution resulting in a truncation, nonsense mutation, or other type of loss-of-function of an encoded product of, for example, one or more TGFB1, TGFBR1 or TGFBR2 gene (i.e., mRNA or protein); a deletion of one or more nucleotides resulting in a truncation, nonsense mutation, or other type of loss-of-function of an encoded product of, for example, one or more TGFB1, TGFBR1 or TGFBR2 gene; e.g., loss-of-function of the encoded mRNA or protein by a single nucleotide, double nucleotide, or other frame-shifting deletion, or a deletion resulting in a premature stop codon; or an insertion resulting in a truncation, nonsense mutation, or other type of loss-of-function of an encoded gene product, such as an encoded gene product of, for example, one or more TGFB1, TGFBR1 or TGFBR2 gene (i.e., mRNA or protein);

e.g., a single nucleotide, double nucleotide, or other frame-shifting insertions, or an insertion resulting in a premature stop codon. In some embodiments, a CRISPR/Cas system of the present disclosure provides for the alteration of a gene and/or encoded product of a gene, such that the altered product has a resultant loss-of-function and becomes a dominant negative or decoy (e.g., a transmembrane receptor incapable of initiating intracellular signaling or a soluble receptor). In some embodiments, a CRISPR/Cas system of the present disclosure changes the transcription level of one or more TGFB1, TGFBR1 or TGFBR2 gene.

In one aspect, CRISPR/Cas systems effectuate changes to the sequence of a nucleic acid through nuclease activity. For example, in the case of genomic DNA, the RNA-guided-nuclease locates a target position within a targeted gene or locus by sequence complementarity with the target genomic sequence (e.g., spacer sequence of the CRISPR RNA (crRNA) or synthetic single guide RNA (sgRNA))-cleaves the genomic DNA upon recognition of a particular, nuclease-specific motif called the protospacer adjacent motif (PAM). See generally, Collias, D., & Beisel, C. L. (2021). Nature Communications, 12 (1), 1-12.

Nuclease activity (i.e., cleavage) induces a double-strand break (DSB) in the case of genomic DNA. Endogenous cellular mechanisms of DSB repair, namely non-homologous end joining (NHEJ), microhomology-mediated end joining (MMEJ), and homologous recombination, result in erroneous repair at a given target position with some calculable frequency as a result of interference from said components of the CRISPR/Cas system, thereby introducing substitutions or indels into the genomic DNA. See generally Scully, R., et al. (2019). Nature Reviews Molecular Cell Biology, 20 (11), 698-714. At some frequency, these indels and/or substitutions may result in frameshifts, nonsense mutations (i.e., early stop codons) or truncations that impact the availability of gene products, such as mRNA and/or protein. In certain embodiments, the CRISPR/Cas system may induce a homology-directed repair (HDR) mechanism leading to insertions of non-random sequences at a target position through the use of templates (e.g., an HDR template) provided to the cell as part of the system along with the nuclease and gRNA. See Bloh, K., & Rivera-Torres, N. (2021). International Journal of Molecular Sciences, 22 (8), 3834.

In general, the minimum requirements of the CRISPR/Cas system will be dependent upon the nuclease (i.e., Cas protein) provided therewith. To this extent, these bacterially-derived nucleases have been functionally divided into Types I, III, and V, which all fall into Class 1 and Types II, IV, and VI that are grouped into Class 2.

### Class 1 CRISPR/Cas Systems:

The exact components, compositions, and methods for effectuating a change in a targeted nucleic acid sequence using a Class 1 CRISPR/Cas system will vary but should minimally include: a nuclease (e.g., selected from at least Types I, and III), at least one guide RNA, e.g., selected from 1) sgRNA or 2) a combination of crRNA and tracrRNA. These CRISPR/Cas systems have been categorized together as Class 1 CRISPR/Cas systems due to their similarities in requirements and mode of action within a eukaryotic cell. To this end, compositions, components, and methods among Class 1 constituents may be considered functionally interchangeable, and the following details, provided merely for exemplary purposes, do not represent an exhaustive list of class members:

Cas3 (see Table 1) is the prototypical Type I DNA nuclease that functions as the effector protein as part of a larger complex (the Cascade complex comprising Csel, Cse2), that is capable of genome editing. See generally He, L., et al. (2020). Genes, 11 (2), 208. Unlike other CRISPR/Cas systems, Type I systems localize to the DNA target without the Cas3 nuclease via the Cascade complex, which then recruits Cas3 to cleave DNA upon binding and locating the 3′ PAM. The Cascade complex is also responsible for processing crRNAs such that they can be used to guide it to the target position. Because of this functionality, Cascade has the ability to process multiple arrayed crRNAs from a single molecule. See Luo, M. (2015). Nucleic Acids Research, 43 (1), 674-681. As such, Type I system may be used to edit multiple targeted genes or loci from a single molecule.

Because the natural Cas3 substrate is ssDNA, its function in genomic editing is thought to be as a nickase; however, when targeted in tandem, the resulting edit is a result of blunt end cuts to opposing strands to approximate a blunt-cutting endonuclease, such as Cas9. See Pickar-Oliver, A., & Gersbach, C. A. (2019). Nature Reviews Molecular Cell Biology, 20 (8), 490-507.

Like Type I nucleases, the Type III system relies upon a complex of proteins to effect nucleic acid cleavage. Particularly, Cas10 possesses the nuclease activity to cleave ssDNA in prokaryotes. See Tamulaitis, G. Trends in Microbiology, 25 (1), 49-61. Interestingly, this CRISPR/Cas system, native to archea, exhibits dual specificity and targets both ssDNA and ssRNA. Aside from this change, the system functions much like Type I in that the crRNA targets an effector complex (similar to Cascade) in a sequence-dependent manner. Similarly, the effector complex processes crRNAs prior to association. The dual nature of this nuclease makes its applications to genomic editing potentially more powerful, as both genomic DNA and, in some cases, mRNAs with the same sequence may be targeted to silence particular targeted genes.

### Class 2 CRISPR/Cas Systems:

The exact components, compositions, and methods for effectuating a change in a targeted nucleic acid sequence using a Class 2 CRISPR/Cas system will vary, but should minimally include: a nuclease (selected from at least Types II, and V), at least one guide RNA selected from 1) sgRNA or 2) a combination of crRNA and tracrRNA. These CRISPR/Cas systems have been categorized together as Class 2 CRISPR/Cas systems due to their similarities in requirements and mode of action within a eukaryotic cell. To this end, compositions, components, and methods among Class 2 constituents may be considered functionally interchangeable, and the following details, provided merely for exemplary purposes, do not represent an exhaustive list of class members:

Type II nucleases are the best-characterized CRISPR/Cas systems, particularly the canonical genomic editing nuclease Cas9 (see Table 1). Multiple Cas9 proteins, derived from various bacterial species, have been isolated. The primary distinction between these nucleases is the PAM, a required recognition site within the targeted dsDNA. After association with a gRNA molecule, the spacer sequence orients the nuclease at the proper position, but the protein's recognition of the PAM is what induces a cleavage event near that site, resulting in a blunt DSB.

In addition to the naturally-derived Cas9 proteins, several engineered variants have similarly been reported. These range from Cas9 with enhanced specific (i.e., less off-target activity), such as espCas9. Others have been catalytically modified via point mutations in the RuvC (e.g., D10A) and HNH (e.g., H840A) domains such that they induce only single-strand breaks (i.e., Cas9 nickases). See Frock, R. et al. (2015). Nature Biotechnology, 33 (2), 179-186. These have also been shown to be less error-prone in editing. Such mitigation of off-target effects becomes paramount when selecting for a desired insertion (i.e., a knock in mutation, in which a desired nucleotide sequence is introduced into a target nucleic acid molecule) rather than a deletion. Indeed, less off-target effects may aid in the preferred DNA repair mechanism (HDR, in most instances for knock in mutations). See generally Naeem, M., et al. (2020). Cells, 9 (7), 1608.

In some embodiments, the CRISPR-associated endonuclease is a Cas9 endonuclease. In some embodiments, the Cas9 endonuclease comprises K848A, K1003A, and R1060A amino acid substitutions. In some aspects, the Cas9 endonuclease comprises a R691A amino acid substitution. In some embodiments, the Cas9 endonuclease comprises N497A, R661A, Q695A, and Q926A amino acid substitutions. In some embodiments, the Cas9 endonuclease comprises N692A, M694A, Q695A, and H698A amino acid substitutions. In some aspects, the Cas9 endonuclease comprises R63A and Q768A amino acid substitutions. In some aspects, the Cas9 endonuclease comprises M495V, Y515N, K526E and R661Q amino acid substitutions. In some aspects, the Cas9 endonuclease comprises K526E or K526N amino acid substitutions. In some embodiments, the Cas9 endonuclease comprises F539S, M763I, and K890N amino acid substitutions. In some embodiments, the Cas9 endonuclease comprises an E1007L amino acid substitution. In some embodiments, the Cas9 endonuclease comprises N690C, T769I, G915M and N980K amino acid substitutions. In some embodiments, the Cas9 endonuclease comprises an E1007L amino acid substitution.

Additional exemplary engineered variants of canonical Cas proteins (e.g., mutants, chimeras) include the following (each of which are hereby incorporated by reference in their entireties for all purposes): WO2015035162A2, WO2019126716A1, WO2019126774A1, WO2014093694A1, WO2014150624A1, US20190225955A1, U.S. patent Ser. No. 11/427,818, U.S. patent Ser. No. 11/242,542, U.S. patent Ser. No. 11/098,297, U.S. patent Ser. No. 10/876,100, U.S. patent Ser. No. 10/767,193, U.S. patent Ser. No. 10/494,621, and U.S. patent Ser. No. 10/100,291.

For the avoidance of doubt, Cas9 of Streptococcus pyogenes (SpCas9) collectively refers to any one of the group consisting of espCas9 (also referred to herein as ESCas9 or esCas9), HFCas9, PECas9, ARCas9.

Like the canonical Cas9 systems, Type V nucleases only require a synthetic sgRNA with a targeting domain complementary to a genomic sequence to carry out genomic editing. These nucleases contain a RuvC domain but lack the HNH domain of Type II nucleases. Further, Cas12, for example, leaves a staggered cut in the dsDNA substrate distal to the PAM, as compared to Cas9's blunt cut next to the PAM. Both Cas12a, also known as Cpf1, and Cas12b, also known as C2cl (see Table 1), act as part of larger complex of two gRNA-associated nucleases that acts on dsDNA as quaternary structure nicking each strand simultaneously. See Zetsche B, et al. Cell. 2015; 163 (3): 759-771.; see also Liu L, Chen P, Wang M, et al. Mol Cell. 2017; 65 (2): 310-322. Additionally, Cas12b (C2c1) is a highly accurate nuclease with little tolerance for mismatches. See Yang H, et al. Cell. 2016; 167 (7): 1814-1828.e12.

TABLE 1
Exemplary list of Cas nucleases and their requirements

| | | | Spacer |
|---|---|---|---|
| | PAM | Type of end generated | length |
| Nuclease (Species) | (5′→3′) | (nucleic acid target) | (nt) |
| Cas9 (S. pyogenes) | NGG | Blunt (dsDNA) | 20 |
| Cas9 (S. aureus) | NNGRRT | Blunt (dsDNA) | 20 |
| Cas9 (C. jejuni) | NNNNRYAC | Blunt (dsDNA) | 22 |
| Cas 9 (S. thermophilus) | NNAGAAW | Blunt (dsDNA) | 20 |
| Cas 9 (N. meningitidis) | NNNNGATT | Blunt (dsDNA) | 24 |
| Cas 9 (F. novicida) | NGG | Blunt (dsDNA) | 21 |
| Cas12a (L. bacterium) | TTTV | 5′ staggered | 23-25 |
| | | (dsDNA/ssDNA) | |
| Cas12a (Acidaminococcus | TTTN | 5′ staggered | 24 |
| sp.) | | (dsDNA/ssDNA) | |
| Cas3 (E. coli) | CTT/CCT/CAT/CTC | None/blunt (ssDNA) | 32 |

See generally Wang, J., Zhang, C., & Feng, B. (2020). Journal of Cellular and Molecular Medicine, 24 (6), 3256-3270, where N=any nucleotide; R=any purine (A or G); Y=any pyrimidine (C or T); W=A or T; V=A, C or G.

In one aspect, the CRISPR/Cas system of the present disclosure comprises at least one RNA-guided nuclease (e.g. a Cas protein) derived from one or more of the following selected bacterial genera: Corynebacterium, Sutterella, Legionella, Treponema, Filifactor, Eubacterium, Streptococcus, Lactobacillus, Mycoplasma, Bacteroides, Flavobacterium, Spirochaeta, Azospirillum, Gluconacetobacter, Neisseria, Roseburia, Parvibaculum, Nitratifractor, Campylobacter, Pseudomonas, Streptomyces, Staphylococcus, Francisella, Acidaminococcus, Lachnospiraceae, Leptotrichia, and Prevotella. In some embodiments, the Cas protein is derived from Deltaproteobacteria or Planctomycetes bacterial species.

Some aspects of the present disclosure provide strategies, methods, compositions, and treatment modalities for altering a targeted sequence within a gene locus (e.g., altering the sequence of wild type and/or of a mutant sequences within a cell or within a mammal) by insertion or deletion of one or more nucleotides mediated by an RNA-guided nuclease and one or more guide RNAs (gRNAs), resulting in loss of function of the targeted gene product. In some embodiments, the loss of function results in “knocking out” the gene of interest (i.e., generation of a “knock out”) by ablating gene expression. In some embodiments, the loss function results in a non-functional gene product (i.e., a gene product without all functionality of the wildtype gene product). In some embodiments, the loss of function results in expression of gene product with different characteristics (e.g., different binding affinity or different cellular localization).

In certain embodiments, the targeted gene is selected from TGFB1, TGFBR1, TGFBR2, and combinations thereof. In some embodiments, any region of the targeted gene (e.g., a promoter region, a 5′ untranslated region, a 3′ untranslated region, an exon, an intron, or an exon/intron border) is targeted by an RNA-guided nuclease to alter the gene, its expression or both. In some embodiments, a non-coding region of the targeted gene (e.g., an enhancer region, a promoter region, an intron, 5′ UTR, 3′ UTR, polyadenylation signal) is targeted to alter the gene, its expression or both.

### CRISPR Guide RNAs:

In one aspect, the CRISPR/Cas system of the present disclosure further provides a gRNA molecule (e.g., an isolated or non-naturally occurring RNA molecule) that interacts with the RNA-guided nuclease. In certain embodiments, the gRNA is an sgRNA comprising a spacer sequence comprising a nucleotide sequence which is complementary to a sequence in a target nucleic acid. In some embodiments, the sgRNA further comprises an RNA scaffold (a chimeric fusion of the 3′ end of crRNA and 5′ end of the tracrRNA) that interacts with the RNA-guided nuclease, such that the spacer sequence is positioned to scan a target nucleic acid for complementarity. In some embodiments, the system is further, optionally, comprised of an oligonucleotide-an HDR template with homology to either side of the target position. See Bloh, K., & Rivera-Torres, N, at 3836.

In an embodiment, the RNA-guided nuclease and sgRNA are configured to orient an associated nuclease such that a cleavage event, (e.g., a double-strand break or a single strand break) occurs sufficiently close to a complementary sequence in the targeted nucleic acid, thereby facilitating an alteration in the nucleic acid sequence. In some embodiments, the spacer sequence is 20 nucleotides in length. In some embodiments, the spacer sequence is 14, 15, 16, 17, 18, 19, 20, 21, 22, 23, 24, 25, 26, 27, 28, 29, or 30 nucleotides in length.

In some embodiments, the spacer sequence orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand DNA break may be positioned upstream or downstream of the complementary sequence in the targeted nucleic acid. In some embodiments, the cleavage event occurs within a targeted gene. In some embodiments, the cleavage event occurs upstream of a targeted gene.

In certain embodiments, a second gRNA molecule, comprising a second spacer sequence orients a second RNA-guided nuclease, such that a cleavage event occurs sufficiently close to a complementary sequence in the targeted nucleic acid, thereby facilitating an alteration in the nucleic acid sequence. In some embodiments, the first gRNA and the second gRNA promote a cleavage event within a single targeted gene. In some embodiments, the first gRNA and the second gRNA promote a cleavage event within different targeted genes. In some embodiments, the second spacer sequence is 20 nucleotides in length. In some embodiments, the second spacer sequence is 14, 15, 16, 17, 18, 19, 20, 21, 22, 23, 24, 25, 26, 27, 28, 29, or 30 nucleotides in length.

In some embodiments, the second spacer sequence orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand DNA break may be positioned upstream or downstream of the complementary sequence in the targeted nucleic acid. In some embodiments, the cleavage event occurs within a targeted gene. In some embodiments, the cleavage event occurs upstream of a targeted gene.

In some embodiments, the targeting domains of the first gRNA and the second gRNA are configured such that a cleavage event is positioned, independently for each of the gRNA molecules, within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of the others cleavage event. In some embodiments, the first gRNA and the second gRNA molecules alter the targeted nucleic acid sequences simultaneously. In some embodiments, the first gRNA and the second gRNA molecules alter the targeted nucleic acid sequences sequentially.

In some embodiments, a single-strand break is accompanied by a second single-strand break, positioned by the spacer sequence of a first gRNA and a second gRNA, respectively. For example, the spacer sequence may orient the associated RNA-guided nucleases such that a cleavage event, (e.g., the two single-strand breaks), are positioned within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of one another. In some embodiments, a first spacer and a second spacer sequence are configured to orient associated RNA-guided nucleases such that, for example, two single-strand breaks occurs at the same position, or within 1, 2, 3, 4, 5, 10, 15, 20, 25, 30, 35, 40, 45, 50 nucleotides of one another, on opposing strands of genomic DNA, thereby essentially approximating a double strand break.

In some embodiments, the nucleic acid encoding one or more spacer sequences is selected from any sequence disclosed in Table 2. In some embodiments, the nucleic acid encoding one or more spacer sequences is selected from any sequence disclosed in Table 3. In some embodiments, the nucleic acid encoding one or more spacer sequences is selected from any sequence disclosed in Table 4. In some embodiments a nucleic acid encodes a second sgRNA molecule. In some embodiments, a nucleic acid encodes a third sgRNA molecule. In some embodiments, a nucleic acid encodes a fourth sgRNA molecule. In certain embodiments, one or more sgRNAs is encoded by a nucleic acid selected from any one of SEQ ID NOs: 1-519.

In certain embodiments, a nucleic acid may comprise (a) a sequence encoding a first sgRNA, comprising a spacer sequence that is complementary with a sequence in a targeted gene, (b) a sequence encoding a second sgRNA, comprising a spacer sequence that is complementary with a sequence in a second targeted gene, and (c) a sequence encoding an RNA-guided nuclease (e.g., Cas9). Optionally, (d) and (e) are sequences encoding a third sgRNA and a fourth sgRNA, respectively. In some embodiments, the second targeted gene is the same as the first targeted gene. In other embodiments, the second targeted gene is different from the first targeted gene. In some embodiments, (a), (b), and (c) are encoded within the same nucleic acid molecule (e.g., the same vector). In some embodiments, (a) and (b) are encoded within the same nucleic acid molecule. In some embodiments, (a), (b) and (d) are encoded within the same nucleic acid molecule. In some embodiments, (a), (b) and (e) are encoded within the same nucleic acid molecule. In some embodiments, (a), (b), (d) and (e) are encoded within the same nucleic acid molecule. In some embodiments, (a), (b), and (c) are encoded within separate nucleic acid molecules. When more than two sgRNAs are used, any combination of (a), (b), (c), (d) and (e) may be encoded within a single or separate nucleic acid molecules.

In one aspect, the nucleic acid molecules (i.e., those encoding (a), (b), (c), (d) or (e)) are delivered to a target cell (i.e., any combination of the encoded RNA-guided nuclease of (c) and at least one encoded gRNA molecule of (a), (b), (d), or (e) contact a target cell). In some embodiments, said nucleic acid molecules are delivered to a target cell in vivo. In other embodiments, said nucleic acid molecules are delivered to a target cell ex vivo. In some embodiments, said nucleic acid molecules are delivered to a target cell in vitro. In certain embodiments, said nucleic acid molecules are delivered to a target cell as DNA. In other embodiments, said nucleic acid molecules are delivered to a target cell as RNA (e.g., mRNA). In some embodiments, the products of said nucleic acid molecules are delivered as an assembled ribonucleoprotein (RNP).

In some embodiments, contacting a target cell comprises delivering said RNA-guided nuclease of (c), as a protein with at least one said nucleic acid molecules selected from (a), (b), (d), and (e). In some embodiments, contacting a target cell comprises delivering said encoded RNA-guided nuclease of (c), as DNA with at least one said nucleic acid molecules selected from (a), (b), (d), and (e). In some embodiments, contacting a target cell comprises delivering said encoded RNA-guided nuclease of (c), as mRNA with at least one said nucleic acid molecules selected from (a), (b), (d), and (e).

In certain embodiments, CRISPR components are delivered to a target cell via nanoparticles. Exemplary nanoparticles that may be used with all CRISPR/Cas systems disclosed herein include, at least, lipid nanoparticles or liposomes, hydrogel nanoparticles, metalorganic nanoparticles, gold nanoparticles, and magnetic nanoparticles. See generally Xu, C. F., et al. (2021). Advanced Drug Delivery Reviews, 168, 3-29.

### B. TALEN

In one aspect, the present disclosure contemplates use of methods, components, and compositions relating to Transcription Activator-Like Effector Nucleases (TALENs) to effectuate augmentation of a ‘nucleic acid sequence (e.g., a targeted gene.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, KY, USA), and Life Technologies (Grand Island, NY, USA). TALE and TALEN methods suitable for use in the present disclosure are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

Non-limiting examples of genes that may be silenced or inhibited by permanently gene-editing via a TALE method include TGFB1, TGFBR1, TGFBR2, and combinations thereof. Non-limiting examples of genes that may be augmented such that their resultant products function as decoys or dominant negatives by permanently gene-editing via a TALE method include TGFB1, TGFBR1, TGFBR2, and combinations thereof. Non-limiting examples of genes that may be enhanced by permanently gene-editing via a TALE method include TGFB1, TGFBR1, TGFBR2, and combinations thereof. In an aspect, the disclosure provides compositions for up-regulation of protein receptors (including wildtype or genetically edited), including those that bind to anti-inflammatory cytokines via a TALE method.

Examples of systems, methods, and compositions for altering the expression of a target gene sequence by a TALE method, and which may be used in accordance with embodiments of the present disclosure, are described in U.S. Pat. No. 8,586,526, which is incorporated by reference herein.

### C. Zinc-Finger Nucleases (ZFN)

In one aspect, the present disclosure contemplates use of methods, components, and compositions relating to zinc-finger nucleases (ZFNs) to effectuate augmentation of a nucleic acid sequence (e.g., a targeted gene).

An individual zinc finger contains approximately 30 amino acids in a conserved ββa configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, CA, USA) has developed a propriety platform (CompoZr®) for zinc-finger construction in partnership with Sigma-Aldrich (St. Louis, MO, USA).

Non-limiting examples of genes that may be silenced or inhibited by permanently gene-editing via a zinc finger method include TGFB1, TGFBR1, TGFBR2, and combinations thereof. Non-limiting examples of genes that may be augmented such that their resultant products function as decoys or dominant negatives by permanently gene-editing via a zinc finger method include TGFB1, TGFBR1, TGFBR2, and combinations thereof. Non-limiting examples of genes that may be enhanced by permanently gene-editing via a zinc finger method include TGFB1, TGFBR1, TGFBR2, and combinations thereof. In an aspect, the disclosure provides compositions for up-regulation of protein receptors (including wildtype or genetically edited), including those that bind to anti-inflammatory cytokines via a zinc finger method.

Examples of systems, methods, and compositions for altering the expression of a target gene sequence by a zinc finger method, which may be used in accordance with embodiments of the present disclosure, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

Other examples of systems, methods, and compositions for altering the expression of a target gene sequence by a zinc finger method, which may be used in accordance with embodiments of the present disclosure, are described in Beane, et al., Mol. Therapy, 2015, 23 1380-1390, the disclosure of which is incorporated by reference herein.

### IV. Fibrosis/Scarring

### A. Introduction

Musculoskeletal tissue is particularly prone to fibrosis and/or scarring following trauma. In many instances this trauma may be a surgical procedure. Indeed, formation of musculoskeletal scar tissue presents several issues, including limiting range of motion, causing pain and complicating subsequent surgical access to the site of injury. Without wishing to be bound to a particular theory, increased expression of TGFB1, among other factors is observed in fascial fibroblasts at the site of such musculoskeletal trauma. See, Ihn, H. (2019). Allergology International, 68 (4), 437-439. This expression pattern correlated with higher expression of type I collagen and fibronectin-contributors to fibrotic processes.

In one aspect, the compositions and methods herein described are directed to treatment of excess fibrosis and/or scarring. In some embodiments, this scarring is the result of disease (e.g. liver fibrosis) or trauma (e.g. tendon damage). In some embodiments, the fibrosis and/or scarring is the result of a surgical procedure. In some embodiments, the surgical procedure is ligament reconstruction. In some embodiments, the surgical procedure is anterior cruciate ligament (ACL) reconstruction. In some embodiments, the surgical procedure is autograft ACL reconstruction. In some embodiments, the surgical procedure is allograft ACL reconstruction. In some embodiments, the surgical procedure is repair of a fracture. In some embodiments, the surgical procedure is total knee arthroplasty (TKA). In some embodiments, the surgical procedure is microdiscectomy.

In some embodiments, the fibrosis and/or scarring is the result of a condition, which may be induced or exacerbated by a surgical procedure. In some embodiments, the condition contributing to musculoskeletal fibrosis and/or scarring is knee arthrofibrosis. In some embodiments, the condition contributing to musculoskeletal fibrosis and/or scarring is intra-articular fibrous nodules. In some embodiments, the condition contributing to musculoskeletal fibrosis and/or is epidural fibrosis.

### B. Fibrosis

In one aspect, the present disclosure encompasses treatments for fibrosis. In some embodiments, the CRISPR gene-editing system for the treatment of fibrosis comprises one or more sgRNAs targeting TGFB1. In some embodiments, the CRISPR gene-editing system for the treatment of fibrosis comprises one or more sgRNAs targeting hTGFB1. In some embodiments, the CRISPR gene-editing system for the treatment of fibrosis comprises one or more sgRNAs targeting cTGFB1. In some embodiments, the CRISPR gene-editing system for the treatment of fibrosis comprises one or more sgRNAs targeting eTGFB1. In some embodiments, the CRISPR gene-editing system for the treatment of fibrosis comprises one or more sgRNAs targeting fTGFB1.

In some embodiments, the CRISPR gene-editing system for the treatment of fibrosis comprises one or more sgRNAs targeting exon 1 of hTGFB1. In some embodiments, the CRISPR gene-editing system for the treatment of fibrosis comprises one or more sgRNAs targeting exon 2 of hTGFB1. In some embodiments, the CRISPR gene-editing system for the treatment of fibrosis comprises one or more sgRNAs targeting exon 3 of hTGFB1. In some embodiments, the CRISPR gene-editing system for the treatment of fibrosis comprises one or more sgRNAs targeting exon 4 of hTGFB1. In some embodiments, the CRISPR gene-editing system for the treatment of fibrosis comprises one or more sgRNAs targeting exon 5 of hTGFB1. In some embodiments, the CRISPR gene-editing system for the treatment of fibrosis comprises one or more sgRNAs targeting exon 6 of hTGFB1. In some embodiments, the CRISPR gene-editing system for the treatment of fibrosis comprises one or more sgRNAs targeting exon 7 of hTGFB1.

In some embodiments, the CRISPR gene-editing system for the treatment of fibrosis comprises one or more sgRNAs targeting exon 1 of cTGFB1. In some embodiments, the CRISPR gene-editing system for the treatment of fibrosis comprises one or more sgRNAs targeting exon 2 of cTGFB1. In some embodiments, the CRISPR gene-editing system for the treatment of fibrosis comprises one or more sgRNAs targeting exon 3 of cTGFB1. In some embodiments, the CRISPR gene-editing system for the treatment of fibrosis comprises one or more sgRNAs targeting exon 4 of cTGFB1. In some embodiments, the CRISPR gene-editing system for the treatment of fibrosis comprises one or more sgRNAs targeting exon 5 of cTGFB1. In some embodiments, the CRISPR gene-editing system for the treatment of fibrosis comprises one or more sgRNAs targeting exon 6 of cTGFB1. In some embodiments, the CRISPR gene-editing system for the treatment of fibrosis comprises one or more sgRNAs targeting exon 7 of cTGFB1.

In some embodiments, the CRISPR gene-editing system for the treatment of fibrosis comprises one or more sgRNAs targeting exon 1 of eTGFB1. In some embodiments, the CRISPR gene-editing system for the treatment of fibrosis comprises one or more sgRNAs targeting exon 2 of eTGFB1. In some embodiments, the CRISPR gene-editing system for the treatment of fibrosis comprises one or more sgRNAs targeting exon 3 of eTGFB1. In some embodiments, the CRISPR gene-editing system for the treatment of fibrosis comprises one or more sgRNAs targeting exon 4 of eTGFB1. In some embodiments, the CRISPR gene-editing system for the treatment of fibrosis comprises one or more sgRNAs targeting exon 5 of eTGFB1. In some embodiments, the CRISPR gene-editing system for the treatment of fibrosis comprises one or more sgRNAs targeting exon 6 of eTGFB1. In some embodiments, the CRISPR gene-editing system for the treatment of fibrosis comprises one or more sgRNAs targeting exon 7 of eTGFB1.

In some embodiments, the CRISPR gene-editing system for the treatment of fibrosis comprises one or more sgRNAs targeting exon 1 of fTGFB1. In some embodiments, the CRISPR gene-editing system for the treatment of fibrosis comprises one or more sgRNAs targeting exon 2 of fTGFB1. In some embodiments, the CRISPR gene-editing system for the treatment of fibrosis comprises one or more sgRNAs targeting exon 3 of fTGFB1. In some embodiments, the CRISPR gene-editing system for the treatment of fibrosis comprises one or more sgRNAs targeting exon 4 of fTGFB1. In some embodiments, the CRISPR gene-editing system for the treatment of fibrosis comprises one or more sgRNAs targeting exon 5 of fTGFB1. In some embodiments, the CRISPR gene-editing system for the treatment of fibrosis comprises one or more sgRNAs targeting exon 6 of fTGFB1. In some embodiments, the CRISPR gene-editing system for the treatment of fibrosis comprises one or more sgRNAs targeting exon 7 of fTGFB1.

In some embodiments, the CRISPR gene-editing system for the treatment of fibrosis comprises one or more sgRNAs targeting TGFBR1. In some embodiments, the CRISPR gene-editing system for the treatment of fibrosis comprises one or more sgRNAs targeting hTGFBR1. In some embodiments, the CRISPR gene-editing system for the treatment of fibrosis comprises one or more sgRNAs targeting cTGFBR1. In some embodiments, the CRISPR gene-editing system for the treatment of fibrosis comprises one or more sgRNAs targeting e TGFBR1. In some embodiments, the CRISPR gene-editing system for the treatment of fibrosis comprises one or more sgRNAs targeting fTGFBR1.

In some embodiments, the CRISPR gene-editing system for the treatment of fibrosis comprises one or more sgRNAs targeting exon 1 of hTGFBR1. In some embodiments, the CRISPR gene-editing system for the treatment of fibrosis comprises one or more sgRNAs targeting exon 2 of hTGFBR1. In some embodiments, the CRISPR gene-editing system for the treatment of fibrosis comprises one or more sgRNAs targeting exon 3 of hTGFBR1. In some embodiments, the CRISPR gene-editing system for the treatment of fibrosis comprises one or more sgRNAs targeting exon 4 of hTGFBR1. In some embodiments, the CRISPR gene-editing system for the treatment of fibrosis comprises one or more sgRNAs targeting exon 5 of hTGFBR1. In some embodiments, the CRISPR gene-editing system for the treatment of fibrosis comprises one or more sgRNAs targeting exon 6 of hTGFBR1. In some embodiments, the CRISPR gene-editing system for the treatment of fibrosis comprises one or more sgRNAs targeting exon 7 of hTGFBR1. In some embodiments, the CRISPR gene-editing system for the treatment of fibrosis comprises one or more sgRNAs targeting exon 8 of hTGFBR1. In some embodiments, the CRISPR gene-editing system for the treatment of fibrosis comprises one or more sgRNAs targeting exon 9 of hTGFBR1. In some embodiments, the CRISPR gene-editing system for the treatment of fibrosis comprises one or more sgRNAs targeting exon 10 of hTGFBR1. In some embodiments, the CRISPR gene-editing system for the treatment of fibrosis comprises one or more sgRNAs targeting exon 11 of hTGFBR1.

In some embodiments, the CRISPR gene-editing system for the treatment of fibrosis comprises one or more sgRNAs targeting exon 1 of cTGFBR1. In some embodiments, the CRISPR gene-editing system for the treatment of fibrosis comprises one or more sgRNAs targeting exon 2 of cTGFBR1. In some embodiments, the CRISPR gene-editing system for the treatment of fibrosis comprises one or more sgRNAs targeting exon 3 of cTGFBR1. In some embodiments, the CRISPR gene-editing system for the treatment of fibrosis comprises one or more sgRNAs targeting exon 4 of cTGFBR1. In some embodiments, the CRISPR gene-editing system for the treatment of fibrosis comprises one or more sgRNAs targeting exon 5 of cTGFBR1. In some embodiments, the CRISPR gene-editing system for the treatment of fibrosis comprises one or more sgRNAs targeting exon 6 of cTGFBR1. In some embodiments, the CRISPR gene-editing system for the treatment of fibrosis comprises one or more sgRNAs targeting exon 7 of cTGFBR1. In some embodiments, the CRISPR gene-editing system for the treatment of fibrosis comprises one or more sgRNAs targeting exon 8 of cTGFBR1. In some embodiments, the CRISPR gene-editing system for the treatment of fibrosis comprises one or more sgRNAs targeting exon 9 of cTGFBR1.

In some embodiments, the CRISPR gene-editing system for the treatment of fibrosis comprises one or more sgRNAs targeting exon 1 of eTGFBR1. In some embodiments, the CRISPR gene-editing system for the treatment of fibrosis comprises one or more sgRNAs targeting exon 2 of eTGFBR1. In some embodiments, the CRISPR gene-editing system for the treatment of fibrosis comprises one or more sgRNAs targeting exon 3 of eTGFBR1. In some embodiments, the CRISPR gene-editing system for the treatment of fibrosis comprises one or more sgRNAs targeting exon 4 of eTGFBR1. In some embodiments, the CRISPR gene-editing system for the treatment of fibrosis comprises one or more sgRNAs targeting exon 5 of eTGFBR1. In some embodiments, the CRISPR gene-editing system for the treatment of fibrosis comprises one or more sgRNAs targeting exon 6 of eTGFBR1. In some embodiments, the CRISPR gene-editing system for the treatment of fibrosis comprises one or more sgRNAs targeting exon 7 of eTGFBR1. In some embodiments, the CRISPR gene-editing system for the treatment of fibrosis comprises one or more sgRNAs targeting exon 8 of eTGFBR1. In some embodiments, the CRISPR gene-editing system for the treatment of fibrosis comprises one or more sgRNAs targeting exon 9 of eTGFBR1. In some embodiments, the CRISPR gene-editing system for the treatment of fibrosis comprises one or more sgRNAs targeting exon 10 of eTGFBR1. In some embodiments, the CRISPR gene-editing system for the treatment of fibrosis comprises one or more sgRNAs targeting exon 11 of eTGFBR1. In some embodiments, the CRISPR gene-editing system for the treatment of fibrosis comprises one or more sgRNAs targeting exon 12 of eTGFBR1.

In some embodiments, the CRISPR gene-editing system for the treatment of fibrosis comprises one or more sgRNAs targeting exon 1 of fTGFBR1. In some embodiments, the CRISPR gene-editing system for the treatment of fibrosis comprises one or more sgRNAs targeting exon 2 of fTGFBR1. In some embodiments, the CRISPR gene-editing system for the treatment of fibrosis comprises one or more sgRNAs targeting exon 3 of fTGFBR1. In some embodiments, the CRISPR gene-editing system for the treatment of fibrosis comprises one or more sgRNAs targeting exon 4 of fTGFBR1. In some embodiments, the CRISPR gene-editing system for the treatment of fibrosis comprises one or more sgRNAs targeting exon 5 of fTGFBR1. In some embodiments, the CRISPR gene-editing system for the treatment of fibrosis comprises one or more sgRNAs targeting exon 6 of fTGFBR1. In some embodiments, the CRISPR gene-editing system for the treatment of fibrosis comprises one or more sgRNAs targeting exon 7 of fTGFBR1. In some embodiments, the CRISPR gene-editing system for the treatment of fibrosis comprises one or more sgRNAs targeting exon 8 of fTGFBR1. In some embodiments, the CRISPR gene-editing system for the treatment of fibrosis comprises one or more sgRNAs targeting exon 9 of fTGFBR1. In some embodiments, the CRISPR gene-editing system for the treatment of fibrosis comprises one or more sgRNAs targeting exon 10 of fTGFBR1. In some embodiments, the CRISPR gene-editing system for the treatment of fibrosis comprises one or more sgRNAs targeting exon 11 of fTGFBR1.

In some embodiments, the CRISPR gene-editing system for the treatment of fibrosis comprises one or more sgRNAs targeting TGFBR2. In some embodiments, the CRISPR gene-editing system for the treatment of fibrosis comprises one or more sgRNAs targeting hTGFBR2. In some embodiments, the CRISPR gene-editing system for the treatment of fibrosis comprises one or more sgRNAs targeting cTGFBR2. In some embodiments, the CRISPR gene-editing system for the treatment of fibrosis comprises one or more sgRNAs targeting eTGFBR2. In some embodiments, the CRISPR gene-editing system for the treatment of fibrosis comprises one or more sgRNAs targeting fTGFBR2.

In some embodiments, the CRISPR gene-editing system for the treatment of fibrosis comprises one or more sgRNAs targeting exon 1 of hTGFBR2. In some embodiments, the CRISPR gene-editing system for the treatment of fibrosis comprises one or more sgRNAs targeting exon 2 of hTGFBR2. In some embodiments, the CRISPR gene-editing system for the treatment of fibrosis comprises one or more sgRNAs targeting exon 3 of hTGFBR2. In some embodiments, the CRISPR gene-editing system for the treatment of fibrosis comprises one or more sgRNAs targeting exon 4 of hTGFBR2. In some embodiments, the CRISPR gene-editing system for the treatment of fibrosis comprises one or more sgRNAs targeting exon 5 of hTGFBR2. In some embodiments, the CRISPR gene-editing system for the treatment of fibrosis comprises one or more sgRNAs targeting exon 6 of hTGFBR2. In some embodiments, the CRISPR gene-editing system for the treatment of fibrosis comprises one or more sgRNAs targeting exon 7 of hTGFBR2. In some embodiments, the CRISPR gene-editing system for the treatment of fibrosis comprises one or more sgRNAs targeting exon 8 of hTGFBR2. In some embodiments, the CRISPR gene-editing system for the treatment of fibrosis comprises one or more sgRNAs targeting exon 9 of hTGFBR2.

In some embodiments, the CRISPR gene-editing system for the treatment of fibrosis comprises one or more sgRNAs targeting exon 1 of cTGFBR2. In some embodiments, the CRISPR gene-editing system for the treatment of fibrosis comprises one or more sgRNAs targeting exon 2 of cTGFBR2. In some embodiments, the CRISPR gene-editing system for the treatment of fibrosis comprises one or more sgRNAs targeting exon 3 of cTGFBR2. In some embodiments, the CRISPR gene-editing system for the treatment of fibrosis comprises one or more sgRNAs targeting exon 4 of cTGFBR2. In some embodiments, the CRISPR gene-editing system for the treatment of fibrosis comprises one or more sgRNAs targeting exon 5 of cTGFBR2. In some embodiments, the CRISPR gene-editing system for the treatment of fibrosis comprises one or more sgRNAs targeting exon 6 of cTGFBR2. In some embodiments, the CRISPR gene-editing system for the treatment of fibrosis comprises one or more sgRNAs targeting exon 7 of cTGFBR2. In some embodiments, the CRISPR gene-editing system for the treatment of fibrosis comprises one or more sgRNAs targeting exon 8 of cTGFBR2.

In some embodiments, the CRISPR gene-editing system for the treatment of fibrosis comprises one or more sgRNAs targeting exon 1 of eTGFBR2. In some embodiments, the CRISPR gene-editing system for the treatment of fibrosis comprises one or more sgRNAs targeting exon 2 of eTGFBR2. In some embodiments, the CRISPR gene-editing system for the treatment of fibrosis comprises one or more sgRNAs targeting exon 3 of eTGFBR2. In some embodiments, the CRISPR gene-editing system for the treatment of fibrosis comprises one or more sgRNAs targeting exon 4 of eTGFBR2. In some embodiments, the CRISPR gene-editing system for the treatment of fibrosis comprises one or more sgRNAs targeting exon 5 of eTGFBR2. In some embodiments, the CRISPR gene-editing system for the treatment of fibrosis comprises one or more sgRNAs targeting exon 6 of eTGFBR2. In some embodiments, the CRISPR gene-editing system for the treatment of fibrosis comprises one or more sgRNAs targeting exon 7 of eTGFBR2. In some embodiments, the CRISPR gene-editing system for the treatment of fibrosis comprises one or more sgRNAs targeting exon 8 of eTGFBR2. In some embodiments, the CRISPR gene-editing system for the treatment of fibrosis comprises one or more sgRNAs targeting exon 9 of eTGFBR2. In some embodiments, the CRISPR gene-editing system for the treatment of fibrosis comprises one or more sgRNAs targeting exon 10 of eTGFBR2.

In some embodiments, the CRISPR gene-editing system for the treatment of fibrosis comprises one or more sgRNAs targeting exon 1 of fTGFBR2. In some embodiments, the CRISPR gene-editing system for the treatment of fibrosis comprises one or more sgRNAs targeting exon 2 of fTGFBR2. In some embodiments, the CRISPR gene-editing system for the treatment of fibrosis comprises one or more sgRNAs targeting exon 3 of fTGFBR2. In some embodiments, the CRISPR gene-editing system for the treatment of fibrosis comprises one or more sgRNAs targeting exon 4 of fTGFBR2. In some embodiments, the CRISPR gene-editing system for the treatment of fibrosis comprises one or more sgRNAs targeting exon 5 of fTGFBR2. In some embodiments, the CRISPR gene-editing system for the treatment of fibrosis comprises one or more sgRNAs targeting exon 6 of fTGFBR2. In some embodiments, the CRISPR gene-editing system for the treatment of fibrosis comprises one or more sgRNAs targeting exon 7 of fTGFBR2. In some embodiments, the CRISPR gene-editing system for the treatment of fibrosis comprises one or more sgRNAs targeting exon 8 of fTGFBR2.

### C. Musculoskeletal Scarring

In one aspect, the present disclosure encompasses treatments for musculoskeletal scarring. In some embodiments, the CRISPR gene-editing system for the treatment of musculoskeletal scarring comprises one or more sgRNAs targeting TGFB1. In some embodiments, the CRISPR gene-editing system for the treatment of musculoskeletal scarring comprises one or more sgRNAs targeting hTGFB1. In some embodiments, the CRISPR gene-editing system for the treatment of musculoskeletal scarring comprises one or more sgRNAs targeting cTGFB1. In some embodiments, the CRISPR gene-editing system for the treatment of musculoskeletal scarring comprises one or more sgRNAs targeting e TGFB1. In some embodiments, the CRISPR gene-editing system for the treatment of musculoskeletal scarring comprises one or more sgRNAs targeting fTGFB1.

In some embodiments, the CRISPR gene-editing system for the treatment of musculoskeletal scarring comprises one or more sgRNAs targeting exon 1 of hTGFB1. In some embodiments, the CRISPR gene-editing system for the treatment of musculoskeletal scarring comprises one or more sgRNAs targeting exon 2 of hTGFB1. In some embodiments, the CRISPR gene-editing system for the treatment of musculoskeletal scarring comprises one or more sgRNAs targeting exon 3 of hTGFB1. In some embodiments, the CRISPR gene-editing system for the treatment of musculoskeletal scarring comprises one or more sgRNAs targeting exon 4 of hTGFB1. In some embodiments, the CRISPR gene-editing system for the treatment of musculoskeletal scarring comprises one or more sgRNAs targeting exon 5 of hTGFB1. In some embodiments, the CRISPR gene-editing system for the treatment of musculoskeletal scarring comprises one or more sgRNAs targeting exon 6 of hTGFB1. In some embodiments, the CRISPR gene-editing system for the treatment of musculoskeletal scarring comprises one or more sgRNAs targeting exon 7 of hTGFB1.

In some embodiments, the CRISPR gene-editing system for the treatment of musculoskeletal scarring comprises one or more sgRNAs targeting exon 1 of cTGFB1. In some embodiments, the CRISPR gene-editing system for the treatment of musculoskeletal scarring comprises one or more sgRNAs targeting exon 2 of cTGFB1. In some embodiments, the CRISPR gene-editing system for the treatment of musculoskeletal scarring comprises one or more sgRNAs targeting exon 3 of cTGFB1. In some embodiments, the CRISPR gene-editing system for the treatment of musculoskeletal scarring comprises one or more sgRNAs targeting exon 4 of cTGFB1. In some embodiments, the CRISPR gene-editing system for the treatment of musculoskeletal scarring comprises one or more sgRNAs targeting exon 5 of cTGFB1. In some embodiments, the CRISPR gene-editing system for the treatment of musculoskeletal scarring comprises one or more sgRNAs targeting exon 6 of cTGFB1. In some embodiments, the CRISPR gene-editing system for the treatment of musculoskeletal scarring comprises one or more sgRNAs targeting exon 7 of cTGFB1.

In some embodiments, the CRISPR gene-editing system for the treatment of musculoskeletal scarring comprises one or more sgRNAs targeting exon 1 of eTGFB1. In some embodiments, the CRISPR gene-editing system for the treatment of musculoskeletal scarring comprises one or more sgRNAs targeting exon 2 of eTGFB1. In some embodiments, the CRISPR gene-editing system for the treatment of musculoskeletal scarring comprises one or more sgRNAs targeting exon 3 of eTGFB1. In some embodiments, the CRISPR gene-editing system for the treatment of musculoskeletal scarring comprises one or more sgRNAs targeting exon 4 of eTGFB1. In some embodiments, the CRISPR gene-editing system for the treatment of musculoskeletal scarring comprises one or more sgRNAs targeting exon 5 of eTGFB1. In some embodiments, the CRISPR gene-editing system for the treatment of musculoskeletal scarring comprises one or more sgRNAs targeting exon 6 of eTGFB1. In some embodiments, the CRISPR gene-editing system for the treatment of musculoskeletal scarring comprises one or more sgRNAs targeting exon 7 of eTGFB1.

In some embodiments, the CRISPR gene-editing system for the treatment of musculoskeletal scarring comprises one or more sgRNAs targeting exon 1 of fTGFB1. In some embodiments, the CRISPR gene-editing system for the treatment of musculoskeletal scarring comprises one or more sgRNAs targeting exon 2 of fTGFB1. In some embodiments, the CRISPR gene-editing system for the treatment of musculoskeletal scarring comprises one or more sgRNAs targeting exon 3 of fTGFB1. In some embodiments, the CRISPR gene-editing system for the treatment of musculoskeletal scarring comprises one or more sgRNAs targeting exon 4 of fTGFB1. In some embodiments, the CRISPR gene-editing system for the treatment of musculoskeletal scarring comprises one or more sgRNAs targeting exon 5 of fTGFB1. In some embodiments, the CRISPR gene-editing system for the treatment of musculoskeletal scarring comprises one or more sgRNAs targeting exon 6 of fTGFB1. In some embodiments, the CRISPR gene-editing system for the treatment of musculoskeletal scarring comprises one or more sgRNAs targeting exon 7 of fTGFB1.

In some embodiments, the CRISPR gene-editing system for the treatment of musculoskeletal scarring comprises one or more sgRNAs targeting TGFBR1. In some embodiments, the CRISPR gene-editing system for the treatment of musculoskeletal scarring comprises one or more sgRNAs targeting hTGFBR1. In some embodiments, the CRISPR gene-editing system for the treatment of musculoskeletal scarring comprises one or more sgRNAs targeting cTGFBR1. In some embodiments, the CRISPR gene-editing system for the treatment of musculoskeletal scarring comprises one or more sgRNAs targeting e TGFBR1. In some embodiments, the CRISPR gene-editing system for the treatment of musculoskeletal scarring comprises one or more sgRNAs targeting fTGFBR1.

In some embodiments, the CRISPR gene-editing system for the treatment of musculoskeletal scarring comprises one or more sgRNAs targeting exon 1 of hTGFBR1. In some embodiments, the CRISPR gene-editing system for the treatment of musculoskeletal scarring comprises one or more sgRNAs targeting exon 2 of hTGFBR1. In some embodiments, the CRISPR gene-editing system for the treatment of musculoskeletal scarring comprises one or more sgRNAs targeting exon 3 of hTGFBR1. In some embodiments, the CRISPR gene-editing system for the treatment of musculoskeletal scarring comprises one or more sgRNAs targeting exon 4 of hTGFBR1. In some embodiments, the CRISPR gene-editing system for the treatment of musculoskeletal scarring comprises one or more sgRNAs targeting exon 5 of hTGFBR1. In some embodiments, the CRISPR gene-editing system for the treatment of musculoskeletal scarring comprises one or more sgRNAs targeting exon 6 of hTGFBR1. In some embodiments, the CRISPR gene-editing system for the treatment of musculoskeletal scarring comprises one or more sgRNAs targeting exon 7 of hTGFBR1. In some embodiments, the CRISPR gene-editing system for the treatment of musculoskeletal scarring comprises one or more sgRNAs targeting exon 8 of hTGFBR1. In some embodiments, the CRISPR gene-editing system for the treatment of musculoskeletal scarring comprises one or more sgRNAs targeting exon 9 of hTGFBR1. In some embodiments, the CRISPR gene-editing system for the treatment of musculoskeletal scarring comprises one or more sgRNAs targeting exon 10 of hTGFBR1. In some embodiments, the CRISPR gene-editing system for the treatment of musculoskeletal scarring comprises one or more sgRNAs targeting exon 11 of hTGFBR1.

In some embodiments, the CRISPR gene-editing system for the treatment of musculoskeletal scarring comprises one or more sgRNAs targeting exon 1 of cTGFBR1. In some embodiments, the CRISPR gene-editing system for the treatment of musculoskeletal scarring comprises one or more sgRNAs targeting exon 2 of cTGFBR1. In some embodiments, the CRISPR gene-editing system for the treatment of musculoskeletal scarring comprises one or more sgRNAs targeting exon 3 of cTGFBR1. In some embodiments, the CRISPR gene-editing system for the treatment of musculoskeletal scarring comprises one or more sgRNAs targeting exon 4 of cTGFBR1. In some embodiments, the CRISPR gene-editing system for the treatment of musculoskeletal scarring comprises one or more sgRNAs targeting exon 5 of cTGFBR1. In some embodiments, the CRISPR gene-editing system for the treatment of musculoskeletal scarring comprises one or more sgRNAs targeting exon 6 of cTGFBR1. In some embodiments, the CRISPR gene-editing system for the treatment of musculoskeletal scarring comprises one or more sgRNAs targeting exon 7 of cTGFBR1. In some embodiments, the CRISPR gene-editing system for the treatment of musculoskeletal scarring comprises one or more sgRNAs targeting exon 8 of cTGFBR1. In some embodiments, the CRISPR gene-editing system for the treatment of musculoskeletal scarring comprises one or more sgRNAs targeting exon 9 of cTGFBR1.

In some embodiments, the CRISPR gene-editing system for the treatment of musculoskeletal scarring comprises one or more sgRNAs targeting exon 1 of eTGFBR1. In some embodiments, the CRISPR gene-editing system for the treatment of musculoskeletal scarring comprises one or more sgRNAs targeting exon 2 of eTGFBR1. In some embodiments, the CRISPR gene-editing system for the treatment of musculoskeletal scarring comprises one or more sgRNAs targeting exon 3 of eTGFBR1. In some embodiments, the CRISPR gene-editing system for the treatment of musculoskeletal scarring comprises one or more sgRNAs targeting exon 4 of eTGFBR1. In some embodiments, the CRISPR gene-editing system for the treatment of musculoskeletal scarring comprises one or more sgRNAs targeting exon 5 of eTGFBR1. In some embodiments, the CRISPR gene-editing system for the treatment of musculoskeletal scarring comprises one or more sgRNAs targeting exon 6 of eTGFBR1. In some embodiments, the CRISPR gene-editing system for the treatment of musculoskeletal scarring comprises one or more sgRNAs targeting exon 7 of eTGFBR1. In some embodiments, the CRISPR gene-editing system for the treatment of musculoskeletal scarring comprises one or more sgRNAs targeting exon 8 of eTGFBR1. In some embodiments, the CRISPR gene-editing system for the treatment of musculoskeletal scarring comprises one or more sgRNAs targeting exon 9 of eTGFBR1. In some embodiments, the CRISPR gene-editing system for the treatment of musculoskeletal scarring comprises one or more sgRNAs targeting exon 10 of eTGFBR1. In some embodiments, the CRISPR gene-editing system for the treatment of musculoskeletal scarring comprises one or more sgRNAs targeting exon 11 of eTGFBR1. In some embodiments, the CRISPR gene-editing system for the treatment of musculoskeletal scarring comprises one or more sgRNAs targeting exon 12 of eTGFBR1.

In some embodiments, the CRISPR gene-editing system for the treatment of musculoskeletal scarring comprises one or more sgRNAs targeting exon 1 of fTGFBR1. In some embodiments, the CRISPR gene-editing system for the treatment of musculoskeletal scarring comprises one or more sgRNAs targeting exon 2 of fTGFBR1. In some embodiments, the CRISPR gene-editing system for the treatment of musculoskeletal scarring comprises one or more sgRNAs targeting exon 3 of fTGFBR1. In some embodiments, the CRISPR gene-editing system for the treatment of musculoskeletal scarring comprises one or more sgRNAs targeting exon 4 of fTGFBR1. In some embodiments, the CRISPR gene-editing system for the treatment of musculoskeletal scarring comprises one or more sgRNAs targeting exon 5 of fTGFBR1. In some embodiments, the CRISPR gene-editing system for the treatment of musculoskeletal scarring comprises one or more sgRNAs targeting exon 6 of fTGFBR1. In some embodiments, the CRISPR gene-editing system for the treatment of musculoskeletal scarring comprises one or more sgRNAs targeting exon 7 of fTGFBR1. In some embodiments, the CRISPR gene-editing system for the treatment of musculoskeletal scarring comprises one or more sgRNAs targeting exon 8 of fTGFBR1. In some embodiments, the CRISPR gene-editing system for the treatment of musculoskeletal scarring comprises one or more sgRNAs targeting exon 9 of fTGFBR1. In some embodiments, the CRISPR gene-editing system for the treatment of musculoskeletal scarring comprises one or more sgRNAs targeting exon 10 of fTGFBR1. In some embodiments, the CRISPR gene-editing system for the treatment of musculoskeletal scarring comprises one or more sgRNAs targeting exon 11 of fTGFBR1.

In some embodiments, the CRISPR gene-editing system for the treatment of musculoskeletal scarring comprises one or more sgRNAs targeting TGFBR2. In some embodiments, the CRISPR gene-editing system for the treatment of musculoskeletal scarring comprises one or more sgRNAs targeting hTGFBR2. In some embodiments, the CRISPR gene-editing system for the treatment of musculoskeletal scarring comprises one or more sgRNAs targeting cTGFBR2. In some embodiments, the CRISPR gene-editing system for the treatment of musculoskeletal scarring comprises one or more sgRNAs targeting eTGFBR2. In some embodiments, the CRISPR gene-editing system for the treatment of musculoskeletal scarring comprises one or more sgRNAs targeting fTGFBR2.

In some embodiments, the CRISPR gene-editing system for the treatment of musculoskeletal scarring comprises one or more sgRNAs targeting exon 1 of hTGFBR2. In some embodiments, the CRISPR gene-editing system for the treatment of musculoskeletal scarring comprises one or more sgRNAs targeting exon 2 of hTGFBR2. In some embodiments, the CRISPR gene-editing system for the treatment of musculoskeletal scarring comprises one or more sgRNAs targeting exon 3 of hTGFBR2. In some embodiments, the CRISPR gene-editing system for the treatment of musculoskeletal scarring comprises one or more sgRNAs targeting exon 4 of hTGFBR2. In some embodiments, the CRISPR gene-editing system for the treatment of musculoskeletal scarring comprises one or more sgRNAs targeting exon 5 of hTGFBR2. In some embodiments, the CRISPR gene-editing system for the treatment of musculoskeletal scarring comprises one or more sgRNAs targeting exon 6 of hTGFBR2. In some embodiments, the CRISPR gene-editing system for the treatment of musculoskeletal scarring comprises one or more sgRNAs targeting exon 7 of hTGFBR2. In some embodiments, the CRISPR gene-editing system for the treatment of musculoskeletal scarring comprises one or more sgRNAs targeting exon 8 of hTGFBR2. In some embodiments, the CRISPR gene-editing system for the treatment of musculoskeletal scarring comprises one or more sgRNAs targeting exon 9 of hTGFBR2.

In some embodiments, the CRISPR gene-editing system for the treatment of musculoskeletal scarring comprises one or more sgRNAs targeting exon 1 of cTGFBR2. In some embodiments, the CRISPR gene-editing system for the treatment of musculoskeletal scarring comprises one or more sgRNAs targeting exon 2 of cTGFBR2. In some embodiments, the CRISPR gene-editing system for the treatment of musculoskeletal scarring comprises one or more sgRNAs targeting exon 3 of cTGFBR2. In some embodiments, the CRISPR gene-editing system for the treatment of musculoskeletal scarring comprises one or more sgRNAs targeting exon 4 of cTGFBR2. In some embodiments, the CRISPR gene-editing system for the treatment of musculoskeletal scarring comprises one or more sgRNAs targeting exon 5 of cTGFBR2. In some embodiments, the CRISPR gene-editing system for the treatment of musculoskeletal scarring comprises one or more sgRNAs targeting exon 6 of cTGFBR2. In some embodiments, the CRISPR gene-editing system for the treatment of musculoskeletal scarring comprises one or more sgRNAs targeting exon 7 of cTGFBR2. In some embodiments, the CRISPR gene-editing system for the treatment of musculoskeletal scarring comprises one or more sgRNAs targeting exon 8 of cTGFBR2.

In some embodiments, the CRISPR gene-editing system for the treatment of musculoskeletal scarring comprises one or more sgRNAs targeting exon 1 of eTGFBR2. In some embodiments, the CRISPR gene-editing system for the treatment of musculoskeletal scarring comprises one or more sgRNAs targeting exon 2 of eTGFBR2. In some embodiments, the CRISPR gene-editing system for the treatment of musculoskeletal scarring comprises one or more sgRNAs targeting exon 3 of eTGFBR2. In some embodiments, the CRISPR gene-editing system for the treatment of musculoskeletal scarring comprises one or more sgRNAs targeting exon 4 of eTGFBR2. In some embodiments, the CRISPR gene-editing system for the treatment of musculoskeletal scarring comprises one or more sgRNAs targeting exon 5 of eTGFBR2. In some embodiments, the CRISPR gene-editing system for the treatment of musculoskeletal scarring comprises one or more sgRNAs targeting exon 6 of eTGFBR2. In some embodiments, the CRISPR gene-editing system for the treatment of musculoskeletal scarring comprises one or more sgRNAs targeting exon 7 of eTGFBR2. In some embodiments, the CRISPR gene-editing system for the treatment of musculoskeletal scarring comprises one or more sgRNAs targeting exon 8 of eTGFBR2. In some embodiments, the CRISPR gene-editing system for the treatment of musculoskeletal scarring comprises one or more sgRNAs targeting exon 9 of eTGFBR2. In some embodiments, the CRISPR gene-editing system for the treatment of musculoskeletal scarring comprises one or more sgRNAs targeting exon 10 of eTGFBR2.

In some embodiments, the CRISPR gene-editing system for the treatment of musculoskeletal scarring comprises one or more sgRNAs targeting exon 1 of fTGFBR2. In some embodiments, the CRISPR gene-editing system for the treatment of musculoskeletal scarring comprises one or more sgRNAs targeting exon 2 of fTGFBR2. In some embodiments, the CRISPR gene-editing system for the treatment of musculoskeletal scarring comprises one or more sgRNAs targeting exon 3 of fTGFBR2. In some embodiments, the CRISPR gene-editing system for the treatment of musculoskeletal scarring comprises one or more sgRNAs targeting exon 4 of fTGFBR2. In some embodiments, the CRISPR gene-editing system for the treatment of musculoskeletal scarring comprises one or more sgRNAs targeting exon 5 of fTGFBR2. In some embodiments, the CRISPR gene-editing system for the treatment of musculoskeletal scarring comprises one or more sgRNAs targeting exon 6 of fTGFBR2. In some embodiments, the CRISPR gene-editing system for the treatment of musculoskeletal scarring comprises one or more sgRNAs targeting exon 7 of fTGFBR2. In some embodiments, the CRISPR gene-editing system for the treatment of musculoskeletal scarring comprises one or more sgRNAs targeting exon 8 of fTGFBR2.

### V. Delivery

### A. Viral Vectors

In one aspect, the present disclosure encompasses methods of delivery of a CRISPR gene-editing system targeting a gene selected from TGFB1, TGFBR1, TGFBR2, and combinations thereof using one or more recombinant viral particle. In some embodiments, the one of more viral vectors comprise a recombinant virus selected from a retrovirus, an adenovirus, an adeno-associated virus, a lentivirus, and a herpes simplex virus-1. In some embodiments, the one of more viral vectors comprise a recombinant adeno-associated virus (AAV). In some embodiments, the recombinant AAV is of serotype 5 (AAV5). In some embodiments, the recombinant AAV is of serotype 6 (AAV6). In some embodiments, the one or more viral vectors comprise: a first viral vector comprising a first nucleic acid, in the one or more nucleic acids, encoding the Cas protein; and a second viral vector comprising a second nucleic acid, in the one or more nucleic acids, encoding the at least one guide RNA. In some embodiments, the one or more viral vectors comprise a viral vector comprising a single nucleic acid, wherein the single nucleic acid encodes the Cas9 protein and the at least one guide RNA.

### 1. Adeno-Associated Virus (AAV)

A viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro, and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and Mclaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). The identification of Staphylococcus aureus (SaCas9) and other smaller Cas9 enzymes that can be packaged into adeno-associated viral (AAV) vectors that are highly stable and effective in vivo, easily produced, approved by FDA, and tested in multiple clinical trials, paves new avenues for therapeutic gene editing.

According to particular embodiments, a CRISPR gene-editing system targeting a gene selected from TGFB1, TGFBR1, TGFBR2, and combinations thereof further comprise a recombinant AAV vector. In some embodiments, the CRISPR gene-editing system is encoded by a nucleic acid, wherein the nucleic acid is a recombinant AAV genome. In some embodiments, the AAV vector is selected from an AAVI vector, an AAV2 vector, an AAV3 vector, an AAV4 vector, an AAV5 vector, an AAV6 vector, an AAV7 vector, an AAV8 vector, an AAV9 vector, and an AAV10 vector.

In some aspects, the AAV vector comprises a serotype selected from the group consisting of: AAV1, AAV1 (Y705+731F+T492V), AAV2 (Y444+500+730F+T491V), AAV3 (Y705+731F), AAV4, AAV5, AAV5 (Y436+693+719F), AAV6, AAV6 (VP3 variant Y705F/Y731F/T492V), AAV-7m8, AAV8, AAV8 (Y733F), AAV9, AAV9 (VP3 variant Y731F), AAV10 (Y733F), AAV-ShH10, and AAV-DJ/8. In some aspects, the AAV vector comprises a serotype selected from the group consisting of: AAV1, AAV5, AAV6, AAV6 (Y705F/Y731F/T492V), AAV8, AAV9, and AAV9 (Y731F).

In one aspect, use of the CRISPR gene-editing system further comprising one or more AAV vectors to target TGFB1, TGFBR1 and/or TGFBR2 is therapeutic. In some embodiments, use of the system treats fibrosis and/or scarring. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

### 2. Lentivirus

In some aspects, the viral vector is a lentivirus. In an aspect, the lentivirus is selected from the group consisting of: human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein.

In one aspect, use of the CRISPR gene-editing system further comprising one or more lentivirus vectors to target TGFB1, TGFBR1 and/or TGFBR2 is therapeutic. In some embodiments, use of the system treats fibrosis and/or scarring. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

### B. Lipid Nanoparticles (LNP)

In some embodiments, a CRISPR gene-editing system is delivered by a nanoparticle. Without wishing to be bound by any particular theory, in certain embodiments, nucleic acids, when present in the nanoparticle, are resistant in aqueous solution to degradation with a nuclease. In other embodiments, proteins are protected from protease degradation. In some embodiments, proteins and nucleic acids encapsulated by nanoparticles are capable of penetrating the cellular plasma membrane.

Lipid nanoparticles comprising nucleic acids and their method of preparation is disclosed in at least WO2017/019935, WO2017/049074, WO2017/201346, WO2017/218704, WO2018/006052, WO2018/013525, WO2018/089540, WO2018/119115, WO2018/126084, WO2018/157009, WO2018/170336, WO2018/222890, WO2019/046809, WO2019/089828, WO2020/061284, WO2020/061317, WO2020/081938, WO2020/097511, WO2020/097520, WO2020/097540, WO2020/097548, WO2020/214946, WO2020/219941, WO2020/232276, WO2020/227615, WO2020/061295, WO2021/007278, WO2021/016430, WO2021/021988, EP Patent No. EP 2 972 360, US20200155691, US20200237671, U.S. Pat. Nos. 8,058,069, 8,492,359, 8,822,668, 9,364,435, 9,404,127, 9,504,651, 9,593,077, 9,738,593, 9,868,691, 9,868,692, 9,950,068, 10,138,213, 10,166,298, 10,221,127, 10,238,754, 10,266,485, 10,383,952, 10,730,924, 10,766,852, 11,079,379, 11,141,378 and 11,246,933, which are incorporated herein by reference in their entirety for all purposes.

### Lipid Nanoparticle Compositions

In some embodiments, the largest dimension of a nanoparticle composition is 1 micrometer or shorter (e.g., 1 micrometer, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels. In various embodiments, lipid nanoparticles described herein have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic.

In certain embodiments, the lipid nanoparticles described herein comprise one or more components, including a lipid component,, and (optionally) a structural component. The lipid component comprises lipids selected from ionizable and/or cationic lipids (i.e., lipids that may have a positive or partial positive charge at physiological pH), neutral lipids (e.g., phospholipids, or sphingolipids), and polymer-conjugated lipids (e.g., PEGylated lipids). In some embodiments, the lipid component comprises a single ionizable lipid. In other embodiments, the lipid component comprises 2, 3, 4, 5, 6, 7, 8, 9, 10, 15, or 20 ionizable lipids. In some embodiments, the lipid component comprises a single neutral lipid. In other embodiments, the lipid component comprises 2, 3, 4, 5, 6, 7, 8, 9, 10, 15, or 20 neutral lipids. In some embodiments, the lipid com-ponent comprises a single polymer-conjugated lipid. In other embodiments, the lipid component comprises 2, 3, 4, 5, 6, 7, 8, 9, 10, 15, or 20 polymer-conjugated lipids. In some embodiments, the structural component comprises a single structural lipid. In other embodiments, the structural component comprises 2, 3, 4, 5, 6, 7, 8, 9, 10, 15, or 20 structural lipids. In some embodiments, the lipid component comprises at least one cationic lipid, at least one neutral lipid, and at least one polymer-conjugated lipid. The present disclosure contemplates that the lipid component may comprise any combination of the foregoing constituents.

### Ionizable/Cationic Lipids

In some embodiments, the lipid component comprises an ionizable lipid. In some embodiments, the ionizable lipid is anionic. In other embodiments, the ionizable lipid is a cationic lipid. In some embodiments, the lipid component comprises cationic lipids including, but not limited to, a cationic lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and mixtures thereof.

Non-exhaustive and non-limiting examples of cationic lipids include:

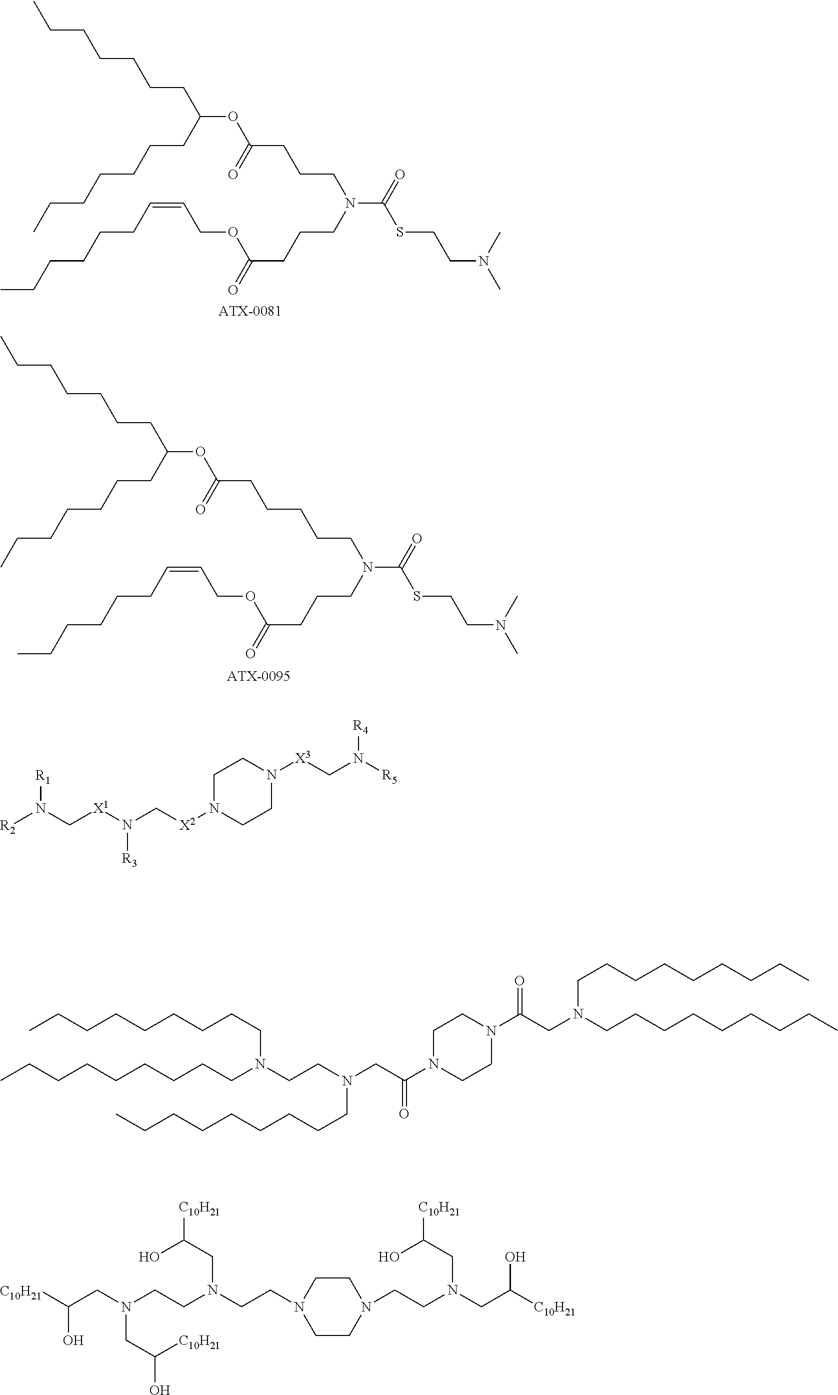

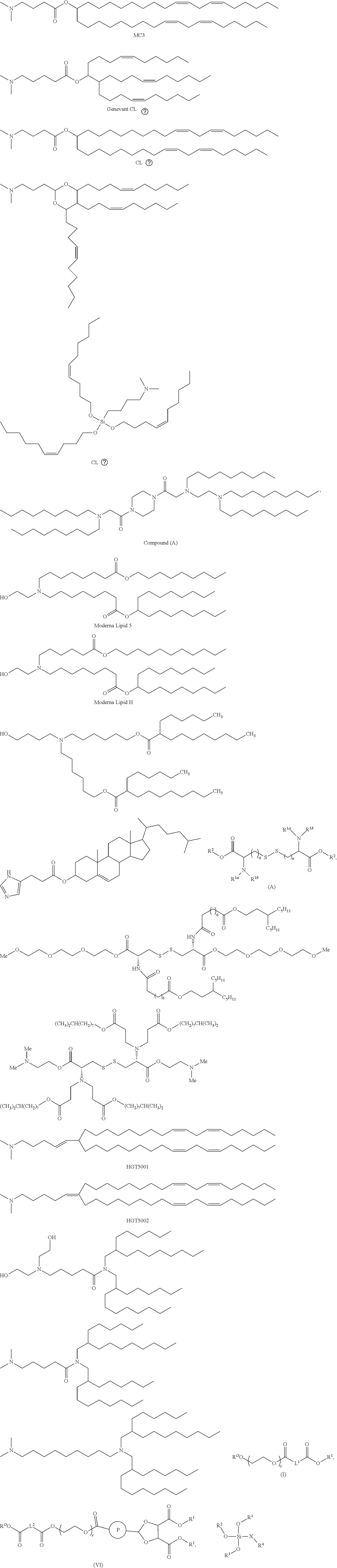

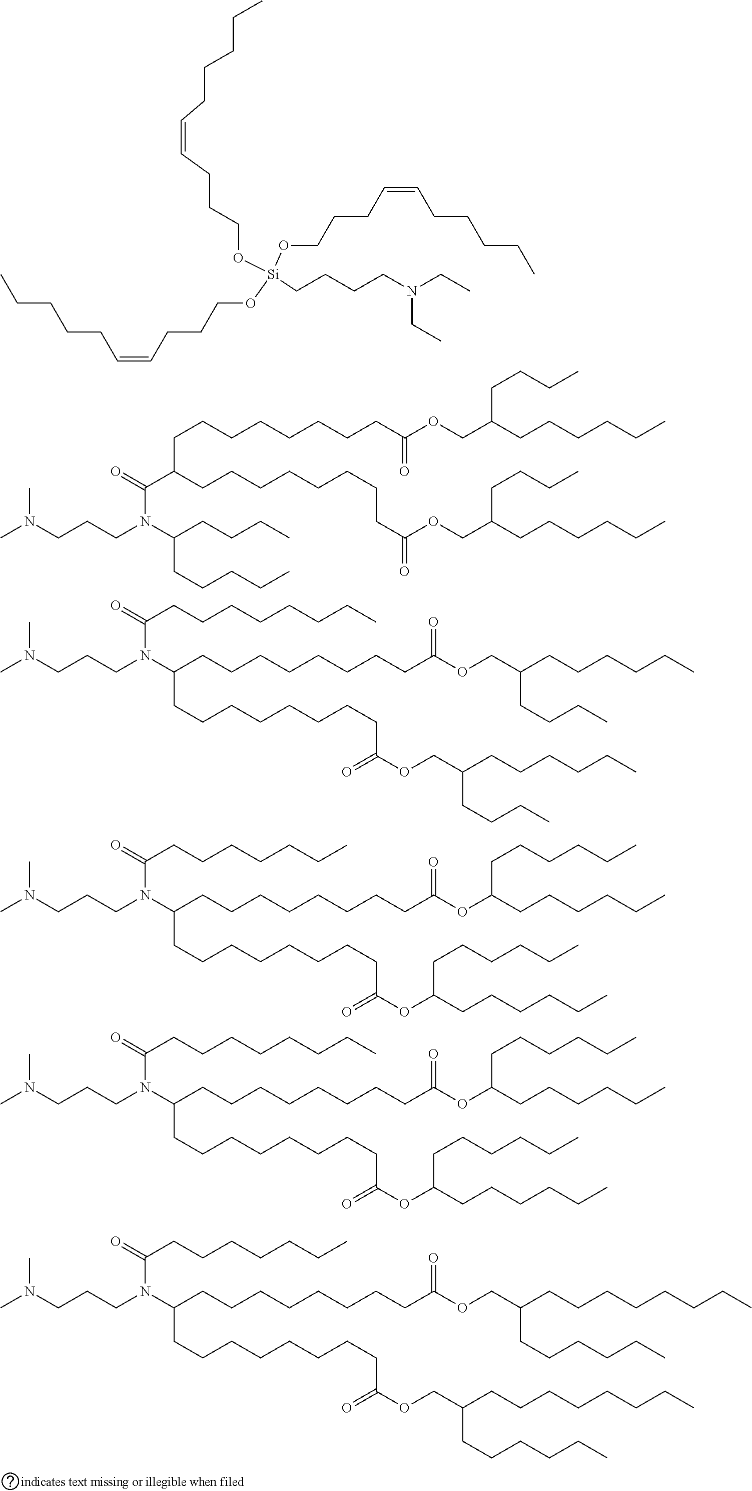

### Neutral Lipids/Phospholipids

In some embodiments, the lipid component further comprises neutral lipids including, but not limited to, a phospholipid selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and mixtures thereof.

### Polymer-conjugated Lipids

In some embodiments, the lipid component further comprises polymer-conjugated lipids, including, but not limited to, a PEGylated lipid selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG2000-c-DMG, PEG-DLPE, PEG-DMPE, PEG-DPPC, PEG-DMA or a PEG-DSPE lipid.

Non-exhaustive and non-limiting examples of PEG lipids include:

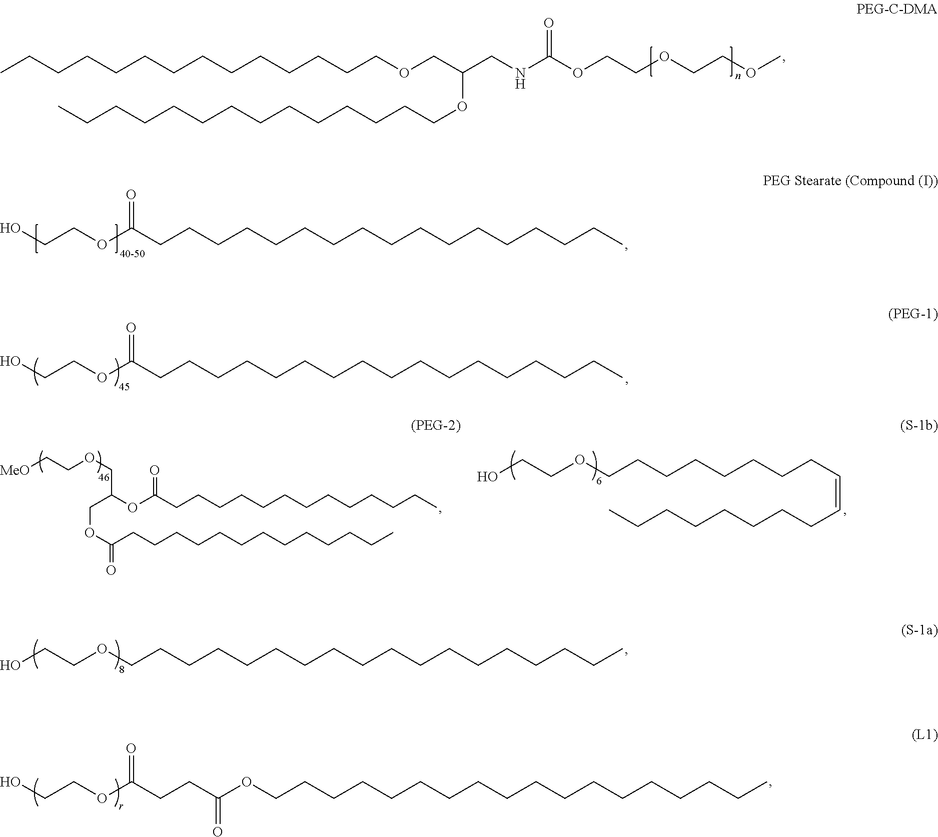

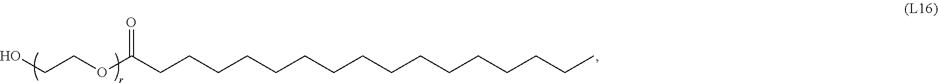

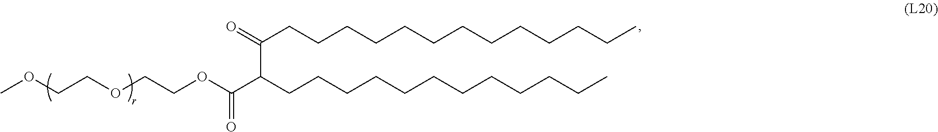

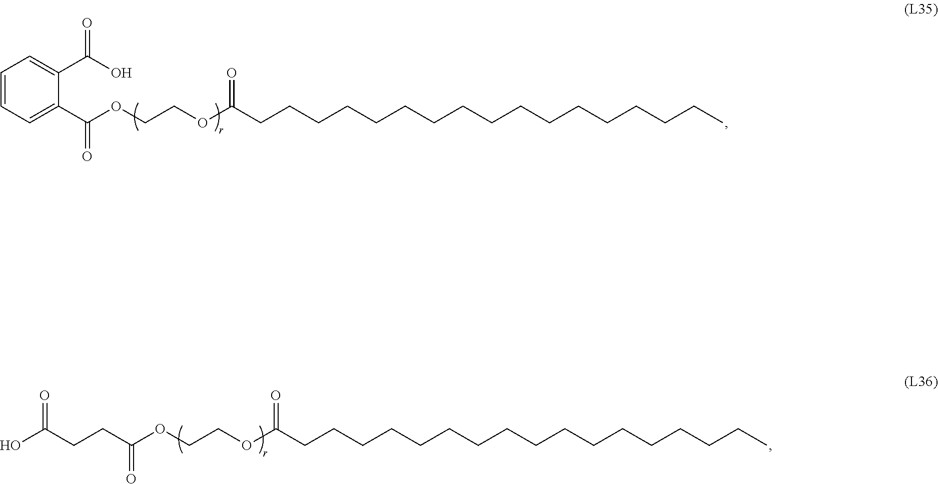

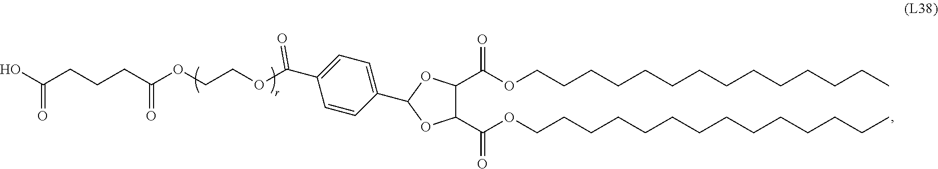

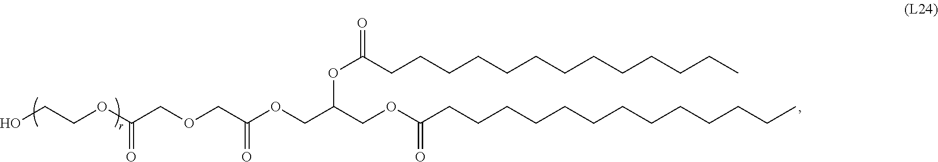

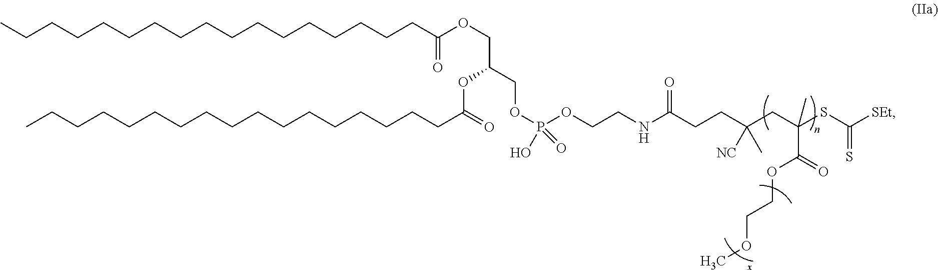

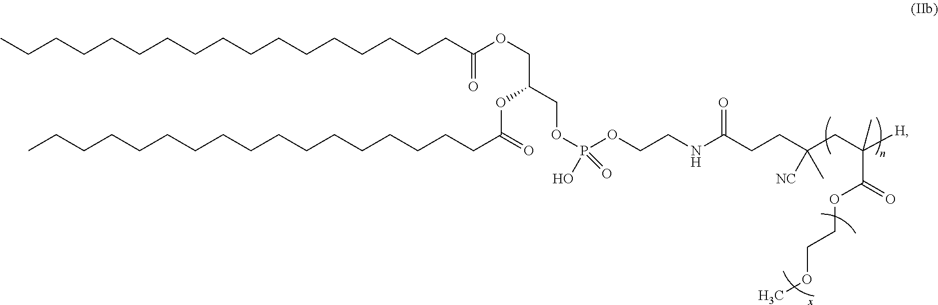

### Structural Lipids/Sterols

In some embodiments, the LNP further comprises a structural component. See generally Patel, S., et al. (2020). Nature Communications, 11 (1), 1-13. In some embodiments, the structural component comprises a sterol including, but not limited to, a sterol selected from the group consisting of cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9, 11-dehydroergosterol, tomatidine, tomatine, a-tocopherol, and mixtures thereof. In other embodiments, the structural lipid includes cholesterol and a corticosteroid (e.g., prednisolone, dexamethasone, prednisone, and hydrocortisone), or a combination thereof. Non-exhaustive and non-limiting examples of structural lipids include:

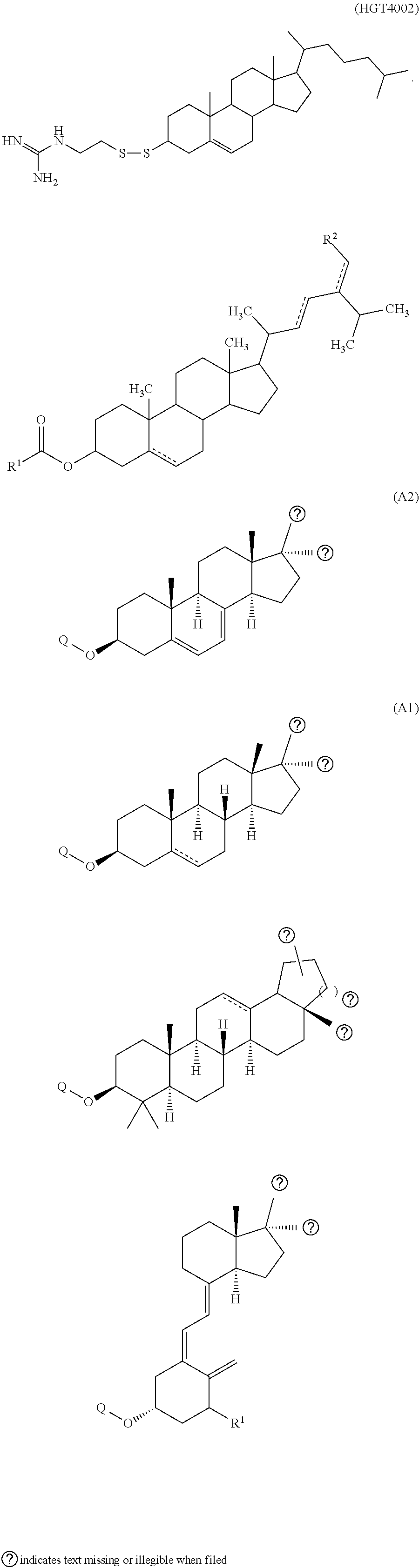

### Formulations

Nanoparticle compositions may include a lipid component and one or more additional components, such as a therapeutic and/or prophylactic. A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

The lipid component of a nanoparticle composition may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the lipid component of a nanoparticle composition includes an ionizable lipid, a phospholipid, a PEG lipid, and a structural lipid. In certain embodiments, the lipid com-ponent of the nanoparticle composition includes about 30 mol % to about 60 mol % ionizable lipid, about 0 mol % to about 30 mol % phospholipid, about 0 mol % to about 10 mol % of PEG lipid, and about 17.5 mol % to about 50 mol % structural lipid, provided that the total mol % does not exceed 100%. In some embodiments, the lipid component of the nanoparticle composition includes about 35 mol % to about 55 mol % compound of ionizable lipid, about 5 mol % to about 25 mol % phospholipid, about 0 mol % to about 10 mol % of PEG lipid, and about 30 mol % to about 40 mol % structural lipid. In a particular embodiment, the lipid component includes about 50 mol % said compound, about 10 mol % phospholipid, about 38.5 mol % structural lipid, and about 1.5 mol % of PEG lipid. In another embodiment, the lipid component includes about 40 mol % said compound, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

In some embodiments, the ionizable lipids comprise between about 20 and about 60 mol % of the lipid component. In other embodiments, the ionizable lipids comprise between about 35 and about 55 mol % of the lipid component. In various embodiments, the ionizable lipids comprise about 20, 21, 22, 23, 24, 25, 26, 27, 28, 29, 30, 35, 40, 45, 50, 55, or 60 mol % of the lipid component.

In some embodiments, the neutral lipids comprise between about 0 and about 30 mol % of the lipid component. In other embodiments, the neutral lipids comprise between about 5 and about 25 mol % of the lipid component. In various embodiments, the neutral lipids comprise about 1, 2, 3, 4, 5, 6, 7, 8, 9, 10, 11, 12, 13, 14, 15, 16, 17, 18, 19, 20, 25, or 30 mol % of the lipid component.

In some embodiments, the polymer-conjugated lipids comprise between about 0 and about 15 mol % of the lipid component. In other embodiments, the polymer-conjugated lipids comprise between about 0.5 and about 10 mol % of the lipid component. In various embodiments, the polymer-conjugated lipids comprise about 0.5, 1, 1.5, 2, 2.5, 3, 3.5, 4, 4.5, 5, 5.5, 6, 6.5, 7, 7.5, 8, 8.5 9, 9.5, 10, or 15 mol % of the lipid component.

In some embodiments, the structural component comprises about 17.5 mol % to about 50 mol % of the lipid component. In other embodiments, the structural component comprises about 30 to about 40 mol % of the lipid component. In various embodiments, the structural component comprises about 17.5, 20, 22.5, 25, 27.5, 30, 31, 32, 33, 34, 35, 36, 37, 38, 39, 40, 41, 42, 43, 44, 45, 46, 47, 48, 49, or 50 mol % of the lipid component.

The structural component may alternatively be expressed as a ratio relative to the lipid component. In some embodiments, the structural component is in a ratio of about 1:1 with the lipid component (sterol: lipids). In other embodiments, the structural component is in a ratio of about 1:5 with the lipid component (sterol: lipids). In various embodiments, the structural component is in a ratio of about 1:1, 1:2, 1:3, 1:4, 1:5, 1:6, 1:7, 1:8, 1:9, 1:10, 1:15, 1:20, or 1:25 with the lipid component (sterol: lipids).

Nanoparticle compositions may be designed for one or more specific applications or targets. For example, a nanoparticle composition may be designed to deliver a therapeutic and/or prophylactic such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic and/or prophylactic included in a nanoparticle composition may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a nanoparticle composition may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian joint. In some embodiments, the nanoparticle is attached or complexed with a targeting moiety for improving delivery to a particular target cell and/or tissue. For example, in some embodiments, the nanoparticle is attached to or complexed with an agent that binds to a cell surface marker of the target cell and/or tissue.

The amount of a therapeutic and/or prophylactic in a nanoparticle composition may depend on the size, composition, desired target and/or application, or other properties of the nanoparticle composition as well as on the properties of the therapeutic and/or prophylactic. For example, the amount of an RNA useful in a nanoparticle composition may depend on the size, sequence, and other characteristics of the RNA. The relative amounts of a therapeutic and/or prophylactic and other elements (e.g., lipids) in a nanoparticle composition may also vary. In some embodiments, the wt/wt ratio of the lipid component to a therapeutic and/or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a nanoparticle composition may, for example, be measured using absorption spectroscopy (e.g., ultraviolet-visible spectroscopy).

In some embodiments, the therapeutic and/or prophylactic comprises a nucleic acid component. In some embodiments, the nucleic acid component comprises RNA including, but not limited to, RNA selected from the group consisting of messenger RNA (mRNA), CRISPR RNA (crRNA), tracrRNA, single-guide RNA (sgRNA), short interfering RNA (siRNA), antisense oligonucleotides (ASO), and mixtures thereof. In other embodiments, the nucleic acid component comprises DNA including, but not limited to, DNA selected from the group consisting of linear DNA, plasmid DNA, antisense oligonucleotide, and mixtures thereof.

In some embodiments, a nanoparticle composition includes one or more RNAs, and the one or more RNAs, lipids, and amounts thereof may be selected to provide a specific N: P ratio. The N: P ratio of the composition refers to the molar ratio of nitrogen atoms in one or more lipids to the number of phosphate groups in an RNA. In general, a lower N: P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N: P ratio may be from about 2:1 to about 8:1. In other embodiments, the N: P ratio is from about 5:1 to about 8:1. For example, the N: P ratio may be about 5.0:1, about 5.5:1, about 5.67:1, about 6.0:1, about 6.5:1, or about 7.0:1. For example, the N: P ratio may be about 5.67:1.

In some embodiments, the nucleic acid component is comprised of a modified nucleic acid. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

In certain embodiments, the present disclosure comprises methods for treating back or spine conditions or disorders. In other embodiments, the present disclosure comprises methods for treating discogenic disorders. In some embodiments, the present disclosure comprises methods for treating localized nociception, inflammation, or morphological changes associated with back or spine conditions or disorders in a subject in need thereof, the method comprising administering a therapeutically effective amount of a CRISPR-Cas composition encapsulated within or associated with a lipid nanoparticle (LNP), wherein the composition comprises one or more non-naturally occurring polynucleotides encoding a Cas9 protein and at least one sgRNA. In some embodiments, LNPs are administered intradiscally. In other embodiments, LNPs are administered epidurally. In some embodiments, LNPs are administered peridiscally. In some embodiments, LNPs are administered perivertebrally.

### Physical Properties

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

The mean size of a nanoparticle composition may be between 10 nm and 1 micrometer, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about-10 mV to about +20 mV, from about-10 mV to about +15 mV, from about-10 mV to about +10 mV, from about-10 mV to about +5 mV, from about-10 mV to about 0 mV, from about-10 mV to about-5 mV, from about-5 mV to about +20 mV, from about-5 mV to about +15 mV, from about-5 mV to about +10 mV, from about-5 mV to about +5 mV, from about-5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic and/or prophylactic in a solution containing the nanoparticle composition before and after breaking up the nanoparticle composition with one or more organic solvents or detergents. Fluorescence may be used to measure the amount of free therapeutic and/or prophylactic (e.g., RNA) in a solution. For the nanoparticle compositions described herein, the encapsulation efficiency of a therapeutic and/or prophylactic may be at least 50%, for example 50%, 55%, 60%, 65%, 70%, 75%, 80%, 85%, 9 0%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In some embodiments, the CRISPR gene-editing system comprises one or more RNA-containing compositions. In some embodiments, the CRISPR gene-editing system further comprises one or more nanoparticles. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanoparticles are lipid nanoparticles (LNP).

In some embodiments, the CRISPR gene-editing system comprises one or more LNPs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNPs comprises a first plurality of LNP encapsulating the RNA-guided nuclease or a nucleic acid encoding an RNA-guided nuclease and a second plurality of LNP encapsulating the at least one guide RNA or a nucleic acid encoding at least one guide RNA.

In some embodiments, the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

In some embodiments, the one or more LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

In some embodiments, the one or more LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

In some embodiments, the one or more LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9, 11-dehydroergosterol, tomatidine, tomatine, a-tocopherol, and a mixture thereof.

In one aspect, use of the CRISPR gene-editing system further comprising one or more LNPs to target TGFB1, TGFBR1 and/or TGFBR2 is therapeutic. In some embodiments, use of the system treats fibrosis and/or scarring. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

### Example LNP Systems

In some embodiments, an LNP system selected from those listed in the table below is used to formulate pharmaceutical compositions described herein:

TABLE 5
LNP Systems (all values are a molar ratio relative to a 100 total)

| | | Neutral/Helper | Steroid | PEGylated |
|---|---|---|---|---|
| | | Lipid | System | Lipid |
| | Ionizable | Component | Component | Component |
| | Lipid Component | (molar ratio or | (molar ratio or | (molar ratio or |
| LNP | (molar ratio or molar | molar ratio | molar ratio | molar ratio |
| System# | ratio range) | range) | range) | range) |
| LNP001 | Dlin-KC2-DMA (42-50) | DSPC (8-10) | (36-46) | (balance to 100) |
| LNP002 | Dlin-KC2-DMA (42-50) | DSPC (10-12) | (36-46) | (balance to 100) |
| LNP003 | Dlin-KC2-DMA (42-50) | DSPC (10 ± 0.5) | (36-46) | (balance to 100) |
| LNP004 | Dlin-KC2-DMA (42-50) | DOPE (8-10) | (36-46) | (balance to 100) |
| LNP005 | Dlin-KC2-DMA (42-50) | DOPE (10-12) | (36-46) | (balance to 100) |
| LNP006 | Dlin-KC2-DMA (42-50) | DOPE (10 ± 0.5) | (36-46) | (balance to 100) |
| LNP007 | Dlin-KC2-DMA (42-50) | DOTMA (8-10) | (36-46) | (balance to 100) |
| LNP008 | Dlin-KC2-DMA (42-50) | DOTMA (10-12) | (36-46) | (balance to 100) |
| LNP009 | Dlin-KC2-DMA (42-50) | DOTMA (10 ± 0.5) | (36-46) | (balance to 100) |
| LNP010 | Dlin-KC2-DMA (42-50) | DPPC (8-10) | (36-46) | (balance to 100) |
| LNP011 | Dlin-KC2-DMA (42-50) | DPPC (10-12) | (36-46) | (balance to 100) |
| LNP012 | Dlin-KC2-DMA (42-50) | DPPC (10 ± 0.5) | (36-46) | (balance to 100) |
| LNP013 | Dlin-KC2-DMA (40-48) | DSPC (8-10) | (36-46) | (balance to 100) |
| LNP014 | Dlin-KC2-DMA (40-48) | DSPC (10-12) | (36-46) | (balance to 100) |
| LNP015 | Dlin-KC2-DMA (40-48) | DSPC (10 ± 0.5) | (36-46) | (balance to 100) |
| LNP016 | Dlin-KC2-DMA (40-48) | DOPE (8-10) | (36-46) | (balance to 100) |
| LNP017 | Dlin-KC2-DMA (40-48) | DOPE (10-12) | (36-46) | (balance to 100) |
| LNP018 | Dlin-KC2-DMA (40-48) | DOPE (10 ± 0.5) | (36-46) | (balance to 100) |
| LNP019 | Dlin-KC2-DMA (40-48) | DOTMA (8-10) | (36-46) | (balance to 100) |
| LNP020 | Dlin-KC2-DMA (40-48) | DOTMA (10-12) | (36-46) | (balance to 100) |
| LNP021 | Dlin-KC2-DMA (40-48) | DOTMA (10 ± 0.5) | (36-46) | (balance to 100) |
| LNP022 | Dlin-KC2-DMA (40-48) | DPPC (8-10) | (36-46) | (balance to 100) |
| LNP023 | Dlin-KC2-DMA (40-48) | DPPC (10-12) | (36-46) | (balance to 100) |
| LNP024 | Dlin-KC2-DMA (40-48) | DPPC (10 ± 0.5) | (36-46) | (balance to 100) |
| LNP025 | Dlin-KC2-DMA (43-45) | DSPC (8-10) | (36-46) | (balance to 100) |
| LNP026 | Dlin-KC2-DMA (43-45) | DSPC (10-12) | (36-46) | (balance to 100) |
| LNP027 | Dlin-KC2-DMA (43-45) | DSPC (10 ± 0.5) | (36-46) | (balance to 100) |
| LNP028 | Dlin-KC2-DMA (43-45) | DOPE (8-10) | (36-46) | (balance to 100) |
| LNP029 | Dlin-KC2-DMA (43-45) | DOPE (10-12) | (36-46) | (balance to 100) |
| LNP030 | Dlin-KC2-DMA (43-45) | DOPE (10 ± 0.5) | (36-46) | (balance to 100) |
| LNP031 | Dlin-KC2-DMA (43-45) | DOTMA (8-10) | (36-46) | (balance to 100) |
| LNP032 | Dlin-KC2-DMA (43-45) | DOTMA (10-12) | (36-46) | (balance to 100) |
| LNP033 | Dlin-KC2-DMA (43-45) | DOTMA (10 ± 0.5) | (36-46) | (balance to 100) |
| LNP034 | Dlin-KC2-DMA (43-45) | DPPC (8-10) | (36-46) | (balance to 100) |
| LNP035 | Dlin-KC2-DMA (43-45) | DPPC (10-12) | (36-46) | (balance to 100) |
| LNP036 | Dlin-KC2-DMA (43-45) | DPPC (10 ± 0.5) | (36-46) | (balance to 100) |
| LNP037 | Dlin-KC2-DMA | DSPC (8-10) | (36-46) | (balance to 100) |
| | (44 ± 0.5) | | | |
| LNP038 | Dlin-KC2-DMA | DSPC (10-12) | (36-46) | (balance to 100) |
| | (44 ± 0.5) | | | |
| LNP039 | Dlin-KC2-DMA | DSPC (10 ± 0.5) | (36-46) | (balance to 100) |
| | (44 ± 0.5) | | | |
| LNP040 | Dlin-KC2-DMA | DOPE (8-10) | (36-46) | (balance to 100) |
| | (44 ± 0.5) | | | |
| LNP041 | Dlin-KC2-DMA | DOPE (10-12) | (36-46) | (balance to 100) |
| | (44 ± 0.5) | | | |
| LNP042 | Dlin-KC2-DMA | DOPE (10 ± 0.5) | (36-46) | (balance to 100) |
| | (44 ± 0.5) | | | |
| LNP043 | Dlin-KC2-DMA | DOTMA (8-10) | (36-46) | (balance to 100) |
| | (44 ± 0.5) | | | |
| LNP044 | Dlin-KC2-DMA | DOTMA (10-12) | (36-46) | (balance to 100) |
| | (44 ± 0.5) | | | |
| LNP045 | Dlin-KC2-DMA | DOTMA (10 ± 0.5) | (36-46) | (balance to 100) |
| | (44 ± 0.5) | | | |
| LNP046 | Dlin-KC2-DMA | DPPC (8-10) | (36-46) | (balance to 100) |
| | (44 ± 0.5) | | | |
| LNP047 | Dlin-KC2-DMA | DPPC (10-12) | (36-46) | (balance to 100) |
| | (44 ± 0.5) | | | |
| LNP048 | Dlin-KC2-DMA | DPPC (10 ± 0.5) | (36-46) | (balance to 100) |
| | (44 ± 0.5) | | | |
| LNP049 | Dlin-MC3-DMA (44-50) | DSPC (8-10) | (36-46) | (balance to 100) |
| LNP050 | Dlin-MC3-DMA (44-50) | DSPC (10-12) | (36-46) | (balance to 100) |
| LNP051 | Dlin-MC3-DMA (44-50) | DSPC (10 ± 0.5) | (36-46) | (balance to 100) |
| LNP052 | Dlin-MC3-DMA (44-50) | DOPE (8-10) | (36-46) | (balance to 100) |
| LNP053 | Dlin-MC3-DMA (44-50) | DOPE (10-12) | (36-46) | (balance to 100) |
| LNP054 | Dlin-MC3-DMA (44-50) | DOPE (10 ± 0.5) | (36-46) | (balance to 100) |
| LNP055 | Dlin-MC3-DMA (44-50) | DOTMA (8-10) | (36-46) | (balance to 100) |
| LNP056 | Dlin-MC3-DMA (44-50) | DOTMA (10-12) | (36-46) | (balance to 100) |
| LNP057 | Dlin-MC3-DMA (44-50) | DOTMA (10 ± 0.5) | (36-46) | (balance to 100) |
| LNP058 | Dlin-MC3-DMA (44-50) | DPPC (8-10) | (36-46) | (balance to 100) |
| LNP059 | Dlin-MC3-DMA (44-50) | DPPC (10-12) | (36-46) | (balance to 100) |
| LNP060 | Dlin-MC3-DMA (44-50) | DPPC (10 ± 0.5) | (36-46) | (balance to 100) |
| LNP061 | Dlin-MC3-DMA (50-54) | DSPC (8-10) | (36-46) | (balance to 100) |
| LNP062 | Dlin-MC3-DMA (50-54) | DSPC (10-12) | (36-46) | (balance to 100) |
| LNP063 | Dlin-MC3-DMA (50-54) | DSPC (10 ± 0.5) | (36-46) | (balance to 100) |
| LNP064 | Dlin-MC3-DMA (50-54) | DOPE (8-10) | (36-46) | (balance to 100) |
| LNP065 | Dlin-MC3-DMA (50-54) | DOPE (10-12) | (36-46) | (balance to 100) |
| LNP066 | Dlin-MC3-DMA (50-54) | DOPE (10 ± 0.5) | (36-46) | (balance to 100) |
| LNP067 | Dlin-MC3-DMA (50-54) | DOTMA (8-10) | (36-46) | (balance to 100) |
| LNP068 | Dlin-MC3-DMA (50-54) | DOTMA (10-12) | (36-46) | (balance to 100) |
| LNP069 | Dlin-MC3-DMA (50-54) | DOTMA (10 ± 0.5) | (36-46) | (balance to 100) |
| LNP070 | Dlin-MC3-DMA (50-54) | DPPC (8-10) | (36-46) | (balance to 100) |
| LNP071 | Dlin-MC3-DMA (50-54) | DPPC (10-12) | (36-46) | (balance to 100) |
| LNP072 | Dlin-MC3-DMA (50-54) | DPPC (10 ± 0.5) | (36-46) | (balance to 100) |
| LNP073 | Dlin-MC3-DMA (49-51) | DSPC (8-10) | (36-46) | (balance to 100) |
| LNP074 | Dlin-MC3-DMA (49-51) | DSPC (10-12) | (36-46) | (balance to 100) |
| LNP075 | Dlin-MC3-DMA (49-51) | DSPC (10 ± 0.5) | (36-46) | (balance to 100) |
| LNP076 | Dlin-MC3-DMA (49-51) | DOPE (8-10) | (36-46) | (balance to 100) |
| LNP077 | Dlin-MC3-DMA (49-51) | DOPE (10-12) | (36-46) | (balance to 100) |
| LNP078 | Dlin-MC3-DMA (49-51) | DOPE (10 ± 0.5) | (36-46) | (balance to 100) |
| LNP079 | Dlin-MC3-DMA (49-51) | DOTMA (8-10) | (36-46) | (balance to 100) |
| LNP080 | Dlin-MC3-DMA (49-51) | DOTMA (10-12) | (36-46) | (balance to 100) |
| LNP081 | Dlin-MC3-DMA (49-51) | DOTMA (10 ± 0.5) | (36-46) | (balance to 100) |
| LNP082 | Dlin-MC3-DMA (49-51) | DPPC (8-10) | (36-46) | (balance to 100) |
| LNP083 | Dlin-MC3-DMA (49-51) | DPPC (10-12) | (36-46) | (balance to 100) |
| LNP084 | Dlin-MC3-DMA (49-51) | DPPC (10 ± 0.5) | (36-46) | (balance to 100) |
| LNP085 | Dlin-MC3-DMA | DSPC (8-10) | (36-46) | (balance to 100) |
| | (50 ± 0.5) | | | |
| LNP086 | Dlin-MC3-DMA | DSPC (10-12) | (36-46) | (balance to 100) |
| | (50 ± 0.5) | | | |
| LNP087 | Dlin-MC3-DMA | DSPC (10 ± 0.5) | (36-46) | (balance to 100) |
| | (50 ± 0.5) | | | |
| LNP088 | Dlin-MC3-DMA | DOPE (8-10) | (36-46) | (balance to 100) |
| | (50 ± 0.5) | | | |
| LNP089 | Dlin-MC3-DMA | DOPE (10-12) | (36-46) | (balance to 100) |
| | (50 ± 0.5) | | | |
| LNP090 | Dlin-MC3-DMA | DOPE (10 ± 0.5) | (36-46) | (balance to 100) |
| | (50 ± 0.5) | | | |
| LNP091 | Dlin-MC3-DMA | DOTMA (8-10) | (36-46) | (balance to 100) |
| | (50 ± 0.5) | | | |
| LNP092 | Dlin-MC3-DMA | DOTMA (10-12) | (36-46) | (balance to 100) |
| | (50 ± 0.5) | | | |
| LNP093 | Dlin-MC3-DMA | DOTMA (10 ± 0.5) | (36-46) | (balance to 100) |
| | (50 ± 0.5) | | | |
| LNP094 | Dlin-MC3-DMA | DPPC (8-10) | (36-46) | (balance to 100) |
| | (50 ± 0.5) | | | |
| LNP095 | Dlin-MC3-DMA | DPPC (10-12) | (36-46) | (balance to 100) |
| | (50 ± 0.5) | | | |
| LNP096 | Dlin-MC3-DMA | DPPC (10 ± 0.5) | (36-46) | (balance to 100) |
| | (50 ± 0.5) | | | |
| LNP097 | SM-102 (44-50) | DSPC (8-10) | (36-46) | (balance to 100) |
| LNP098 | SM-102 (44-50) | DSPC (10-12) | (36-46) | (balance to 100) |
| LNP099 | SM-102 (44-50) | DSPC (10 ± 0.5) | (36-46) | (balance to 100) |
| LNP100 | SM-102 (44-50) | DOPE (8-10) | (36-46) | (balance to 100) |
| LNP101 | SM-102 (44-50) | DOPE (10-12) | (36-46) | (balance to 100) |
| LNP102 | SM-102 (44-50) | DOPE (10 ± 0.5) | (36-46) | (balance to 100) |
| LNP103 | SM-102 (44-50) | DOTMA (8-10) | (36-46) | (balance to 100) |
| LNP104 | SM-102 (44-50) | DOTMA (10-12) | (36-46) | (balance to 100) |
| LNP105 | SM-102 (44-50) | DOTMA (10 ± 0.5) | (36-46) | (balance to 100) |
| LNP106 | SM-102 (44-50) | DPPC (8-10) | (36-46) | (balance to 100) |
| LNP107 | SM-102 (44-50) | DPPC (10-12) | (36-46) | (balance to 100) |
| LNP108 | SM-102 (44-50) | DPPC (10 ± 0.5) | (36-46) | (balance to 100) |
| LNP109 | SM-102 (50-54) | DSPC (8-10) | (36-46) | (balance to 100) |
| LNP110 | SM-102 (50-54) | DSPC (10-12) | (36-46) | (balance to 100) |
| LNP111 | SM-102 (50-54) | DSPC (10 ± 0.5) | (36-46) | (balance to 100) |
| LNP112 | SM-102 (50-54) | DOPE (8-10) | (36-46) | (balance to 100) |
| LNP113 | SM-102 (50-54) | DOPE (10-12) | (36-46) | (balance to 100) |
| LNP114 | SM-102 (50-54) | DOPE (10 ± 0.5) | (36-46) | (balance to 100) |
| LNP115 | SM-102 (50-54) | DOTMA (8-10) | (36-46) | (balance to 100) |
| LNP116 | SM-102 (50-54) | DOTMA (10-12) | (36-46) | (balance to 100) |
| LNP117 | SM-102 (50-54) | DOTMA (10 ± 0.5) | (36-46) | (balance to 100) |
| LNP118 | SM-102 (50-54) | DPPC (8-10) | (36-46) | (balance to 100) |
| LNP119 | SM-102 (50-54) | DPPC (10-12) | (36-46) | (balance to 100) |
| LNP120 | SM-102 (50-54) | DPPC (10 ± 0.5) | (36-46) | (balance to 100) |
| LNP121 | SM-102 (49-51) | DSPC (8-10) | (36-46) | (balance to 100) |
| LNP122 | SM-102 (49-51) | DSPC (10-12) | (36-46) | (balance to 100) |
| LNP123 | SM-102 (49-51) | DSPC (10 ± 0.5) | (36-46) | (balance to 100) |
| LNP124 | SM-102 (49-51) | DOPE (8-10) | (36-46) | (balance to 100) |
| LNP125 | SM-102 (49-51) | DOPE (10-12) | (36-46) | (balance to 100) |
| LNP126 | SM-102 (49-51) | DOPE (10 ± 0.5) | (36-46) | (balance to 100) |
| LNP127 | SM-102 (49-51) | DOTMA (8-10) | (36-46) | (balance to 100) |
| LNP128 | SM-102 (49-51) | DOTMA (10-12) | (36-46) | (balance to 100) |
| LNP129 | SM-102 (49-51) | DOTMA (10 ± 0.5) | (36-46) | (balance to 100) |
| LNP130 | SM-102 (49-51) | DPPC (8-10) | (36-46) | (balance to 100) |
| LNP131 | SM-102 (49-51) | DPPC (10-12) | (36-46) | (balance to 100) |
| LNP132 | SM-102 (49-51) | DPPC (10 ± 0.5) | (36-46) | (balance to 100) |
| LNP133 | SM-102 (50 ± 0.5) | DSPC (8-10) | (36-46) | (balance to 100) |
| LNP134 | SM-102 (50 ± 0.5) | DSPC (10-12) | (36-46) | (balance to 100) |
| LNP135 | SM-102 (50 ± 0.5) | DSPC (10 ± 0.5) | (36-46) | (balance to 100) |
| LNP136 | SM-102 (50 ± 0.5) | DOPE (8-10) | (36-46) | (balance to 100) |
| LNP137 | SM-102 (50 ± 0.5) | DOPE (10-12) | (36-46) | (balance to 100) |
| LNP138 | SM-102 (50 ± 0.5) | DOPE (10 ± 0.5) | (36-46) | (balance to 100) |
| LNP139 | SM-102 (50 ± 0.5) | DOTMA (8-10) | (36-46) | (balance to 100) |
| LNP140 | SM-102 (50 ± 0.5) | DOTMA (10-12) | (36-46) | (balance to 100) |
| LNP141 | SM-102 (50 ± 0.5) | DOTMA (10 ± 0.5) | (36-46) | (balance to 100) |
| LNP142 | SM-102 (50 ± 0.5) | DPPC (8-10) | (36-46) | (balance to 100) |
| LNP143 | SM-102 (50 ± 0.5) | DPPC (10-12) | (36-46) | (balance to 100) |
| LNP144 | SM-102 (50 ± 0.5) | DPPC (10 ± 0.5) | (36-46) | (balance to 100) |
| LNP145 | ALC-0315 (44-50) | DSPC (8-10) | (36-46) | (balance to 100) |
| LNP146 | ALC-0315 (44-50) | DSPC (10-12) | (36-46) | (balance to 100) |
| LNP147 | ALC-0315 (44-50) | DSPC (10 ± 0.5) | (36-46) | (balance to 100) |
| LNP148 | ALC-0315 (44-50) | DOPE (8-10) | (36-46) | (balance to 100) |
| LNP149 | ALC-0315 (44-50) | DOPE (10-12) | (36-46) | (balance to 100) |
| LNP150 | ALC-0315 (44-50) | DOPE (10 ± 0.5) | (36-46) | (balance to 100) |
| LNP151 | ALC-0315 (44-50) | DOTMA (8-10) | (36-46) | (balance to 100) |
| LNP152 | ALC-0315 (44-50) | DOTMA (10-12) | (36-46) | (balance to 100) |
| LNP153 | ALC-0315 (44-50) | DOTMA (10 ± 0.5) | (36-46) | (balance to 100) |
| LNP154 | ALC-0315 (44-50) | DPPC (8-10) | (36-46) | (balance to 100) |
| LNP155 | ALC-0315 (44-50) | DPPC (10-12) | (36-46) | (balance to 100) |
| LNP156 | ALC-0315 (44-50) | DPPC (10 ± 0.5) | (36-46) | (balance to 100) |
| LNP157 | ALC-0315 (50-54) | DSPC (8-10) | (36-46) | (balance to 100) |
| LNP158 | ALC-0315 (50-54) | DSPC (10-12) | (36-46) | (balance to 100) |
| LNP159 | ALC-0315 (50-54) | DSPC (10 ± 0.5) | (36-46) | (balance to 100) |
| LNP160 | ALC-0315 (50-54) | DOPE (8-10) | (36-46) | (balance to 100) |
| LNP161 | ALC-0315 (50-54) | DOPE (10-12) | (36-46) | (balance to 100) |
| LNP162 | ALC-0315 (50-54) | DOPE (10 ± 0.5) | (36-46) | (balance to 100) |
| LNP163 | ALC-0315 (50-54) | DOTMA (8-10) | (36-46) | (balance to 100) |
| LNP164 | ALC-0315 (50-54) | DOTMA (10-12) | (36-46) | (balance to 100) |
| LNP165 | ALC-0315 (50-54) | DOTMA (10 ± 0.5) | (36-46) | (balance to 100) |
| LNP166 | ALC-0315 (50-54) | DPPC (8-10) | (36-46) | (balance to 100) |
| LNP167 | ALC-0315 (50-54) | DPPC (10-12) | (36-46) | (balance to 100) |
| LNP168 | ALC-0315 (50-54) | DPPC (10 ± 0.5) | (36-46) | (balance to 100) |
| LNP169 | ALC-0315 (49-51) | DSPC (8-10) | (36-46) | (balance to 100) |
| LNP170 | ALC-0315 (49-51) | DSPC (10-12) | (36-46) | (balance to 100) |
| LNP171 | ALC-0315 (49-51) | DSPC (10 ± 0.5) | (36-46) | (balance to 100) |
| LNP172 | ALC-0315 (49-51) | DOPE (8-10) | (36-46) | (balance to 100) |
| LNP173 | ALC-0315 (49-51) | DOPE (10-12) | (36-46) | (balance to 100) |
| LNP174 | ALC-0315 (49-51) | DOPE (10 ± 0.5) | (36-46) | (balance to 100) |
| LNP175 | ALC-0315 (49-51) | DOTMA (8-10) | (36-46) | (balance to 100) |
| LNP176 | ALC-0315 (49-51) | DOTMA (10-12) | (36-46) | (balance to 100) |
| LNP177 | ALC-0315 (49-51) | DOTMA (10 ± 0.5) | (36-46) | (balance to 100) |
| LNP178 | ALC-0315 (49-51) | DPPC (8-10) | (36-46) | (balance to 100) |
| LNP179 | ALC-0315 (49-51) | DPPC (10-12) | (36-46) | (balance to 100) |
| LNP180 | ALC-0315 (49-51) | DPPC (10 ± 0.5) | (36-46) | (balance to 100) |
| LNP181 | ALC-0315 (50 ± 0.5) | DSPC (8-10) | (36-46) | (balance to 100) |
| LNP182 | ALC-0315 (50 ± 0.5) | DSPC (10-12) | (36-46) | (balance to 100) |
| LNP183 | ALC-0315 (50 ± 0.5) | DSPC (10 ± 0.5) | (36-46) | (balance to 100) |
| LNP184 | ALC-0315 (50 ± 0.5) | DOPE (8-10) | (36-46) | (balance to 100) |
| LNP185 | ALC-0315 (50 ± 0.5) | DOPE (10-12) | (36-46) | (balance to 100) |
| LNP186 | ALC-0315 (50 ± 0.5) | DOPE (10 ± 0.5) | (36-46) | (balance to 100) |
| LNP187 | ALC-0315 (50 ± 0.5) | DOTMA (8-10) | (36-46) | (balance to 100) |
| LNP188 | ALC-0315 (50 ± 0.5) | DOTMA (10-12) | (36-46) | (balance to 100) |
| LNP189 | ALC-0315 (50 ± 0.5) | DOTMA (10 ± 0.5) | (36-46) | (balance to 100) |
| LNP190 | ALC-0315 (50 ± 0.5) | DPPC (8-10) | (36-46) | (balance to 100) |
| LNP191 | ALC-0315 (50 ± 0.5) | DPPC (10-12) | (36-46) | (balance to 100) |
| LNP192 | ALC-0315 (50 ± 0.5) | DPPC (10 ± 0.5) | (36-46) | (balance to 100) |
| LNP193 | LP01 (42-45) | DSPC (8-10) | (36-46) | (balance to 100) |
| LNP194 | LP01 (42-45) | DSPC (10-12) | (36-46) | (balance to 100) |
| LNP195 | LP01 (42-45) | DSPC (10 ± 0.5) | (36-46) | (balance to 100) |
| LNP196 | LP01 (42-45) | DOPE (8-10) | (36-46) | (balance to 100) |
| LNP197 | LP01 (42-45) | DOPE (10-12) | (36-46) | (balance to 100) |
| LNP198 | LP01 (42-45) | DOPE (10 ± 0.5) | (36-46) | (balance to 100) |
| LNP199 | LP01 (42-45) | DOTMA (8-10) | (36-46) | (balance to 100) |
| LNP200 | LP01 (42-45) | DOTMA (10-12) | (36-46) | (balance to 100) |
| LNP201 | LP01 (42-45) | DOTMA (10 ± 0.5) | (36-46) | (balance to 100) |
| LNP202 | LP01 (42-45) | DPPC (8-10) | (36-46) | (balance to 100) |
| LNP203 | LP01 (42-45) | DPPC (10-12) | (36-46) | (balance to 100) |
| LNP204 | LP01 (42-45) | DPPC (10 ± 0.5) | (36-46) | (balance to 100) |
| LNP205 | LP01 (45-50) | DSPC (8-10) | (36-46) | (balance to 100) |
| LNP206 | LP01 (45-50) | DSPC (10-12) | (36-46) | (balance to 100) |
| LNP207 | LP01 (45-50) | DSPC (10 ± 0.5) | (36-46) | (balance to 100) |
| LNP208 | LP01 (45-50) | DOPE (8-10) | (36-46) | (balance to 100) |
| LNP209 | LP01 (45-50) | DOPE (10-12) | (36-46) | (balance to 100) |
| LNP210 | LP01 (45-50) | DOPE (10 ± 0.5) | (36-46) | (balance to 100) |
| LNP211 | LP01 (45-50) | DOTMA (8-10) | (36-46) | (balance to 100) |
| LNP212 | LP01 (45-50) | DOTMA (10-12) | (36-46) | (balance to 100) |
| LNP213 | LP01 (45-50) | DOTMA (10 ± 0.5) | (36-46) | (balance to 100) |
| LNP214 | LP01 (45-50) | DPPC (8-10) | (36-46) | (balance to 100) |
| LNP215 | LP01 (45-50) | DPPC (10-12) | (36-46) | (balance to 100) |
| LNP216 | LP01 (45-50) | DPPC (10 ± 0.5) | (36-46) | (balance to 100) |
| LNP217 | LP01 (44-46) | DSPC (8-10) | (36-46) | (balance to 100) |
| LNP218 | LP01 (44-46) | DSPC (10-12) | (36-46) | (balance to 100) |
| LNP219 | LP01 (44-46) | DSPC (10 ± 0.5) | (36-46) | (balance to 100) |
| LNP220 | LP01 (44-46) | DOPE (8-10) | (36-46) | (balance to 100) |
| LNP221 | LP01 (44-46) | DOPE (10-12) | (36-46) | (balance to 100) |
| LNP222 | LP01 (44-46) | DOPE (10 ± 0.5) | (36-46) | (balance to 100) |
| LNP223 | LP01 (44-46) | DOTMA (8-10) | (36-46) | (balance to 100) |
| LNP224 | LP01 (44-46) | DOTMA (10-12) | (36-46) | (balance to 100) |
| LNP225 | LP01 (44-46) | DOTMA (10 ± 0.5) | (36-46) | (balance to 100) |
| LNP226 | LP01 (44-46) | DPPC (8-10) | (36-46) | (balance to 100) |
| LNP227 | LP01 (44-46) | DPPC (10-12) | (36-46) | (balance to 100) |
| LNP228 | LP01 (44-46) | DPPC (10 ± 0.5) | (36-46) | (balance to 100) |
| LNP229 | LP01 (45 ± 0.5) | DSPC (8-10) | (36-46) | (balance to 100) |
| LNP230 | LP01 (45 ± 0.5) | DSPC (10-12) | (36-46) | (balance to 100) |
| LNP231 | LP01 (45 ± 0.5) | DSPC (10 ± 0.5) | (36-46) | (balance to 100) |
| LNP232 | LP01 (45 ± 0.5) | DOPE (8-10) | (36-46) | (balance to 100) |
| LNP233 | LP01 (45 ± 0.5) | DOPE (10-12) | (36-46) | (balance to 100) |
| LNP234 | LP01 (45 ± 0.5) | DOPE (10 ± 0.5) | (36-46) | (balance to 100) |
| LNP235 | LP01 (45 ± 0.5) | DOTMA (8-10) | (36-46) | (balance to 100) |
| LNP236 | LP01 (45 ± 0.5) | DOTMA (10-12) | (36-46) | (balance to 100) |
| LNP237 | LP01 (45 ± 0.5) | DOTMA (10 ± 0.5) | (36-46) | (balance to 100) |
| LNP238 | LP01 (45 ± 0.5) | DPPC (8-10) | (36-46) | (balance to 100) |
| LNP239 | LP01 (45 ± 0.5) | DPPC (10-12) | (36-46) | (balance to 100) |
| LNP240 | LP01 (45 ± 0.5) | DPPC (10 ± 0.5) | (36-46) | (balance to 100) |

In some embodiments of any one LNP system LNP001 to LNP240, the LNP system comprises the steroid component in a molar ratio from about 36 to about 46, or from about 38 to about 42. In some embodiments of any one LNP system LNP001 to LNP240, the LNP system comprises the steroid component in a molar ratio of about 36+0.5, about 37±0.5, about 38+0.5, about 39+0.5, about 40+0.5, about 41+0.5, about 42+0.5, about 43+0.5, about 44+0.5, about 45+0.5, or about +0.5. In some embodiments, the LNP system comprises the steroid component in a molar ratio of about 37, about 38, about 39, about 40, about 41, or about 42. In some embodiments of any one LNP system LNP001 to LNP240, the steroid component comprises cholesterol. In some embodiments of any one LNP system LNP001 to LNP240, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000.

LNPs described herein typically include a Biodegradable Ionizable Lipid, a Helper phospholipid, Cholesterol (with the potentially addition of dexamethasone or other steroids), and a PEG2000 component. Molar Lipid Ratio described herein are ratio of ionizable lipid: helper lipid:cholesterol [: Dexamethasone]: PEG.

Modifying molar lipid ratios: Without wishing to be bound by any particular theory, when altering the molar lipid ratio of ionizable lipids, the amount of cholesterol or helper lipid would either be supplemented or decreased. Typically, ionizable lipids are kept at a set ratio, while helper lipids, cholesterol and PEG content are varied. If you were to alter the amount of PEG, you would subsequently adjust the amount of primarily cholesterol and sometimes the helper lipid. If altering the helper lipid, the amount of cholesterol in combination with this is adjusted. Dexamethasone, or another similar steroid in the LNP structure directly replaces some of the cholesterol due to similar chemical structures/function in the LNP.

Typical ionizable lipids are: Dlin-KC2-DMA: molar ratios between 42-50, e.g., and without limitation, 44; Dlin-MC3-DMA: molar ratios between 44-50, e.g., and without limitation, 50; SM-102 (Lipid H): molar ratios between 44-50, e.g., and without limitation, 50; ALC-0315: molar ratios between 44-50, e.g., and without limitation, 50; LP01 (LP000001): molar ratios between 42-50, e.g., and without limitation, 45. Helper Lipids can have molar ratios between 9-11, e.g., and without limitation, 10. Helper lipids include DSPC, DOPE, DOTMA, and DPPC. PEG2000 lipids have typical molar ratios between about 1.5 and about 2.5. These include, without limitation, DMG-PEG, DMG-C-PEG, DMG-PEG2000, DMG-C-PEG2000, DSG-PEG2000, and C14,16,18-PEG2000. Cholesterol molar lipid ratios are typically between about 36 and about 46. Dexamethasone or similar steroids supplement a portion of the molar lipid ratio of cholesterol, for example, and without limitation, a ratio of cholesterol: dexamethasone (C: D) of 9:1. Molar lipid ratio ranges of Cholesterol can be, without limitation, between about 8 and about 10 and Dexamethasone, without limitation, between about 0.1 and about 2. Without wishing to be bound by any particular theory, modifications or replacements for cholesterol can be considered, for example, and without limitation, hydroxy- or alkyl modification (e.g., to improve mRNA delivery) or substitution with potentially therapeutic moieties such as anti-inflammatory steroids. In some embodiments, Molar N/P ratios cab be between 1-8. Without wishing to be bound by any particular, it is believed that to reduce inflammatory response of LNPs in vivo, the ideal range is 1-5 for N/P, however, N/P ratios of 6-8 are tested.

In some embodiments, LNP formulations described herein include LP01: DSPC: Cholesterol:DMG-PEG2000, LP01: DSPC: Cholesterol: Dexamethasone: DMG-PEG2000, MC3: DSPC: Cholesterol:DMG-PEG2000, MC3: DSPC: Cholesterol: Dexamethasone: DMG-PEG2000, MC3: DSPC: Cholesterol: DMG-C-PEG2000, MC3: DSPC: Cholesterol: Dexamethasone: DMG-C-PEG2000, SM-102: DSPC: Cholesterol:DMG-PEG2000, SM-102: DSPC: Cholesterol: Dexamethasone: DMG-PEG2000, ALC-0315: DSPC: Cholesterol:DMG-PEG2000, ALC-0315: DSPC: Cholesterol: Dexamethasone: DMG-PEG2000. In some embodiments, molar ratios of formulations described herein include 50:10:38.5:1.5; 45:9:44:2.

Biophysical Assays: LNPs efficiency to encapsulate nucleic acids, meet sizing criteria and are homogeneity, stability after freeze-thaw cycles. Payloads encapsulated: GFP mRNA, Luciferase mRNA, and our CRISPR/cas9 therapeutic (sgRNA and cas9 mRNA).

TABLE 6
LNPs of various formulations created and screened.

| | | | RNA |
|---|---|---|---|
| ID | LNP Composition | Ratio | Payload |
| LNP 02 | MC3:DSPC:Cholesterol:DMG- | 50:10:38.5:1.5 | OCB02::cas9 |
| | PEG2000 | | mRNA |
| LNP 03 | MC3:DSPC:Cholesterol:Dexamethasone:DMG- | 50:10:34.65:3.85:1.5 | OCB02::cas9 |
| | PEG2000 | | mRNA |
| LNP 04 | MC3:DSPC:Cholesterol:DMG- | 50:10:38.5:1.5 | OCB02::cas9 |
| | C-PEG2000 | | mRNA |
| LNP 05 | KC2:DPPC:Cholesterol:C16- | 47.4:10.1:39.5:4 | Luciferase |
| | C-PEG2000 | | mRNA |
| LNP 07 | MC3:DPSC:Cholesterol:DMG- | 50:10:38.5:1.5 | Luciferase |
| | PEG2000 | | mRNA |

### Additional Notes on LNP Components:

Ionizable lipid (titratable charge): Contains a tertiary amine, making this positively charged at an acidic pH, neutrally charged at a physiological pH. Become protonated after endosomal uptake into the cytosol. See below for specifications on different types of ionizable lipids.

Helper phospholipid: anionic endosomal phospholipids that interact with protonated ionizable lipids to form cone shaped ion pairs that enhance cell membrane fusion and disruption, endosomal escape, and cargo release into cell cytosol.

Cholesterol: maintains nanoparticle membrane integrity, assists encapsulation of nucleic acids, and enhances circulation by reducing surface bound proteins.

Dexamethasone or similar steroids: provide an anti-inflammatory component to the LNPs, which decrease immunogenicity and increase transfection rates, particularly in vivo.

PEGylated lipid (PEG2000): improves circulation half-life, reduces aggregation of LNPs, and reduces interactions with serum proteins such as opsonins, enhancing stability in vivo. Additionally assists particle stability, particle sizing, and biodistribution.

### Ionizable Lipids:

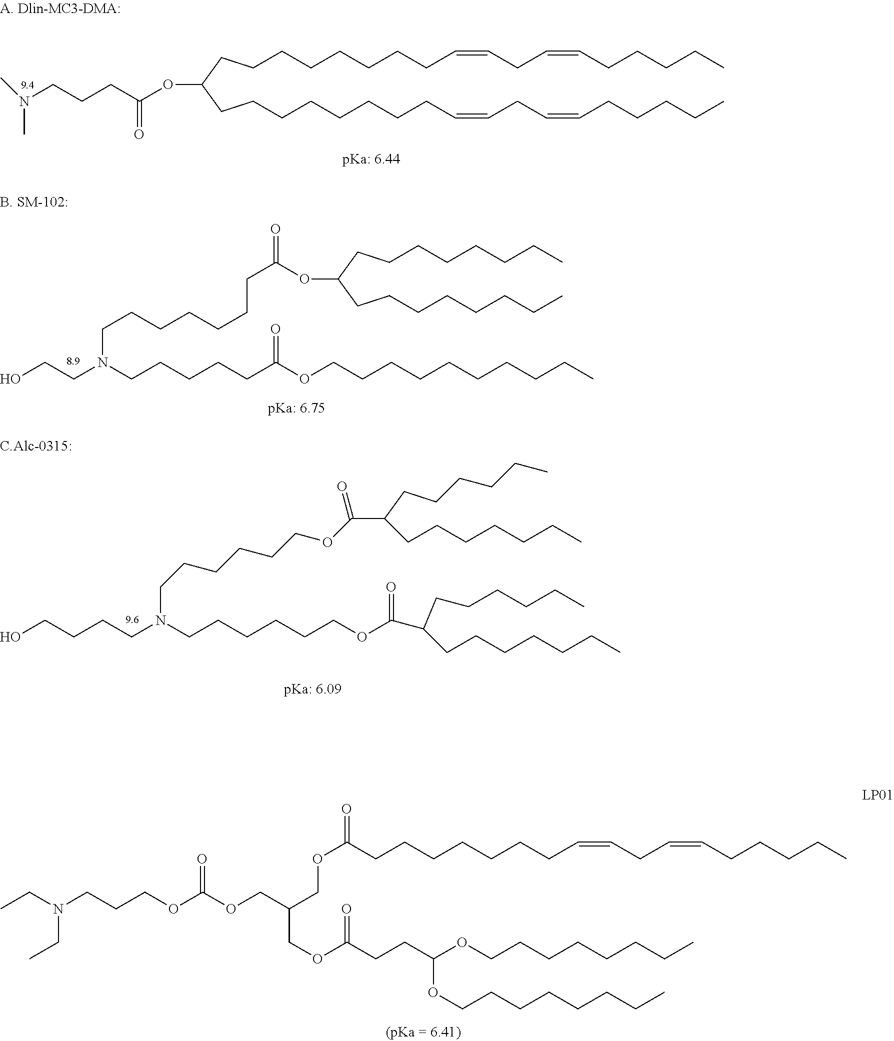

Cationic lipids: such as DODMA, DOTMA, and DOTAP have a quaternary amine group, which leaves them permanently positively charged. Use of this alone was in the past proven to have poor circulation and increased toxicity in vivo. Led to the development of ionizable lipids.

Ionizable lipids are characterised by a replacing quaternary amine to be tertiary, which enables them to be pH dependent, i.e., is neutrally charged at physiological pH and becomes positively charged (protonated) at acidic pH.

This increases circulation half-life and reduces toxicity in vivo. pKa of ionizable lipid drives performance in vivo. pKas between 6-6.7 have been shown to be optimal for delivery of RNA therapeutics. However, the relative pKa of combination of all components in LNP influences LNP transfection-tertiary amine, quaternary amino and hydroxyl group from ionizable lipid, helper lipids and cholesterol all alter relative pKa due to proximity between headgroups. This subsequently affects overall surface charge.

PH dependency enables efficient encapsulation of RNAs in acidic buffer as well as assists RNA release once uptaken by cells.

Further, these lipid pairs form an inverted hexagonal HII phase, which assists membrane disruption, endosomal escape, and subsequent payload release into cytosol of cells.

Albertson et al., 2022, Lipid packing theory showing relationship between amphipathic compounds and geometry once self-assembled. Proposed mechanism by which ionizable lipids mediate endosomal disruption (also known as molecular shape hypothesis). https://www.ncbi.nlm.nih.gov/pmc/articles/PMC9250827

MC3, ALC-0135, SM-102 all have tertiary amines (making them ionizable), no stereo centres, and ester linkers. ALC-0135 and SM-102 have been improved upon MC3 to incorporate additional ester bonds (to assist with biodegradability and reduce bioaccumulation).

Unsaturation of linear tails increases delivery efficiency and fluidity in ionizable lipids, i.e., this enables bilayer lipids to form a non-bilayer phase that will increase membrane disruption and payload release. Additional research explored branching the tails of ionizable lipids, which further increased potency when delivering mRNA therapeutics due to an increase in protonation of ionizable lipids at endosomal pH and cross-section of lipid tails. This modification also led to an increase in cone-shape structure, which facilitates membrane disruption, endosomal escape, and payload release into the cytosol (Albertson et al., 2022).

LP01 incorporates increase in ester bonds as well as branching of tails, which has been shown to be an efficient and safe delivery system for in-vivo gene editing in animal models. LP01 has less liver bioaccumulation and fewer safety risks.

### Example LP01 LNP Formulations

In some embodiments, a composition is provided comprising lipid nanoparticles (LNP) comprising (i) an RNA-guided nuclease or a nucleic acid encoding an RNA-guided nuclease, and (ii) at least one guide RNA or a nucleic acid encoding at least one guide RNA targeting a gene, where the LNP comprises LP01.

In some embodiments, the LNP is composed of the following structural component: an LP01 ionizable lipid, a neutral/helper lipid component, a steroid system component, and a pegylated lipid component. In some such embodiments of the LP01-based LNPs, the neutral/helper lipid component comprises DSPC. In some such embodiments of the LP01-based LNPs, the steroid system component comprises cholesterol. In some such embodiments of the LP01-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the LP01-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): LP01 (40-50), neutral/helper lipid component (6-12), steroid system component (39-49), and a pegylated lipid component (1-3). In some such embodiments, the neutral/helper lipid component comprises DSPC. In some such embodiments of the LP01-based LNPs, the steroid system component comprises cholesterol. In some such embodiments of the LP01-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the LP01-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): LP01 (41-49), neutral/helper lipid component (6.5-11.5), steroid system component (40-48), and a pegylated lipid component (1.25-2.75). In some such embodiments, the neutral/helper lipid component comprises DSPC. In some such embodiments of the LP01-based LNPs, the steroid system component comprises cholesterol. In some such embodiments of the LP01-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the LP01-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): LP01 (42-48), neutral/helper lipid component (7-11), steroid system component (41-47), and a pegylated lipid component (1.5-2.5). In some such embodiments, the neutral/helper lipid component comprises DSPC. In some such embodiments of the LP01-based LNPs, the steroid system component comprises cholesterol. In some such embodiments of the LP01-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the LP01-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): LP01 (43-47), neutral/helper lipid component (7.5-10.5), steroid system component (42-46), and a pegylated lipid component (1.5-2.5). In some such embodiments, the neutral/helper lipid component comprises DSPC. In some such embodiments of the LP01-based LNPs, the steroid system component comprises cholesterol. In some such embodiments of the LP01-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the LP01-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): LP01 (44-46), neutral/helper lipid component (8-10), steroid system component (43-45), and a pegylated lipid component (1.5-2.5). In some such embodiments, the neutral/helper lipid component comprises DSPC. In some such embodiments of the LP01-based LNPs, the steroid system component comprises cholesterol. In some such embodiments of the LP01-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the LP01-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): LP01 (45), neutral/helper lipid component (9), steroid system component (44), and a pegylated lipid component (2). In some such embodiments, the neutral/helper lipid component comprises DSPC. In some such embodiments of the LP01-based LNPs, the steroid system component comprises cholesterol. In some such embodiments of the LP01-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the LP01-based LNPs, the steroid system component comprises cholesterol and a glucocorticoid steroid. In some embodiments, the glucocorticoid steroid is dexamethasone.

### Example SM-102 LNP Formulations

In some embodiments, a composition is provided comprising lipid nanoparticles (LNP) comprising (i) an RNA-guided nuclease or a nucleic acid encoding an RNA-guided nuclease, and (ii) at least one guide RNA or a nucleic acid encoding at least one guide RNA targeting a gene, where the LNP comprises SM-102.

In some embodiments, the LNP is composed of the following structural component: an SM-102 ionizable lipid, a neutral/helper lipid component, a steroid system component, and a pegylated lipid component. In some such embodiments of the SM-102-based LNPs, the neutral/helper lipid component comprises DSPC. In some such embodiments of the SM-102-based LNPs, the steroid system component comprises cholesterol. In some such embodiments of the SM-102-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the SM-102-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): SM-102 (45-55), neutral/helper lipid component (7-13), steroid system component (33.5-43.5), and a pegylated lipid component (0.5-2.5). In some such embodiments, the neutral/helper lipid component comprises DSPC. In some such embodiments of the SM-102-based LNPs, the steroid system component comprises cholesterol. In some such embodiments of the SM-102-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the SM-102-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): SM-102 (46-54), neutral/helper lipid component (7.5-12.5), steroid system component (34.5-42.5), and a pegylated lipid component (0.75-2.25). In some such embodiments, the neutral/helper lipid component comprises DSPC. In some such embodiments of the SM-102-based LNPs, the steroid system component comprises cholesterol. In some such embodiments of the SM-102-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the SM-102-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): SM-102 (47-53), neutral/helper lipid component (8-12), steroid system component (35.5-41.5), and a pegylated lipid component (1-2). In some such embodiments, the neutral/helper lipid component comprises DSPC. In some such embodiments of the SM-102-based LNPs, the steroid system component comprises cholesterol. In some such embodiments of the SM-102-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the SM-102-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): SM-102 (48-52), neutral/helper lipid component (8.5-11.5), steroid system component (36.5-40.5), and a pegylated lipid component (1-2). In some such embodiments, the neutral/helper lipid component comprises DSPC. In some such embodiments of the SM-102-based LNPs, the steroid system component comprises cholesterol. In some such embodiments of the SM-102-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the SM-102-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): SM-102 (49-51), neutral/helper lipid component (9-11), steroid system component (37.5-39.5), and a pegylated lipid component (1-2). In some such embodiments, the neutral/helper lipid component comprises DSPC. In some such embodiments of the SM-102-based LNPs, the steroid system component comprises cholesterol. In some such embodiments of the SM-102-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the SM-102-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): SM-102 (50), neutral/helper lipid component (10), steroid system component (38.5), and a pegylated lipid component (1.5). In some such embodiments, the neutral/helper lipid component comprises DSPC. In some such embodiments of the SM-102-based LNPs, the steroid system component comprises cholesterol. In some such embodiments of the SM-102-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the SM-102-based LNPs, the steroid system component comprises cholesterol and a glucocorticoid steroid. In some embodiments, the glucocorticoid steroid is dexamethasone.

### Example ALC0315 LNP Formulations

In some embodiments, a composition is provided comprising lipid nanoparticles (LNP) comprising (i) an RNA-guided nuclease or a nucleic acid encoding an RNA-guided nuclease, and (ii) at least one guide RNA or a nucleic acid encoding at least one guide RNA targeting a gene, where the LNP comprises ALC0315.

In some embodiments, the LNP is composed of the following structural component: an ALC0315 ionizable lipid, a neutral/helper lipid component, a steroid system component, and a pegylated lipid component. In some such embodiments of the ALC0315-based LNPs, the neutral/helper lipid component comprises DSPC. In some such embodiments of the ALC0315-based LNPs, the steroid system component comprises cholesterol. In some such embodiments of the ALC0315-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the ALC0315-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): ALC0315 (45-55), neutral/helper lipid component (7-13), steroid system component (33.5-43.5), and a pegylated lipid component (0.5-2.5). In some such embodiments, the neutral/helper lipid component comprises DSPC. In some such embodiments of the ALC0315-based LNPs, the steroid system component comprises cholesterol. In some such embodiments of the ALC0315-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the ALC0315-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): ALC0315 (46-54), neutral/helper lipid component (7.5-12.5), steroid system component (34.5-42.5), and a pegylated lipid component (0.75-2.25). In some such embodiments, the neutral/helper lipid component comprises DSPC. In some such embodiments of the ALC0315-based LNPs, the steroid system component comprises cholesterol. In some such embodiments of the ALC0315-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the ALC0315-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): ALC0315 (47-53), neutral/helper lipid component (8-12), steroid system component (35.5-41.5), and a pegylated lipid component (1-2). In some such embodiments, the neutral/helper lipid component comprises DSPC. In some such embodiments of the ALC0315-based LNPs, the steroid system component comprises cholesterol. In some such embodiments of the ALC0315-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the ALC0315-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): ALC0315 (48-52), neutral/helper lipid component (8.5-11.5), steroid system component (36.5-40.5), and a pegylated lipid component (1-2). In some such embodiments, the neutral/helper lipid component comprises DSPC. In some such embodiments of the ALC0315-based LNPs, the steroid system component comprises cholesterol. In some such embodiments of the ALC0315-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the ALC0315-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): ALC0315 (49-51), neutral/helper lipid component (9-11), steroid system component (37.5-39.5), and a pegylated lipid component (1-2). In some such embodiments, the neutral/helper lipid component comprises DSPC. In some such embodiments of the ALC0315-based LNPs, the steroid system component comprises cholesterol. In some such embodiments of the ALC0315-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the ALC0315-based LNPs, the steroid system component comprises cholesterol and a glucocorticoid steroid. In some embodiments, the glucocorticoid steroid is dexamethasone.

In some embodiments, the structural components of the LNP are present in the following relative molar amounts (provided in parenthesis following the component): ALC0315 (50), neutral/helper lipid component (10), steroid system component (38.5), and a pegylated lipid component (1.5). In some such embodiments, the neutral/helper lipid component comprises DSPC. In some such embodiments of the ALC0315-based LNPs, the steroid system component comprises cholesterol. In some such embodiments of the ALC0315-based LNPs, the pegylated lipid component comprises a pegylated myristoyl diglyceride (DMG-PEG; 1,2-Dimyristoyl-sn-glycero-3-methoxypolyethylene glycol). In some embodiments, the DMG-PEG is DMG-PEG2000. In some such embodiments of the ALC0315-based LNPs, the steroid system component comprises cholesterol and a glucocorticoid steroid. In some embodiments, the glucocorticoid steroid is dexamethasone.

### C. Virus-Like Particles

In one aspect, the present disclosure encompasses means for delivering a CRISPR gene-editing system to a mammalian cell via a virus-like particle (VLP). In some embodiments, a CRISPR gene-editing system is delivered by a VLP. Without wishing to be bound by any particular theory, in certain embodiments, nucleic acids, when present in the particle, are resistant in aqueous solution to degradation with a nuclease. In other embodiments, proteins are protected from protease degradation while present in the particle. In some embodiments, proteins and nucleic acids encapsulated by VLPs are capable of penetrating the cellular plasma membrane.

In some embodiments, the CRISPR gene-editing system comprises one or more RNA-containing compositions. In some embodiments, the CRISPR gene-editing system further comprises one or more VLPs. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, the CRISPR gene-editing system comprises one or more virus-like particles collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more virus-like particles comprises a first plurality of virus-like particles encapsulating the RNA-guided nuclease or a nucleic acid encoding an RNA-guided nuclease and a second plurality of virus-like particles encapsulating the at least one guide RNA or a nucleic acid encoding at least one guide RNA.

In one aspect, use of the CRISPR gene-editing system further comprising one or more VLPs to target TGFB1, TGFBR1 and/or TGFBR2 is therapeutic. In some embodiments, use of the system treats fibrosis and/or scarring. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

### D. Miscellaneous Modes of Delivery

### 1. Liposomes

In some embodiments, nucleic acids encoding a CRISPR gene-editing system targeting a gene selected from TGFB1, TGFBR1, TGFBR2, and combinations thereof (e.g., Cas9 or gRNA) are entrapped in liposomes bearing positive charges on their surface (e.g., lipofectins), which can be tagged with antibodies against cell surface antigens of the target cells. These delivery vehicles can also be used to deliver Cas9 protein/gRNA complexes.

In some embodiments, the CRISPR gene-editing system comprises one or more RNA-containing compositions. In some embodiments, the CRISPR gene-editing system further comprises one or more liposomes. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, wherein the composition comprises one or more liposomes collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more liposomes comprises a first plurality of liposomes encapsulating the RNA-guided nuclease or a nucleic acid encoding an RNA-guided nuclease and a second plurality of liposomes encapsulating the at least one guide RNA or a nucleic acid encoding at least one guide RNA.

In one aspect, use of the CRISPR gene-editing system further comprising one or more liposomes to target TGFB1, TGFBR1 and/or TGFBR2 is therapeutic. In some embodiments, use of the system treats fibrosis and/or scarring. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

### 2. Lipid Nanocrystals (LNC)

In one aspect, the present disclosure encompasses means for delivering a CRISPR gene-editing system to a mammalian cell via a lipid nanocrystal (LNC). In some embodiments, a CRISPR gene-editing system is delivered by a LNC. Without wishing to be bound by any particular theory, in certain embodiments, nucleic acids, when present in the nanocrystal, are resistant in aqueous solution to degradation with a nuclease. In other embodiments, proteins are protected from protease degradation while present in the nanocrystal. In some embodiments, proteins and nucleic acids encapsulated by nanocrystal are capable of penetrating the cellular plasma membrane.

In some embodiments, the CRISPR gene-editing system comprises one or more RNA-containing compositions. In some embodiments, the CRISPR gene-editing system further comprises one or more nanocrystals. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanocrystals are lipid nanocrystals (LNC).

In some embodiments, the CRISPR gene-editing system comprises one or more LNCs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNCs comprises a first plurality of LNC encapsulating the RNA-guided nuclease or a nucleic acid encoding an RNA-guided nuclease and a second plurality of LNC encapsulating the at least one guide RNA or a nucleic acid encoding at least one guide RNA.

In one aspect, use of the CRISPR gene-editing system further comprising one or more LNCs to target TGFB1, TGFBR1 and/or TGFBR2 is therapeutic. In some embodiments, use of the system treats fibrosis and/or scarring. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

### VI. Pharmaceutical Compositions

In one aspect, the present disclosure encompasses pharmaceutical compositions comprising a CRISPR gene-editing system for treatment of a mammal in need thereof. In some embodiments, the CRISPR gene-editing system targets a gene selected from TGFB1, TGFBR1, TGFBR2, and combinations thereof. In some embodiments, the mammal is selected from a human, a dog, a horse, and a cat.

### A. TGFB1

In some embodiments, the pharmaceutical composition comprising a CRISPR gene-editing system targets TGFB1. In some embodiments, the CRISPR gene-editing system targeting TGFB1 is delivered to a mammalian cell via viral vector. In some embodiments, the CRISPR gene-editing system targeting TGFB1 is delivered to a mammalian cell via an AAV vector. In some embodiments, the CRISPR gene-editing system targeting TGFB1 is delivered to a mammalian cell via a lentiviral vector. In some embodiments, the CRISPR gene-editing system targeting TGFB1 is delivered to a mammalian cell via a lipid nanoparticle. In some embodiments, the CRISPR gene-editing system targeting TGFB1 is delivered to a mammalian cell via a virus-like particle. In some embodiments, the CRISPR gene-editing system targeting TGFB1 is delivered to a mammalian cell via a liposome. In some embodiments, the CRISPR gene-editing system targeting TGFB1 is delivered to a mammalian cell via a lipid nanocrystal. In some embodiments, the CRISPR gene-editing system targeting TGFB1 is delivered to a mammalian cell via a polymeric nanoparticle. For a review on polymeric nanoparticles, see Zielińska A, et al., “Polymeric Nanoparticles: Production, Characterization, Toxicology and Ecotoxicology,” Molecules, 25 (16): 3731 (2020), the disclosure of which is incorporated herein by reference in its entirety for all purposes. In some embodiments, the CRISPR gene-editing system targeting TGFB1 is delivered to a mammalian cell via an exosome. For a review on exosomes, see Dimik M, et al., “The exosome: a review of current therapeutic roles and capabilities in human reproduction,” Drug Deliv. Transl. Res., 13 (2): 473-502 (2023), the disclosure of which is incorporated herein by reference in its entirety for all purposes.

In various embodiments, the pharmaceutical composition comprising a CRISPR gene-editing system that targets the TGFB1 gene is used in a method of treating a mammal in need thereof. In some embodiments, the method treats fibrosis and/or scarring. In some embodiments, the method treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, the method treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

### 1. Treatment of Musculoskeletal Disorders by Targeting a Transforming Growth Factor Beta Gene

In one aspect, methods and pharmaceutical compositions are provided for treating a musculoskeletal disorder using a CRISPR pharmaceutical. In one aspect, methods and pharmaceutical compositions are provided for treating a musculoskeletal disorder using a CRISPR pharmaceutical composition that targets a Transforming Growth Factor Beta gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta gene. In some embodiments, the mRNA encoding the RNA-guided nuclease and the at least one gRNA are packaged in a lipid nanoparticle (LNP). In some embodiments, the musculoskeletal disorder is Loeys-Dietz Syndrome, Osteoarthrosis, Marfan Syndrome, Aortic aneurysm (familial thoracic 3), or Craniofacial abnormalities is treated with a CRISPR pharmaceutical composition targeting a TGFB1 gene.

In some embodiments, the transforming growth factor beta receptor gene is an TGFB1 gene. In some embodiments, the TGFB1 gene is a human TGFB1 gene and the guide RNA comprises a spacer sequence selected from SEQ ID NOs: 1-198.

2. Treatment of Fibrosis and/or Scarring by Targeting a Transforming Growth Factor Beta Gene

In one aspect, methods and pharmaceutical compositions are provided for treating fibrosis and/or scarring using a CRISPR pharmaceutical. In one aspect, methods and pharmaceutical compositions are provided for treating fibrosis and/or scarring using a CRISPR pharmaceutical composition that targets a Transforming Growth Factor Beta gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta gene. In some embodiments, the fibrosis and/or scarring is due to knee arthrofibrosis, intra-articular fibrous nodules, or epidural fibrosis and is treated with a CRISPR pharmaceutical composition targeting a TGFB1 gene. In some embodiments, a CRISPR pharmaceutical composition as described herein is a post-surgical treatment to prevent or reduce fibrosis and/or scarring. For example, in some embodiments, the surgical procedure is selected from ligament reconstruction, anterior cruciate ligament (ACL) reconstruction, autograft ACL reconstruction, allograft ACL reconstruction, tendon repair, fracture repair, total knee arthroplasty (TKA), microdiscectomy after which fibrosis and/or scarring is treated with a CRISPR pharmaceutical composition targeting a TGFB1 gene. In some embodiments, the fibrosis and/or scarring is the result of a condition, which may be induced or exacerbated by a surgical procedure.

In some embodiments, the transforming growth factor beta receptor gene is an TGFB1 gene. In some embodiments, the TGFB1 gene is a human TGFB1 gene and the guide RNA comprises a spacer sequence selected from SEQ ID NOs: 1-198.

### B. TGFBR1

In some embodiments, the pharmaceutical composition comprising a CRISPR gene-editing system targets TGFBR1. In some embodiments, the CRISPR gene-editing system targeting TGFBR1 is delivered to a mammalian cell via viral vector. In some embodiments, the CRISPR gene-editing system targeting TGFBR1 is delivered to a mammalian cell via an AAV vector. In some embodiments, the CRISPR gene-editing system targeting TGFBR1 is delivered to a mammalian cell via a lentiviral vector. In some embodiments, the CRISPR gene-editing system targeting TGFBR1 is delivered to a mammalian cell via a lipid nanoparticle. In some embodiments, the CRISPR gene-editing system targeting TGFBR1 is delivered to a mammalian cell via a virus-like particle. In some embodiments, the CRISPR gene-editing system targeting TGFBR1 is delivered to a mammalian cell via a liposome. In some embodiments, the CRISPR gene-editing system targeting TGFBR1 is delivered to a mammalian cell via a lipid nanocrystal. In some embodiments, the CRISPR gene-editing system targeting TGFBR1 is delivered to a mammalian cell via a polymeric nanoparticle. In some embodiments, the CRISPR gene-editing system targeting TGFBR1 is delivered to a mammalian cell via an exosome.

In various embodiments, the pharmaceutical composition comprising a CRISPR gene-editing system that targets the TGFBR1 gene is used in a method of treating a mammal in need thereof. In some embodiments, the method treats fibrosis and/or scarring. In some embodiments, the method treats one or more musculoskeletal diseases, conditions, and illnesses, including, but not limited to, Loeys-Dietz Syndrome, osteoarthritis, tendon injury, Marfan syndrome, aortic aneurysm (e.g., familial thoracic 3 aortic aneurysm), craniofacial abnormalities, and combinations thereof. In other embodiments, the method treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

### 1. Treatment of Musculoskeletal Disorders by Targeting a Transforming Growth Factor Beta Receptor 1 Gene

In one aspect, methods and pharmaceutical compositions are provided for treating a musculoskeletal disorder using a CRISPR pharmaceutical. In one aspect, methods and pharmaceutical compositions are provided for treating a musculoskeletal disorder using a CRISPR pharmaceutical composition that targets a Transforming Growth Factor Beta Receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta Receptor 1 gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta Receptor gene. In some embodiments, the mRNA encoding the RNA-guided nuclease and the at least one gRNA are packaged in a lipid nanoparticle (LNP). In some embodiments, the musculoskeletal disorder is Loeys-Dietz Syndrome, Osteoarthrosis, Marfan Syndrome, Aortic aneurysm (familial thoracic 3), or Craniofacial abnormalities is treated with a CRISPR pharmaceutical composition targeting a TGFBR1 or TGFBR2 gene.

In some embodiments, the transforming growth factor beta receptor gene is an TGFBR1 gene. In some embodiments, the TGFBR1 gene is a human TGFBR1 gene and the guide RNA comprises a spacer sequence selected from SEQ ID NOs: 199-320. In some embodiments, editing of the TGFBR1 gene results in a knockout of the TGFBR1 gene. In some such embodiments, the TGFBR1 gene is a human TGFBR1 gene and the guide RNA comprises a spacer sequence selected from SEQ ID NOs: 199-228. In some embodiments, editing of the TGFBR1 gene results in a gene that expresses a membrane-bound receptor decoy protein. In some such embodiments, the TGFBR1 gene is a human TGFBR1 gene and the guide RNA comprises a spacer sequence selected from SEQ ID NOs: 229-308 and SEQ ID NOs: 317-318. In some embodiments, editing of the TGFBR1 gene results in a gene that expresses a soluble receptor decoy protein. In some such embodiments, the TGFBR1 gene is a human TGFBR1 gene and the guide RNA comprises a spacer sequence selected from SEQ ID NOs: 309-318.

### 2. Treatment of Fibrosis by Targeting a Transforming Growth Factor Beta Receptor 1 Gene

In one aspect, methods and pharmaceutical compositions are provided for treating fibrosis and/or scarring using a CRISPR pharmaceutical. In one aspect, methods and pharmaceutical compositions are provided for treating fibrosis and/or scarring using a CRISPR pharmaceutical composition that targets a Transforming Growth Factor Beta Receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta Receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta Receptor gene. In some embodiments, the fibrosis and/or scarring is due to knee arthrofibrosis, intra-articular fibrous nodules, or epidural fibrosis and is treated with a CRISPR pharmaceutical composition targeting a TGFBR1 or TGFBR2 gene. In some embodiments, a CRISPR pharmaceutical composition as described herein is a post-surgical treatment to prevent or reduce fibrosis and/or scarring. For example, in some embodiments, the surgical procedure is selected from ligament reconstruction, anterior cruciate ligament (ACL) reconstruction, autograft ACL reconstruction, allograft ACL reconstruction, tendon repair, fracture repair, total knee arthroplasty (TKA), microdiscectomy after which fibrosis and/or scarring is treated with a CRISPR pharmaceutical composition targeting a TGFBR1 or TGFBR2 gene. In some embodiments, the fibrosis and/or scarring is the result of a condition, which may be induced or exacerbated by a surgical procedure.

In some embodiments, the transforming growth factor beta receptor gene is a TGFBR1 gene. In some embodiments, the TGFBR1 gene is a human TGFBR1 gene and the guide RNA comprises a spacer sequence selected from SEQ ID NOs: 199-320. In some embodiments, editing of the TGFBR1 gene results in a knockout of the TGFBR1 gene. In some such embodiments, the TGFBR1 gene is a human TGFBR1 gene and the guide RNA comprises a spacer sequence selected from SEQ ID NOs: 199-228. In some embodiments, editing of the TGFBR1 gene results in a gene that expresses a membrane-bound receptor decoy protein. In some such embodiments, the TGFBR1 gene is a human TGFBR1 gene and the guide RNA comprises a spacer sequence selected from SEQ ID NOs: 229-308 and SEQ ID NOs: 317-318. In some embodiments, editing of the TGFBR1 gene results in a gene that expresses a soluble receptor decoy protein. In some such embodiments, the TGFBR1 gene is a human TGFBR1 gene and the guide RNA comprises a spacer sequence selected from SEQ ID NOs: 309-318.

### C. TGFBR2

In some embodiments, the pharmaceutical composition comprising a CRISPR gene-editing system targets TGFBR2. In some embodiments, the CRISPR gene-editing system targeting TGFBR2 is delivered to a mammalian cell via viral vector. In some embodiments, the CRISPR gene-editing system targeting TGFBR2 is delivered to a mammalian cell via an AAV vector. In some embodiments, the CRISPR gene-editing system targeting TGFBR2 is delivered to a mammalian cell via a lentiviral vector. In some embodiments, the CRISPR gene-editing system targeting TGFBR2 is delivered to a mammalian cell via a lipid nanoparticle. In some embodiments, the CRISPR gene-editing system targeting TGFBR2 is delivered to a mammalian cell via a virus-like particle. In some embodiments, the CRISPR gene-editing system targeting TGFBR2 is delivered to a mammalian cell via a liposome. In some embodiments, the CRISPR gene-editing system targeting TGFBR2 is delivered to a mammalian cell via a lipid nanocrystal. In some embodiments, the CRISPR gene-editing system targeting TGFBR2 is delivered to a mammalian cell via a polymeric nanoparticle. In some embodiments, the CRISPR gene-editing system targeting TGFBR2 is delivered to a mammalian cell via an exosome.

In various embodiments, the pharmaceutical composition comprising a CRISPR gene-editing system that targets the TGFBR2 gene is used in a method of treating a mammal in need thereof. In some embodiments, the method treats fibrosis and/or scarring. In some embodiments, the method treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, the method treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

### 1. Treatment of Musculoskeletal Disorders by Targeting a Transforming Growth Factor Beta Receptor 2 Gene

In one aspect, methods and pharmaceutical compositions are provided for treating a musculoskeletal disorder using a CRISPR pharmaceutical. In one aspect, methods and pharmaceutical compositions are provided for treating a musculoskeletal disorder using a CRISPR pharmaceutical composition that targets a Transforming Growth Factor Beta Receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta Receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta Receptor gene. In some embodiments, the mRNA encoding the RNA-guided nuclease and the at least one gRNA are packaged in a lipid nanoparticle (LNP). In some embodiments, the musculoskeletal disorder is Loeys-Dietz Syndrome, Osteoarthrosis, Marfan Syndrome, Aortic aneurysm (familial thoracic 3), or Craniofacial abnormalities is treated with a CRISPR pharmaceutical composition targeting a TGFBR1 or TGFBR2 gene.

In some embodiments, the transforming growth factor beta receptor gene is an TGFBR2 gene. In some embodiments, the TGFBR2 gene is a human TGFBR2 gene and the guide RNA comprises a spacer sequence selected from SEQ ID NOs: 321-519. In some embodiments, editing of the TGFBR2 gene results in a knockout of the TGFBR2 gene. In some such embodiments, the TGFBR2 gene is a human TGFBR2 gene and the guide RNA comprises a spacer sequence selected from SEQ ID NOs: 321-354. In some embodiments, editing of the TGFBR2 gene results in a gene that expresses a membrane-bound receptor decoy protein. In some such embodiments, the TGFBR2 gene is a human TGFBR2 gene and the guide RNA comprises a spacer sequence selected from SEQ ID NOs: 355-498 and SEQ ID NOs: 503-508. In some embodiments, editing of the TGFBR2 gene results in a gene that expresses a soluble receptor decoy protein. In some such embodiments, the TGFBR2 gene is a human TGFBR2 gene and the guide RNA comprises a spacer sequence selected from SEQ ID NOs: 499-508.

2. Treatment of Fibrosis and/or Scarring by Targeting a Transforming Growth Factor Beta Receptor 2 Gene

In one aspect, methods and pharmaceutical compositions are provided for treating fibrosis and/or scarring using a CRISPR pharmaceutical. In one aspect, methods and pharmaceutical compositions are provided for treating fibrosis and/or scarring using a CRISPR pharmaceutical composition that targets a Transforming Growth Factor Beta Receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta Receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta Receptor gene. In some embodiments, the fibrosis and/or scarring is due to knee arthrofibrosis, intra-articular fibrous nodules, or epidural fibrosis and is treated with a CRISPR pharmaceutical composition targeting a TGFBR1 or TGFBR2 gene. In some embodiments, a CRISPR pharmaceutical composition as described herein is a post-surgical treatment to prevent or reduce fibrosis and/or scarring. For example, in some embodiments, the surgical procedure is selected from ligament reconstruction, anterior cruciate ligament (ACL) reconstruction, autograft ACL reconstruction, allograft ACL reconstruction, tendon repair, fracture repair, total knee arthroplasty (TKA), microdiscectomy after which fibrosis and/or scarring is treated with a CRISPR pharmaceutical composition targeting a TGFBR1 or TGFBR2 gene. In some embodiments, the fibrosis and/or scarring is the result of a condition, which may be induced or exacerbated by a surgical procedure.

In some embodiments, the transforming growth factor beta receptor gene is an TGFBR2 gene. In some embodiments, the TGFBR2 gene is a human TGFBR2 gene and the guide RNA comprises a spacer sequence selected from SEQ ID NOs: 321-519. In some embodiments, editing of the TGFBR2 gene results in a knockout of the TGFBR2 gene. In some such embodiments, the TGFBR2 gene is a human TGFBR2 gene and the guide RNA comprises a spacer sequence selected from SEQ ID NOs: 321-354. In some embodiments, editing of the TGFBR2 gene results in a gene that expresses a membrane-bound receptor decoy protein. In some such embodiments, the TGFBR2 gene is a human TGFBR2 gene and the guide RNA comprises a spacer sequence selected from SEQ ID NOs: 355-498 and SEQ ID NOs: 503-508. In some embodiments, editing of the TGFBR2 gene results in a gene that expresses a soluble receptor decoy protein. In some such embodiments, the TGFBR2 gene is a human TGFBR2 gene and the guide RNA comprises a spacer sequence selected from SEQ ID NOs: 499-508.

### VII. Administration Routes

The methods and compositions herein described encompass the use of pharmaceutical compositions comprising a CRISPR gene-editing system as an active ingredient.

Depending on the method/route of administration, pharmaceutical dosage forms come in several types. These include many kinds of liquid, solid, and semisolid dosage forms. Common pharmaceutical dosage forms include pill, tablet, or capsule, drink or syrup, and natural or herbal form such as plant or food of sorts, among many others. Notably, the route of administration (ROA) for drug delivery is dependent on the dosage form of the substance in question. A liquid pharmaceutical dosage form is the liquid form of a dose of a chemical compound used as a drug or medication intended for administration or consumption.

As described below, a composition of the present disclosure can be delivered to a subject subcutaneously (e.g., intra-articular or intradiscal injection), dermally (e.g., transdermally via patch), and/or via implant. Exemplary pharmaceutical dosage forms include, e.g., pills, osmotic delivery systems, elixirs, emulsions, hydrogels, suspensions, syrups, capsules, tablets, orally dissolving tablets (ODTs), gel capsules, thin films, adhesive topical patches, lollipops, lozenges, chewing gum, dry powder inhalers (DPIs), vaporizers, nebulizers, metered dose inhalers (MDIs), ointments, transdermal patches, intradermal implant.

As used herein, “dermal delivery” or “dermal administration” can refer to a route of administration wherein the pharmaceutical dosage form is taken to, or through, the dermis (i.e., layer of skin between the epidermis (with which it makes up the cutis) and subcutaneous tissues). “Subcutaneous delivery” can refer to a route of administration wherein the pharmaceutical dosage form is to or beneath the subcutaneous tissue layer.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Pharmaceutical compositions suitable for injectable use can also include cryopreservatives, which improve the stability of the formulation, particularly when frozen. For further discussion on conditions useful for storage of LNPs, see Kim B, et al., “Optimization of storage conditions for lipid nanoparticle-formulated self-replicating RNA vaccines,” J. Control Release, 353:241-53 (2023), the disclosure of which is incorporated herein by reference in its entirety for all purposes.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88 (2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy) propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethylene glycol-coated liposomes, hyaluronic acid and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006 December; 79 (3-4): 141-152.

The pharmaceutical compositions described herein may be included in a container, kit, pack, or dispenser together with instructions for administration.

### A. Systemic Administration

In some embodiments, a pharmaceutical composition comprising a CRISPR gene-editing system is administered systemically to a mammal in need thereof. In some embodiments, the composition is formulated for intravenous injection. In some embodiments, the composition is formulated for oral administration. In some embodiments, the composition is formulated for parenteral administration.

### B. Local Administration

In some embodiments, a pharmaceutical composition comprising a CRISPR gene-editing system is administered locally to a mammal in need thereof. In some embodiments, the local administration is an intra-articular injection. In some embodiments, the composition is formulated for intradiscal injection. In some embodiments, the composition is formulated for epidural injection. In some embodiments, the composition is formulated for peridiscal injection. In some embodiments, the composition is formulated for perivertebral injection. In some embodiments, composition is formulated for administration to the facet joints of the spine.

In some embodiments, a pharmaceutical composition comprising a CRISPR gene-editing system is administered locally to a mammal in need thereof during a surgical procedure. In some embodiments, a pharmaceutical composition comprising a CRISPR gene-editing system is administered locally to a mammal in need thereof 1, 2, 3, 4, 5, 6, 7, 8, 9, 10, 20, or 30 days after a surgical procedure.

### 1. Post-Musculoskeletal Trauma Administration

In some embodiments, compositions and methods disclosed herein are administered to a subject post-musculoskeletal trauma. In some embodiments, the musculoskeletal trauma is the result of a surgical procedure (i.e., a surgical incision). In some embodiments, the musculoskeletal trauma is a wound.

In some embodiments, compositions containing an RNA-guided nuclease and sgRNA are applied to a wound or incision. In some embodiments the composition is a spray. In some embodiments, the composition is a patch. In further embodiments, the patch is a bio-absorbable patch. In some embodiments, the composition is a mesh. In some embodiments, the composition is a hydrogel.

Additional exemplary compositions within which an RNA-guided nuclease and sgRNA are applied to a wound or incision include the following (each of which are hereby incorporated by reference in their entireties for all purposes): U.S. Pat. Nos. 8,574,627, 9,204,953, 9,272,073, 9,592,324, 9,597,426, 9,993,298, 10471181, 10765423, 10835235, 11338062, 11426156; and US patent applications US20080039877A1, US20200345366A1, US20200069478A1.

In some embodiments, the disclosure provides apharmaceutical composition for treating or preventing a disorder having a symptom caused, at least in part, by intercellular signaling mediated through the transforming growth factor beta (TGFβ) signalling pathway, the composition comprising: (i) an RNA-guided nuclease or a nucleic acid encoding an RNA-guided nuclease; and (ii) at least one guide RNA or a nucleic acid encoding at least one guide RNA targeting a TGFB1 gene, TGFBR1 gene, TGFBR2 gene, or a combination thereof.

In some embodiments, the disorder is fibrosis.

In some embodiments, the fibrosis is postoperative fibrosis.

In some embodiments, the fibrosis is post-ligament reconstruction fibrosis.

In some embodiments, the fibrosis is post-anterior cruciate ligament (ACL) reconstruction fibrosis, post-autograft ACL reconstruction fibrosis, or post-allograft ACL reconstruction fibrosis, or post-tendon repair fibrosis.

In some embodiments, the fibrosis is knee arthrofibrosis.

In some embodiments, the fibrosis is due to intra-articular fibrous nodules.

In some embodiments, the fibrosis is post joint arthroplasty.

In some embodiments, the fibrosis is post-total knee arthroplasty (TKA).

In some embodiments, the fibrosis is related to tendon injury or surgery.

In some embodiments, the fibrosis is post-fracture repair fibrosis.

In some embodiments, the fibrosis is post microdiscectomy fibrosis.

In some embodiments, the fibrosis is post-microdiscectomy epidural fibrosis.

In some embodiments, the fibrosis is post-lumbar laminectomy fibrosis.

In some embodiments, the fibrosis is post-lumbar laminectomy epidural fibrosis.

In some embodiments, the fibrosis is post-anterior acromioplasty fibrosis.

In some embodiments, the fibrosis is post-subacromial decompression fibrosis.

In some embodiments, the fibrosis is post-anterior acromioplasty soft tissue fibrosis.

In some embodiments, the fibrosis is post-anterior acromioplasty joint contracture.

In some embodiments, the fibrosis is post-arthroscopy fibrosis.

In some embodiments, the fibrosis is post-arthroscopy arthrofibrosis.

In some embodiments, the fibrosis is fibrosis of the trabecular meshwork.

In some embodiments, fibrosis of the Trabecular Meshwork is post-glaucoma surgery fibrosis.

In some embodiments, the fibrosis is epidural fibrosis.

In some embodiments, the fibrosis is atrial fibrosis, cardiac fibrosis, myocardial fibrosis, and/or post-open heart surgery fibrosis.

In some embodiments, the fibrosis is post-liposuction fibrosis.

In some embodiments, the fibrosis is idiopathic pulmonary fibrosis (IPF).

In some embodiments, the fibrosis is fibrosis in a knee joint, a shoulder joint, or an elbow joint.

In some embodiments, the disorder includes fibrosis in a kidney tissue.

In some embodiments, the disorder is chronic kidney disease.

In some embodiments, the disorder includes fibrosis in a skin tissue.

In some embodiments, the disorder is scarring post wound healing, keloid disorder, nephrogenic systemic fibrosis, or scleroderma/systemic sclerosis.

In some embodiments, the disorder includes fibrosis in a lung tissue.

In some embodiments, the disorder is fibrothorax, pulmonary fibrosis, cystic fibrosis, idiopathic pulmonary fibrosis, radiation-induced lung injury, progressive massive fibrosis, or scleroderma/systemic sclerosis.

In some embodiments, the disorder includes fibrosis in a liver tissue.

In some embodiments, the disorder is cirrhosis or bridging fibrosis.

In some embodiments, the disorder includes fibrosis in a cardiac tissue.

In some embodiments, the fibrosis is interstitial fibrosis.

In some embodiments, the disorder is congestive heart failure or hypertension.

In some embodiments, the fibrosis is replacement fibrosis.

In some embodiments, the disorder is myocardial infarction.

In some embodiments, the disorder includes fibrosis in a brain tissue.

In some embodiments, the fibrosis is a glial scar.

In some embodiments, the disorder includes fibrosis in an intestinal tissue.

In some embodiments, the disorder is Crohn's disease.

In some embodiments, the disorder includes fibrosis in a hand or finger.

In some embodiments, the disorder is Dupuytren's contracture.

In some embodiments, the disorder includes fibrosis in a lymph tissue.

In some embodiments, the fibrosis is mediastinal fibrosis.

In some embodiments, the disorder includes fibrosis in a bone marrow tissue.

In some embodiments, the fibrosis is myelofibrosis.

In some embodiments, the disorder includes fibrosis in a penile tissue.

In some embodiments, the disorder is Peyronie's disease.

In some embodiments, the disorder includes fibrosis in a soft tissue of the retroperitoneum.

In some embodiments, the fibrosis is retroperitoneal fibrosis.

In some embodiments, the disorder includes fibrosis in a musculoskeletal tissue.

In some embodiments, the disorder is a musculoskeletal disorder.

In some embodiments, the musculoskeletal disorder is a genetic musculoskeletal disease.

In some embodiments, the musculoskeletal disorder is a muscular dystrophy.

In some embodiments, the musculoskeletal disorder is selected from Duchenne muscular dystrophy (DMD), myotonic dystrophy (DM), facioscapulohumeral muscular dystrophy (FSHD), and limb-girdle muscular dystrophy (LGMD).

In some embodiments, the musculoskeletal disorder is a type II collagenopathy.

In some embodiments, the musculoskeletal disorder is selected from achondrogenesis type II, hypochondrogenesis, Stickler Syndrome, and Czech Dysplasia.

In some embodiments, the musculoskeletal disorder is osteogenesis imperfecta (OI).

In some embodiments, the musculoskeletal disorder is an autoimmune musculoskeletal disease.

In some embodiments, the musculoskeletal disorder is an autoimmune musculoskeletal disease with polygenic susceptibility traits.

In some embodiments, the musculoskeletal disorder is a spondyloarthropathy (SA).

In some embodiments, the musculoskeletal disorder is ankylosing spondylitis.

In some embodiments, the musculoskeletal disorder is rheumatoid arthritis.

In some embodiments, the musculoskeletal disorder is autoimmune osteoarthritis.

In some embodiments, the musculoskeletal disorder is osteoarthritis (OA).

In some embodiments, the musculoskeletal disorder is Camurati-Engelmann disease.

In some embodiments, the musculoskeletal disorder is Sjogren's Syndrome.

In some embodiments, the musculoskeletal disorder is a mechanical musculoskeletal injury.

In some embodiments, the musculoskeletal disorder is selected from chronic muscle injury, torn tendon, post-traumatic osteoarthritis, and post-traumatic arthrofibrosis.

In some embodiments, the disorder is cancer. In some embodiments, the cancer is musculoskeletal cancer. In some embodiments, the cancer is one in which TGF-beta or TGFBR have been implicated.

In some embodiments, the at least one guide RNA or a nucleic acid encoding at least one guide RNA targets a mammalian TGFB1 gene.

In some embodiments, the mammalian TGFB1 gene is a canine, equine, or

feline TGFB1 gene.

In some embodiments, the mammalian TGFB1 gene is a human TGFB1 gene.

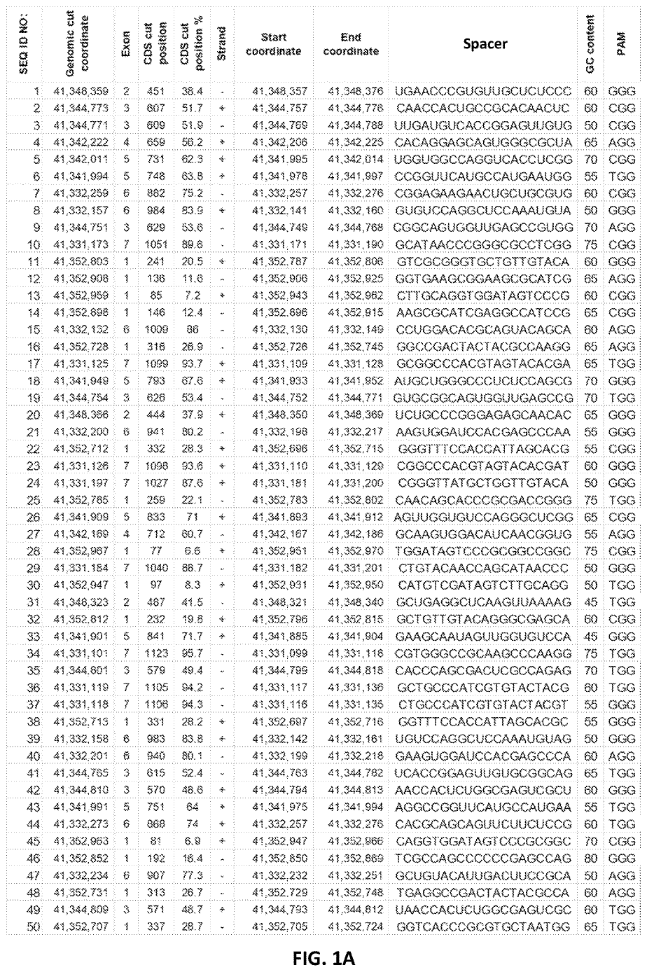

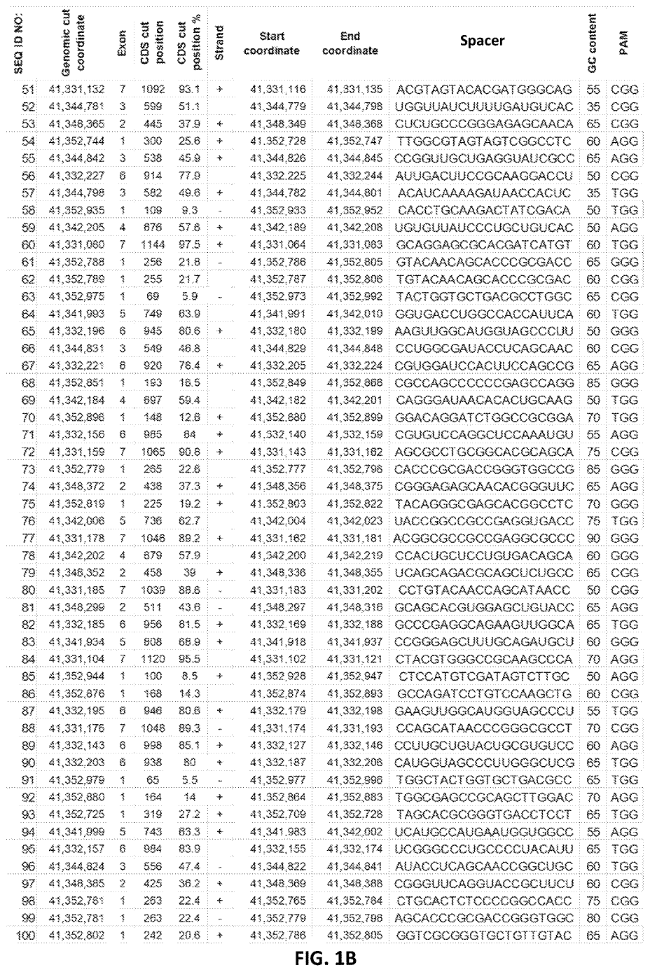

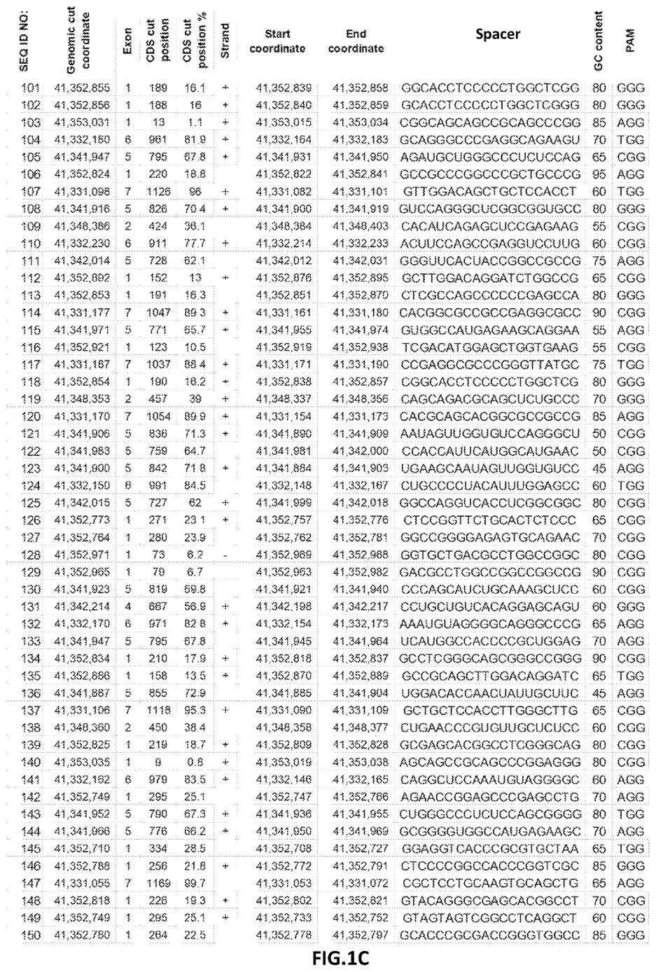

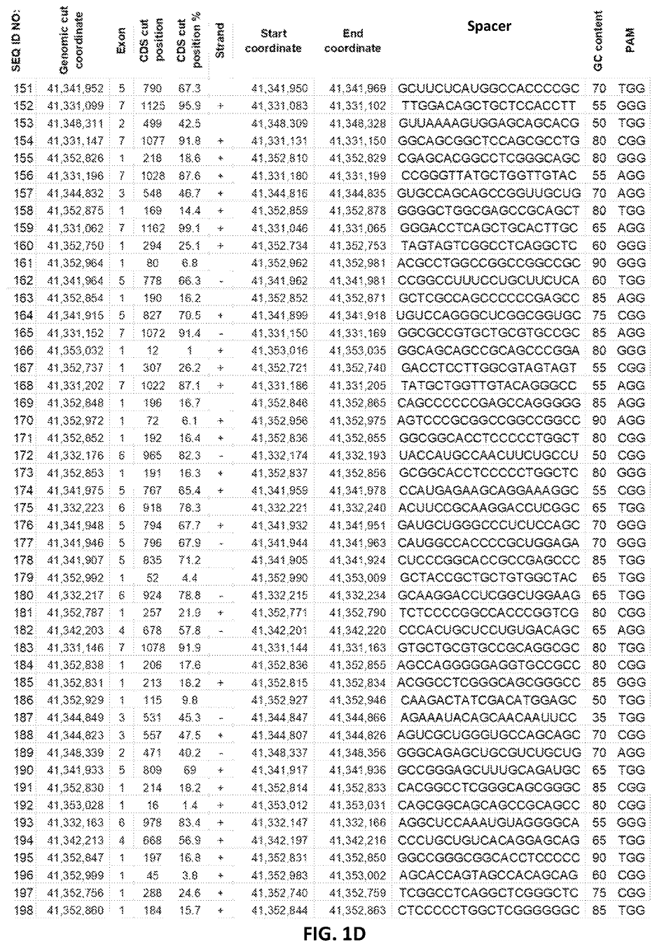

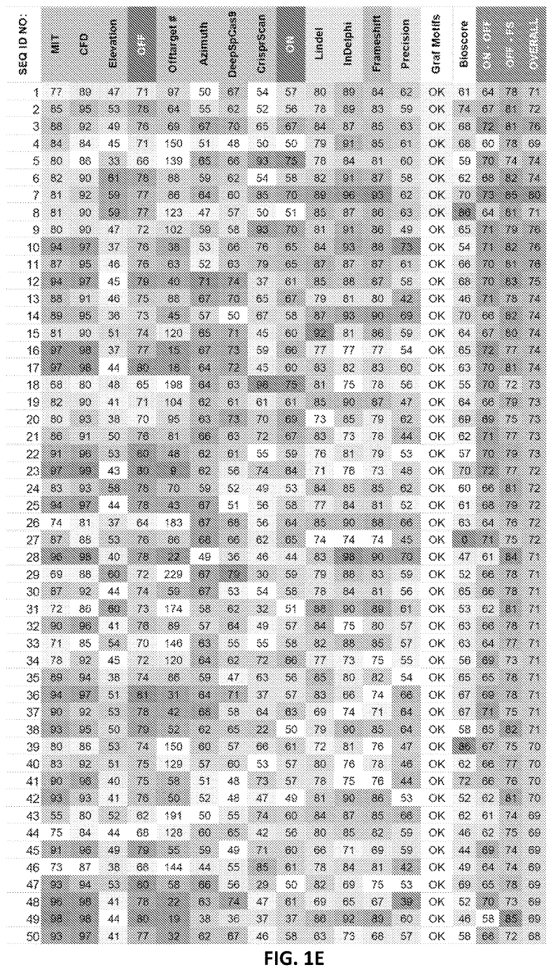

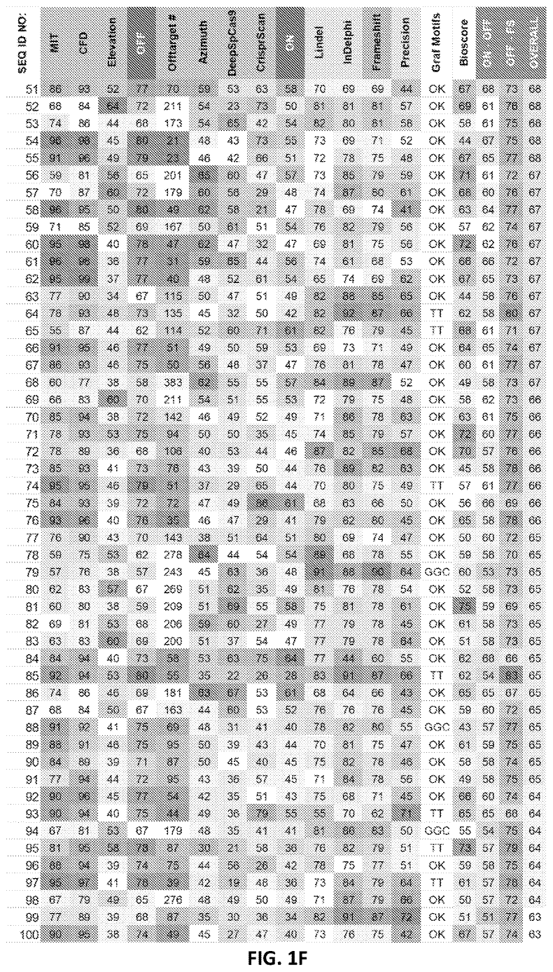

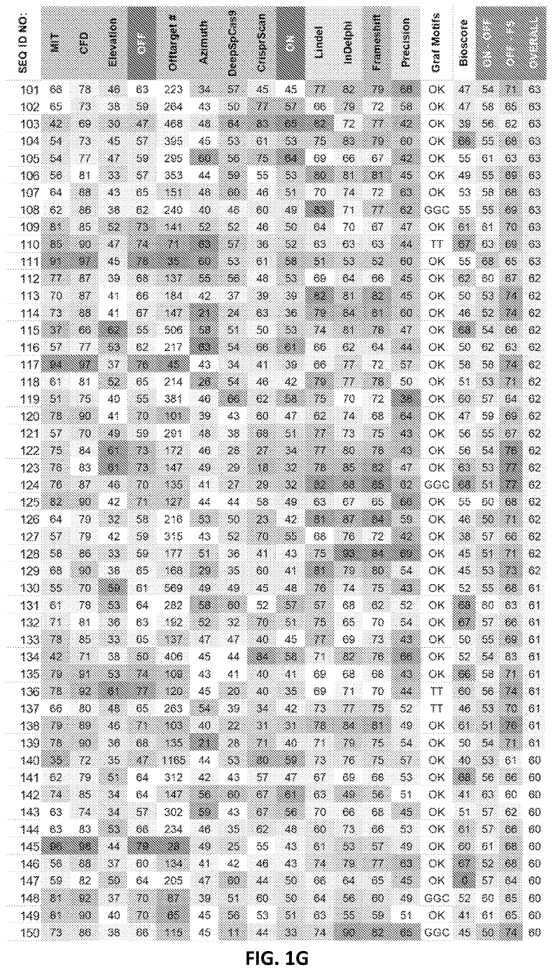

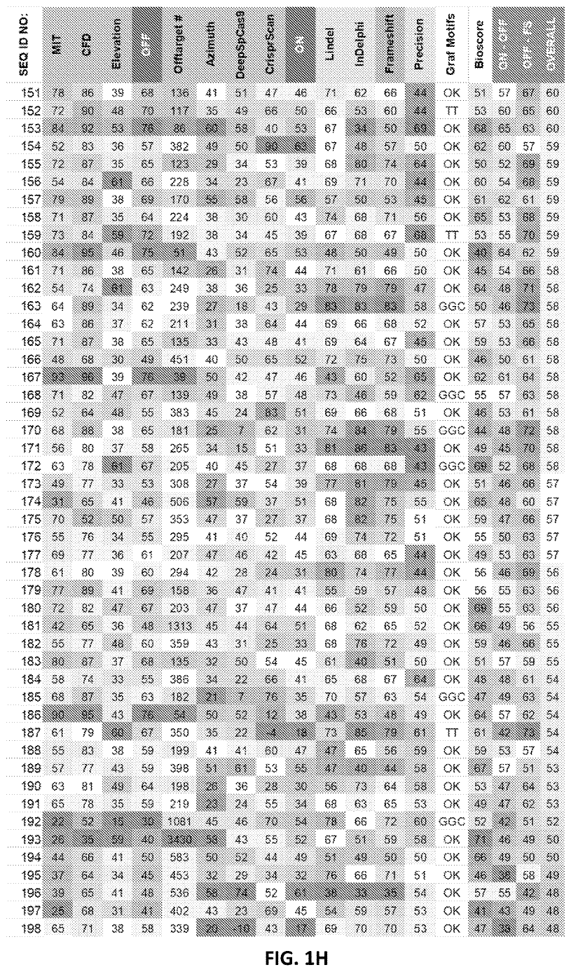

In some embodiments, the at least one guide RNA comprises a spacer sequence selected from the group consisting of those sequences shown in FIGS. 1A-1D (SEQ ID NOS: 1-198).

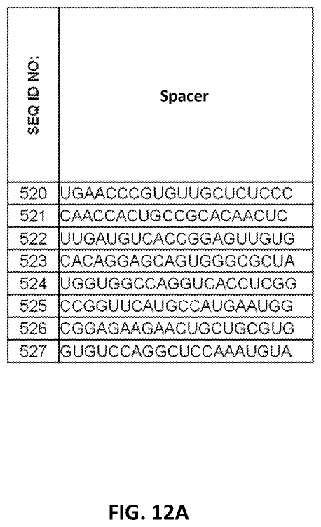

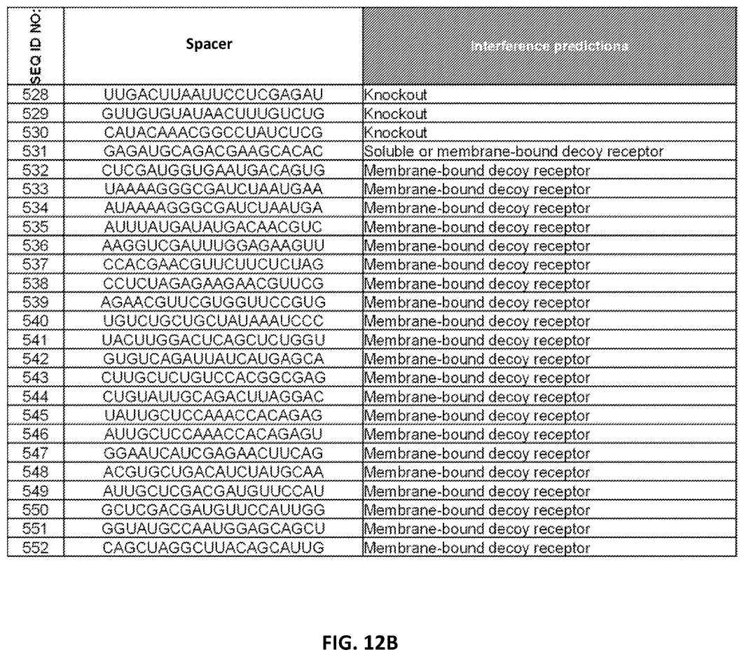

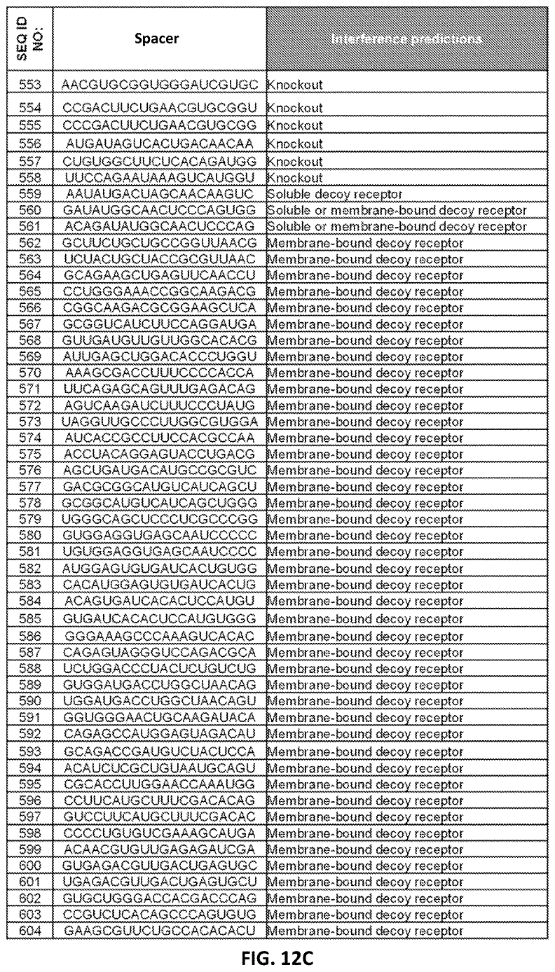

In some embodiments, the at least one guide RNA comprises a spacer sequence selected from the group consisting of those sequences shown in FIG. 12A (SEQ ID NOS: 520-527).

In some embodiments, the at least one guide RNA comprises a spacer sequence of OHTG03 (SEQ ID NO:522) or OHTG04 (SEQ ID NO:523).

In some embodiments, the at least one guide RNA or a nucleic acid encoding at least one guide RNA targets a mammalian TGFBR1 gene.

In some embodiments, the mammalian TGFBR1 gene is a canine, equine, or feline TGFBR1 gene.

In some embodiments, the mammalian TGFBR1 gene is a human TGFBR1 gene.

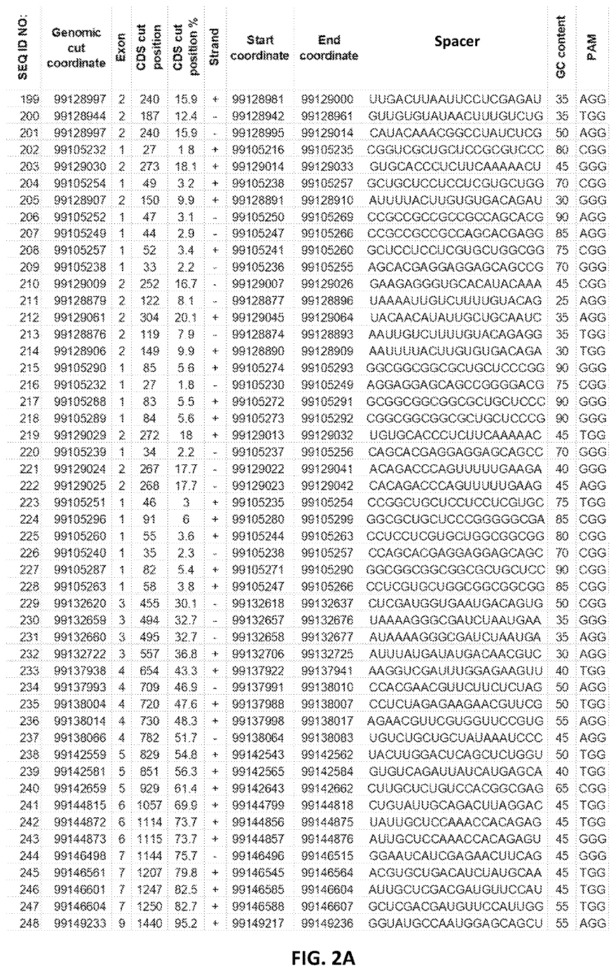

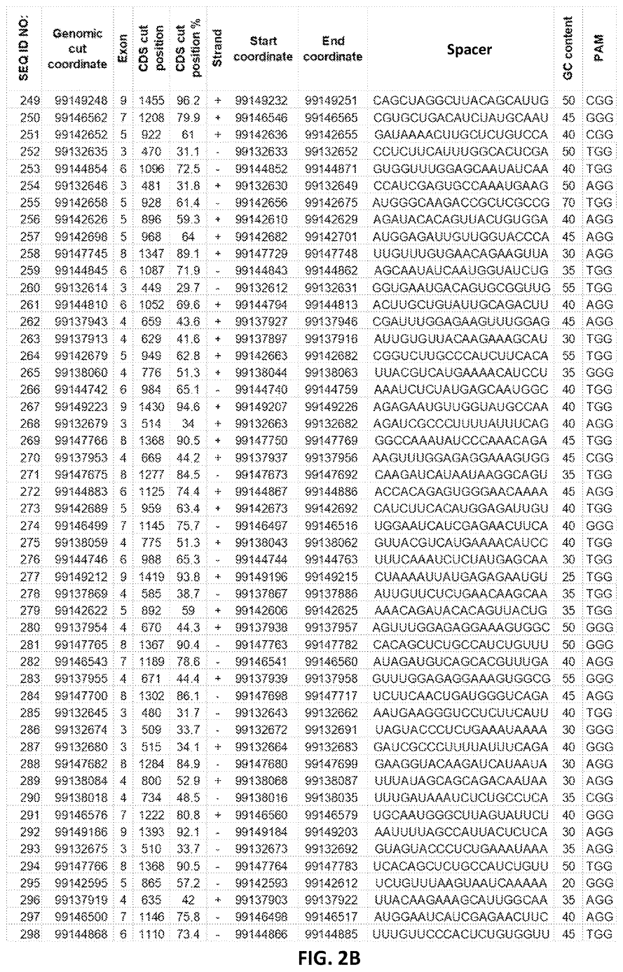

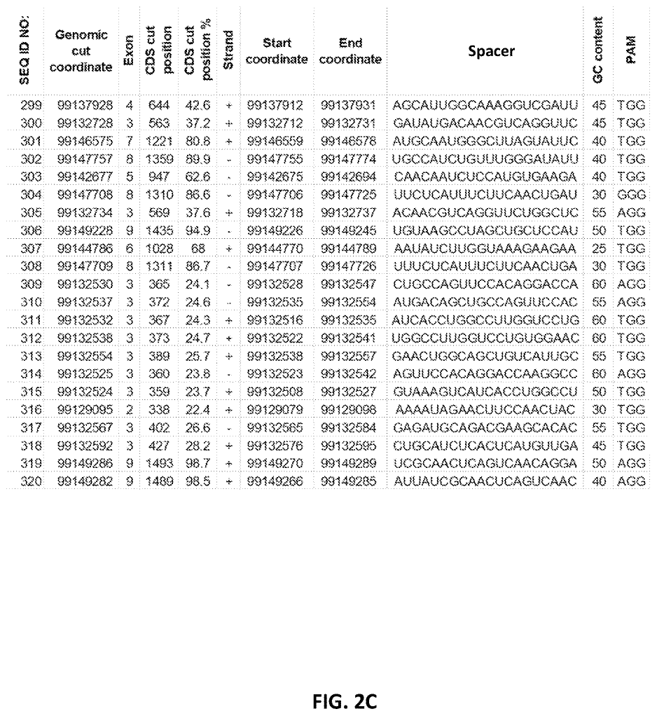

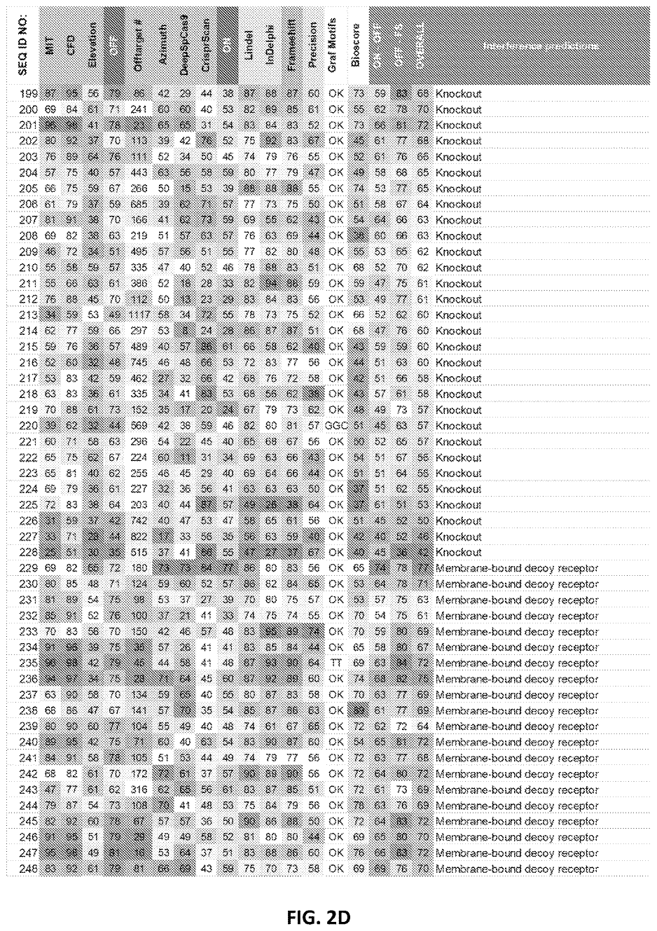

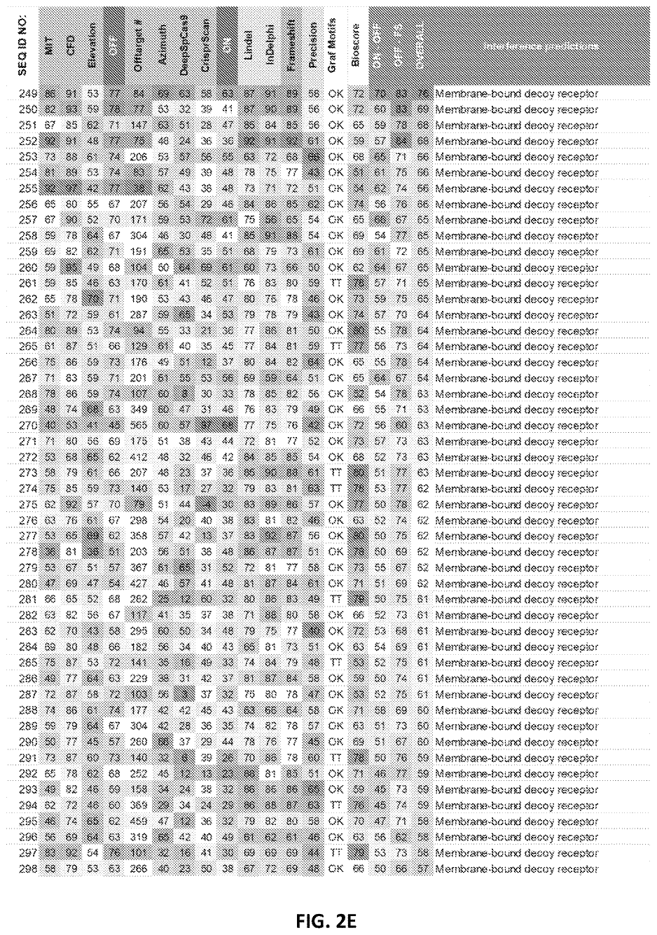

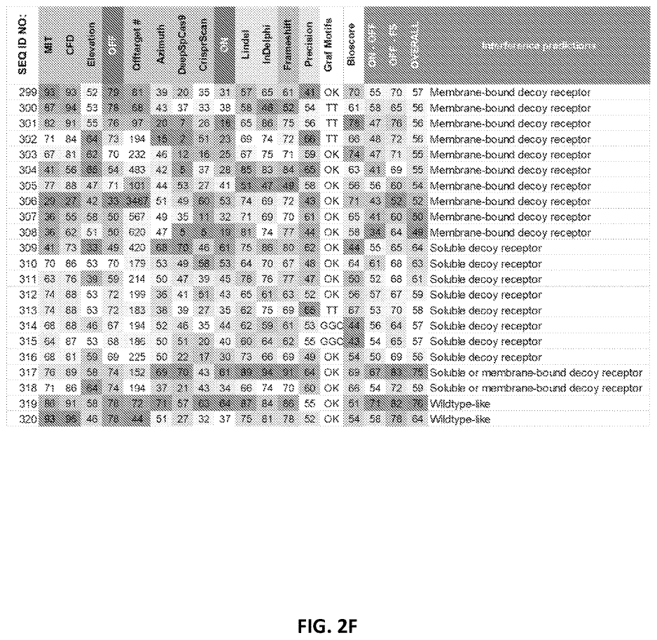

In some embodiments, the at least one guide RNA comprises a spacer sequence selected from the group consisting of those sequences shown in FIGS. 2A-2C (SEQ ID NOS: 199-320).

In some embodiments, the at least one guide RNA comprises a spacer sequence selected from the group consisting of those sequences shown in FIG. 12B (SEQ ID NOS: 528-552).

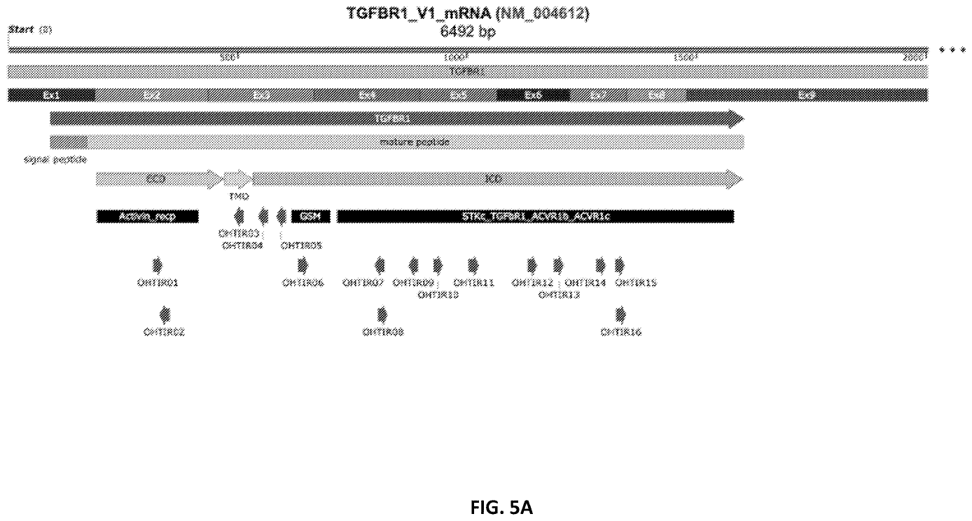

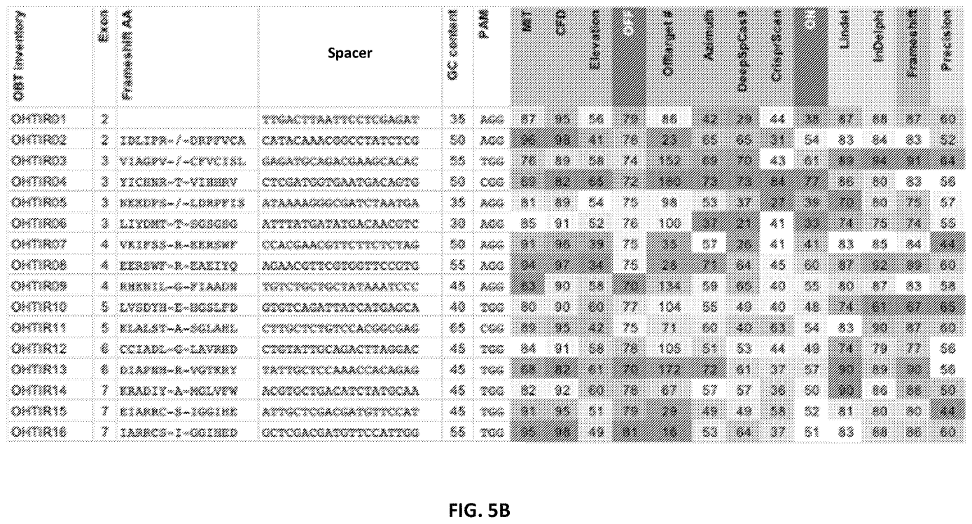

In some embodiments, the at least one guide RNA comprises a spacer sequence selected from the group consisting of those sequences shown in FIG. 5B.

In some embodiments, the at least one guide RNA comprises a spacer sequence of OHTIR04 (SEQ ID NO:532) or OHTIR08 (SEQ ID NO:539).

In some embodiments, the at least one guide RNA or a nucleic acid encoding at least one guide RNA targets a mammalian TGFBR2 gene.

In some embodiments, the mammalian TGFBR2 gene is a canine, equine, or feline TGFBR2 gene.

In some embodiments, the mammalian TGFBR2 gene is a human TGFBR2 gene.

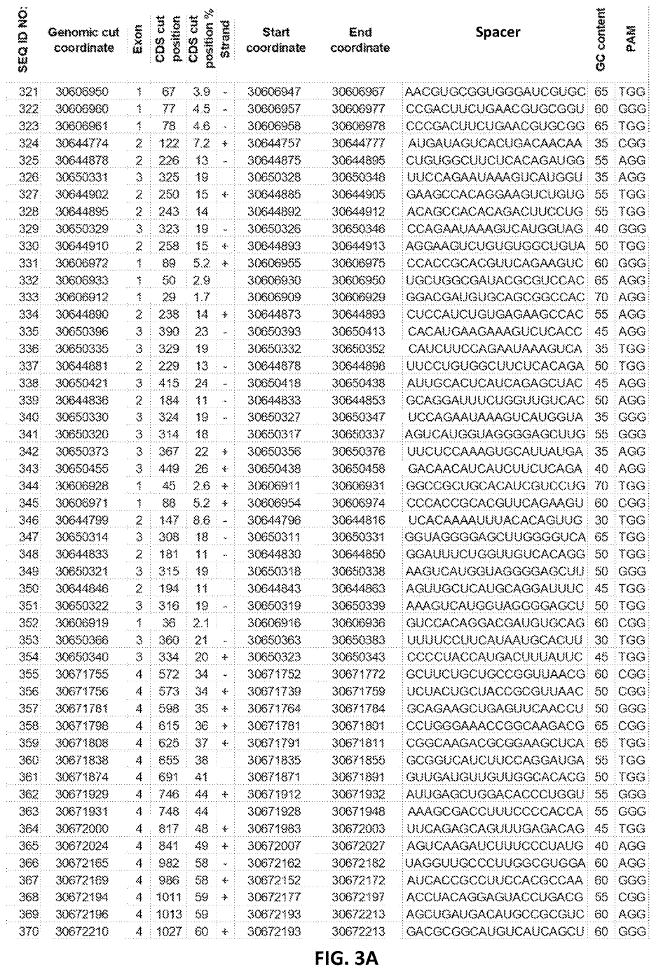

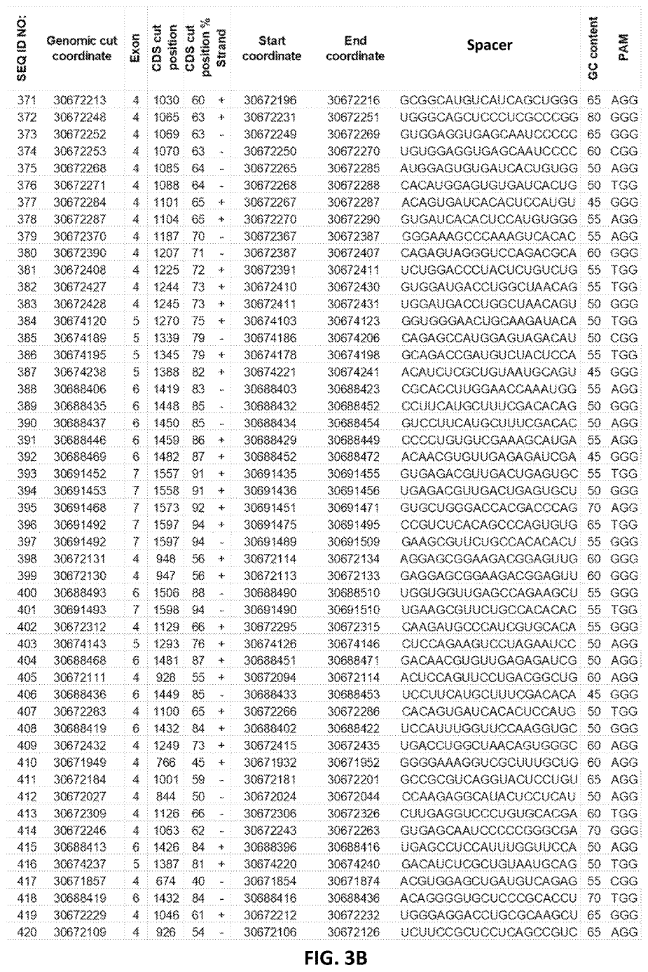

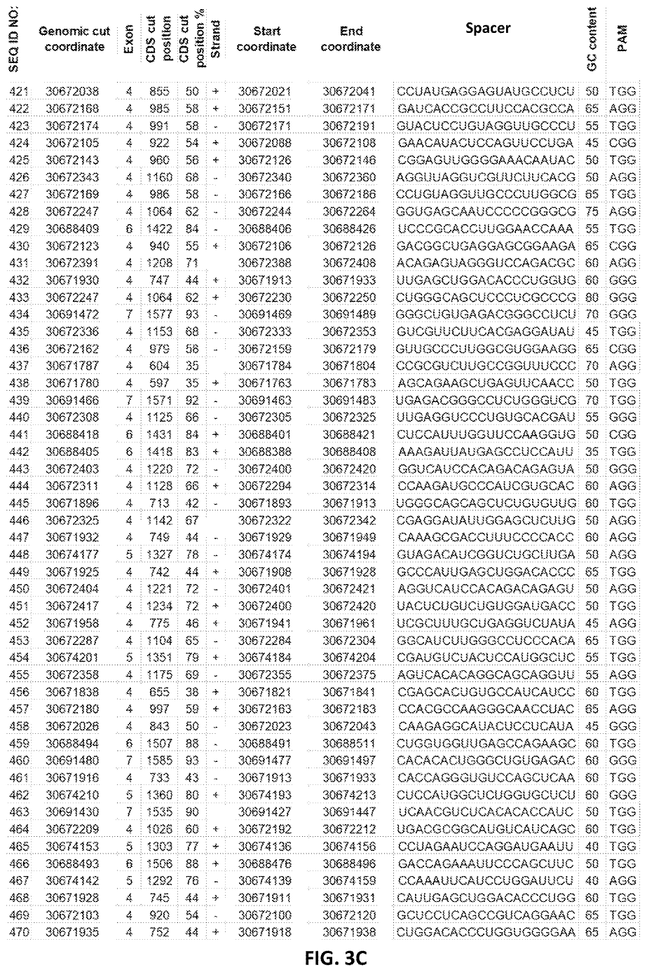

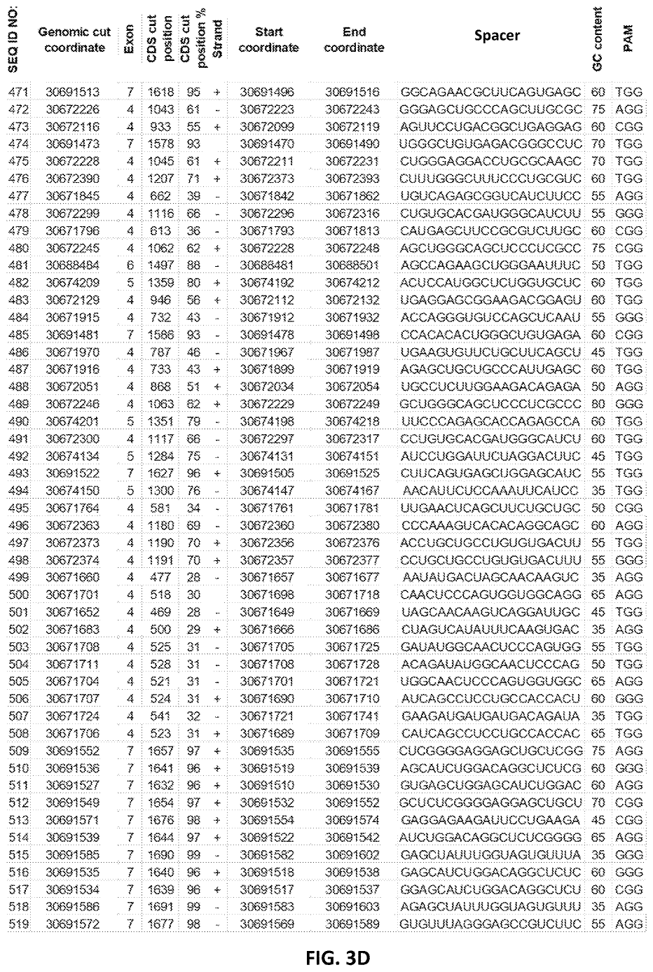

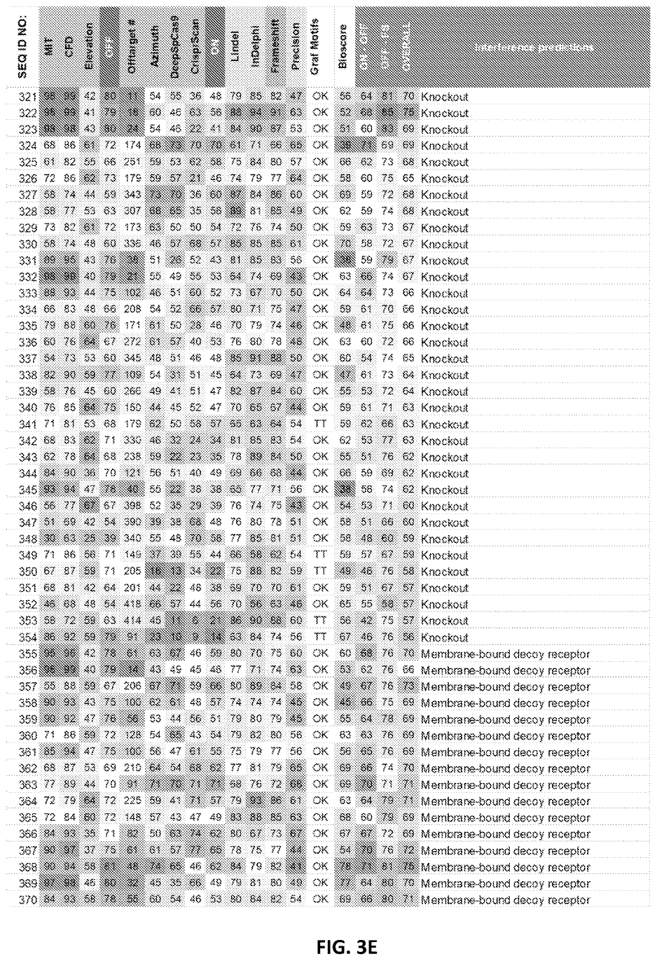

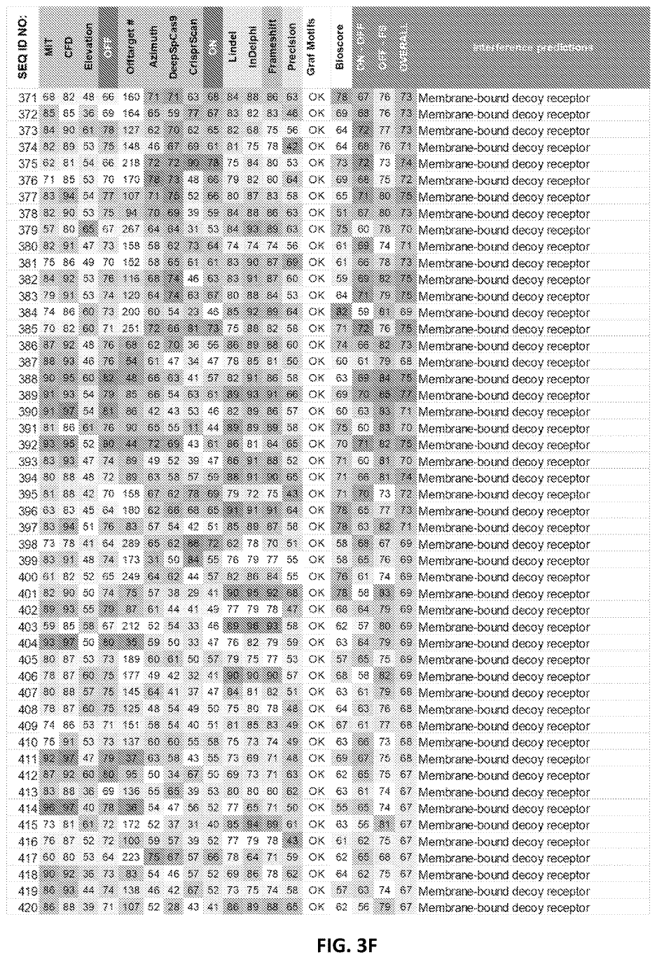

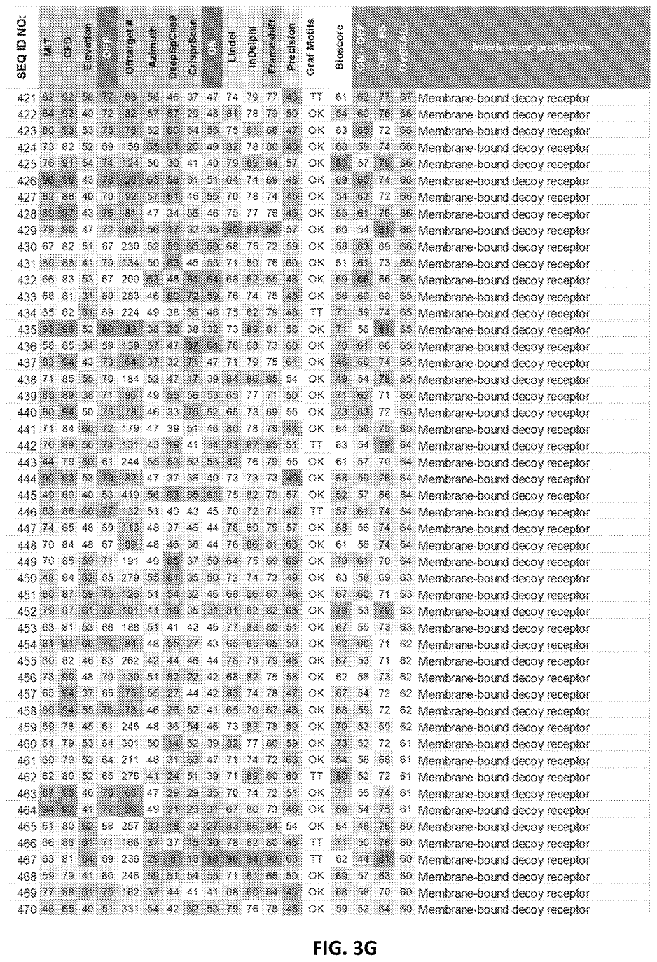

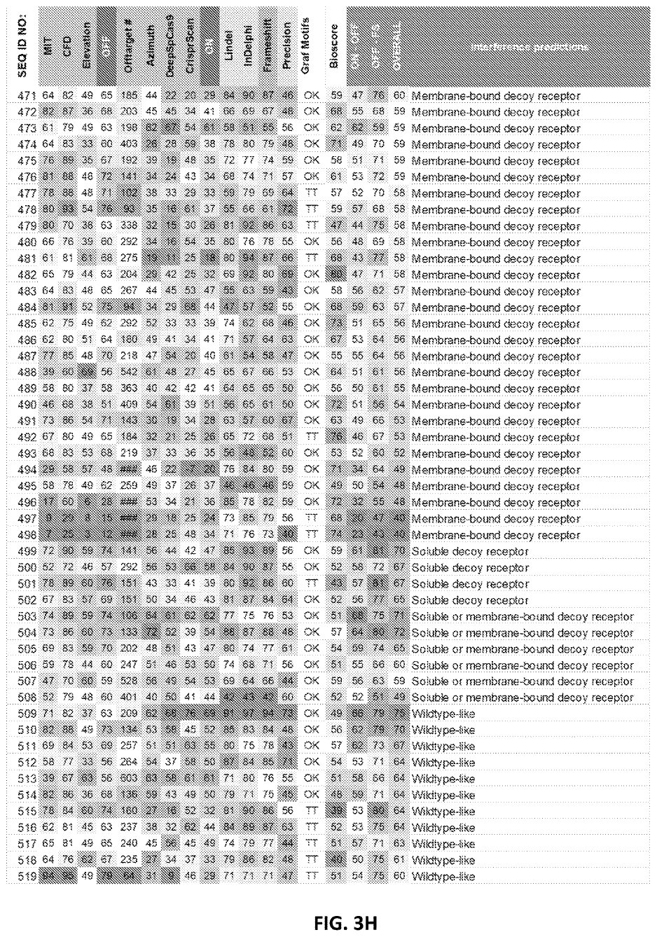

In some embodiments, the at least one guide RNA comprises a spacer sequence selected from the group consisting of those sequences shown in FIGS. 3A-3D (SEQ ID NOS: 321-519).

In some embodiments, the at least one guide RNA comprises a spacer sequence selected from the group consisting of those sequences shown in FIG. 12C (SEQ ID NOS: 553-604).

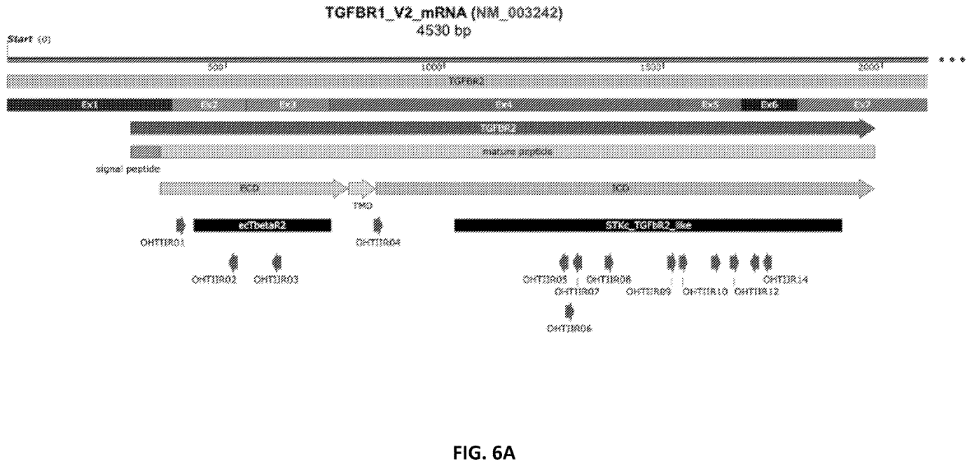

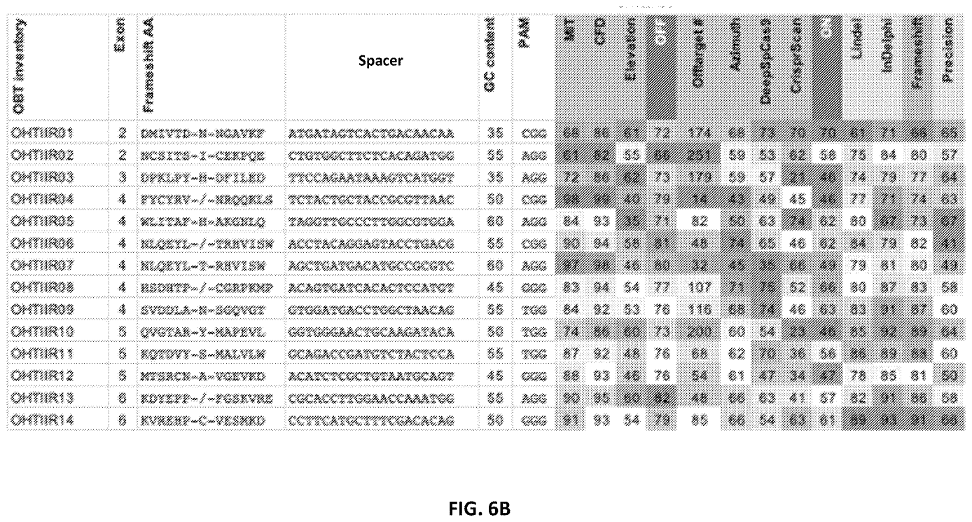

In some embodiments, the at least one guide RNA comprises a spacer sequence selected from the group consisting of those sequences shown in FIG. 6B.

In some embodiments, the at least one guide RNA comprises a spacer sequence of OHTIIR04 (SEQ ID NO:563).

In some embodiments, the RNA-guided nuclease or a nucleic acid encoding an RNA-guided nuclease is the RNA-guided nuclease.

In some embodiments, the RNA-guided nuclease or a nucleic acid encoding an RNA-guided nuclease is DNA encoding the RNA-guided nuclease.

In some embodiments, the RNA-guided nuclease or a nucleic acid encoding an RNA-guided nuclease is mRNA encoding the RNA-guided nuclease.

In some embodiments, the RNA-guided nuclease is a Cas protein.

In some embodiments, the Cas protein is a Cas9 protein.

In some embodiments, the Cas9 protein is an S. pyogenes Cas9 polypeptide.

In some embodiments, the Cas9 protein is selected from the group consisting of ESCas9, HFCas9, PECas9, and ARCas9.

In some embodiments, the at least one guide RNA or a nucleic acid encoding at least one guide RNA is the at least one guide RNA.

In some embodiments, the at least one guide RNA or a nucleic acid encoding at least one guide RNA is DNA encoding the at least one guide RNA.

In some embodiments, the pharmaceutical composition includes a nucleic acid encoding both the RNA-guided nuclease and the at least one guide RNA.

In some embodiments, the at least one guide RNA is a single guide RNA (sgRNA).

In some embodiments, the composition comprises one or more viral vectors collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one of more viral vectors comprise a recombinant virus selected from a retrovirus, an adenovirus, an adeno-associated virus, a lentivirus, and a herpes simplex virus-1.

In some embodiments, the one of more viral vectors comprise a recombinant adeno-associated virus (AAV).

In some embodiments, the recombinant AAV is of serotype 5 (AAV5).

In some embodiments, the recombinant AAV is of serotype 6 (AAV6).

In some embodiments, the composition comprises one or more lipid nanoparticles (LNP) collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one or more LNP comprises: a first plurality of LNP encapsulating the RNA-guided nuclease or a nucleic acid encoding an RNA-guided nuclease; and a second plurality of LNP encapsulating the at least one guide RNA or a nucleic acid encoding at least one guide RNA.

In some embodiments, the one or more LNP comprises a plurality of LNP encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

In some embodiments, the LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

In some embodiments, the LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

In some embodiments, the LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9, 11-dehydroergosterol, tomatidine, tomatine, a-tocopherol, and a mixture thereof.

In some embodiments, the composition comprises one or more liposomes collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one or more liposomes comprises: a first plurality of liposomes encapsulating the RNA-guided nuclease or a nucleic acid encoding an RNA-guided nuclease; and a second plurality of liposomes encapsulating the at least one guide RNA or a nucleic acid encoding at least one guide RNA.

In some embodiments, the one or more liposomes comprises a plurality of liposomes encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the composition comprises one or more virus-like particles collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one or more virus-like particles comprises:

In some embodiments, a first plurality of virus-like particles encapsulating the RNA-guided nuclease or a nucleic acid encoding an RNA-guided nuclease; and a second plurality of virus-like particles encapsulating the at least one guide RNA or a nucleic acid encoding at least one guide RNA.

In some embodiments, the one or more virus-like particles comprises a plurality of virus-like particles encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the composition is formulated for parenteral administration.

In some embodiments, the composition is formulated for intra-articular injection within a joint of the subject.

In some embodiments, the composition is formulated for intradiscal injection. In some embodiments, the composition is formulated for peridiscal injection.

In some embodiments, the composition is formulated for intravertebral injection.

In some embodiments, the disclosure provides a method for treating or preventing free oxygen radicals in a subject in need thereof by administering a therapeutically effective amount of a composition, wherein the composition comprises: (i) an RNA-guided nuclease or a nucleic acid encoding an RNA-guided nuclease; and (ii) at least one guide RNA or a nucleic acid encoding at least one guide RNA targeting a TGFB1 gene, TGFBR1 gene, TGFBR2 gene, or a combination thereof.

In some embodiments, the disclosure provides a method for treating or preventing a disorder having a symptom caused, at least in part, by intercellular signaling mediated through the transforming growth factor beta (TGFβ) signalling pathway in a subject in need thereof, comprising: administering a therapeutically effective amount of a composition, wherein the composition comprises: (i) an RNA-guided nuclease or a nucleic acid encoding an RNA-guided nuclease; and (ii) at least one guide RNA or a nucleic acid encoding at least one guide RNA targeting a TGFB1 gene, TGFBR1 gene, TGFBR2 gene, or a combination thereof.

In some embodiments, the disorder is fibrosis.

In some embodiments, the fibrosis is postoperative fibrosis.

In some embodiments, the fibrosis is post-ligament reconstruction fibrosis.

In some embodiments, the fibrosis is post-anterior cruciate ligament (ACL) reconstruction fibrosis, post-autograft ACL reconstruction fibrosis, or post-allograft ACL reconstruction fibrosis.

In some embodiments, the fibrosis is knee arthrofibrosis.

In some embodiments, the fibrosis is due to intra-articular fibrous nodules.

In some embodiments, the fibrosis is post joint arthroplasty.

In some embodiments, the fibrosis is post-total knee arthroplasty (TKA).

In some embodiments, the fibrosis is related to tendon injury or surgery.

In some embodiments, the fibrosis is post-fracture repair fibrosis.

In some embodiments, the fibrosis is post microdiscectomy fibrosis.

In some embodiments, the fibrosis is post-microdiscectomy epidural fibrosis.

In some embodiments, the fibrosis is post-lumbar laminectomy fibrosis.

In some embodiments, the fibrosis is post-lumbar laminectomy epidural fibrosis.

In some embodiments, the fibrosis is post-anterior acromioplasty fibrosis.

In some embodiments, the fibrosis is post-subacromial decompression fibrosis.

In some embodiments, the fibrosis is post-anterior acromioplasty soft tissue fibrosis.

In some embodiments, the fibrosis is post-anterior acromioplasty joint contracture.

In some embodiments, the fibrosis is post-arthroscopy fibrosis.

### VIII. Examples

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Bioinformatic Design of Guides for Disrupting TGF-Beta Signaling

A pipeline of in silico and in vitro screening was developed to identify candidate CRISPR-Cas guides for gene editing. The pipeline was applied to identify guides that effectively edit the human TGFB1 (hTGFB1), TGFBR1 (hTGFRB1), and TGFBR2 genes (hTGFRB2), thereby disrupting TGF-beta signalling in vivo to treat musculoskeletal fibrosis and/or scarring. The first step in the pipeline was to identify all possible spacer sequences of crRNA or sgRNA for a particular CRISPR-Cas protein in the coding portions of the hTGFB1, hTGFRB1, and hTGFRB2. Many algorithms for identifying such sequences are known in the art. Generally, these algorithms function by identifying a protospacer adjacent motif (PAM) sequence for the particular CRISPR-Cas protein and then locate the sequence spaced according to the requirements of the particular Cas protein, typically directly 5’ of the PAM site. For example, the S. pyogenes Cas9 (SpCas9) protein, which was used in this Example, recognizes a 5′-NGG-3′ PAM sequence. Thus, all sequences directly 5′ of an NGG trinucleotide are possible spacer sequences of crRNA or sgRNA. Spacer sequences identified in this fashion as shown in FIG. 27, along with biographical information about the spacer sequence.

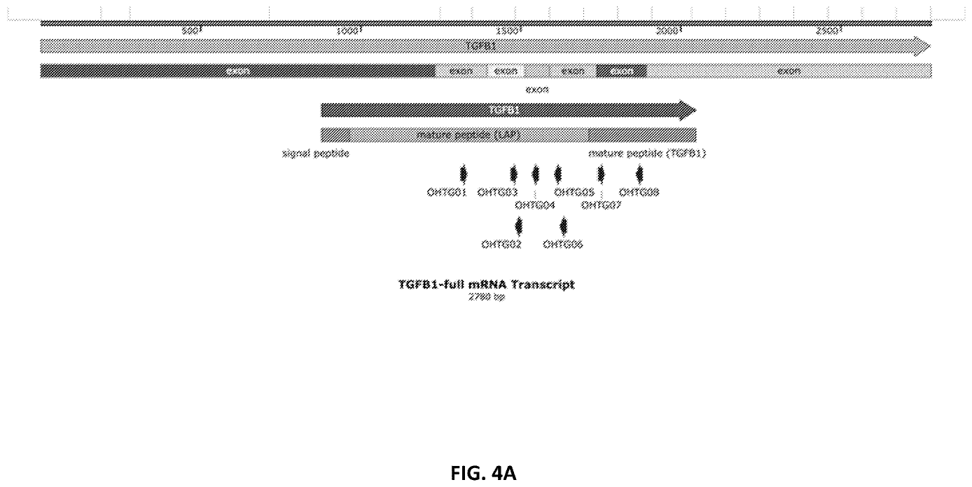

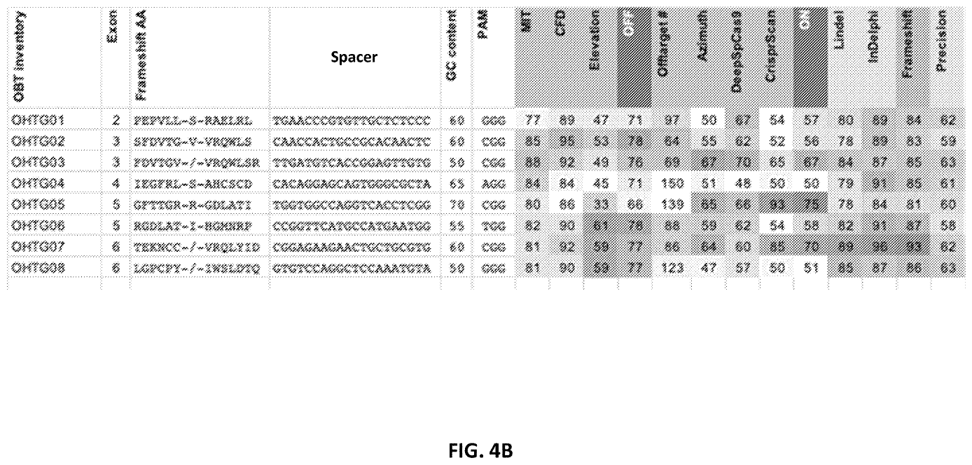

Next, each identified spacer sequence (see, FIGS. 1-3) was evaluated across three different metrics: possible off-target editing at locations in the genome other than the target gene, on-target editing efficiency, and the likelihood of editing causing frameshift mutations, using multiple algorithms for each metric, as illustrated in FIGS. 4B, 5B, and 6B. The basis of the combinatorial approach used lies in the assumption that every model has blind spots that may skew the fitness of a particular guide RNA. Weighting these scores to obtain a consensus score for each of these properties allows for much better prediction of sgRNA fitness.

Off-target editing effects were predicted by averaging scores generated by the MIT, CFD and Elevation (human only) model. The MIT algorithm, also known as Hsu-Zhang score. Hsu, P. D. et al. DNA targeting specificity of RNA-guided Cas9 nucleases. Nature Biotechnology 31, 827-832 (2013). This model is based on a positional penalty matrix (1×20) generated from 15 EMX1 sgRNA libraries with mismatches against target at every position. The CFD algorithm (Cutting Frequency Determination) is based on threat matrix (12×20) considering both position and mismatch type and PAM integrity (27,897 ‘CD33’ sgRNAs+10,618 negative control sgRNAs). Doench, J. G. et al. Optimized sgRNA design to maximize activity and minimize off-target effects of CRISPR-Cas9. Nature Biotechnology 34, 184-191 (2016). The Elevation score uses machine learning algorithms trained by genome-wide (GUIDE-Seq) and other aggregated off-target profiling data. Listgarten, J. et al. Prediction of off-target activities for the end-to-end design of CRISPR guide RNAs. Nature Biomedical Engineering 2, 38-47 (2018). The column labelled “OFF” shows the mean of the scores provided by the two or three models, respectively. The column labelled “OffTarget #” shows the number of potential off-targets with up to four mismatches as calculated by CRISPOR. See, Haeussler, M. et al. Evaluation of off-target and on-target scoring algorithms and integration into the guide RNA selection tool CRISPOR. Genome Biology 17, 148 (2016).

On-target editing efficiencies were predicted by averaging scores generated by the Azimuth model, the DeepSpCas9 model, and the CrisprScan model. The Azimuth model is a boosted regression tree model, trained with 881 sgRNAs (MOLM13/NB4/TF1 cells and additional unpublished data) delivered by lentivirus. Doench, J. G. et al. Optimized sgRNA design to maximize activity and minimize off-target effects of CRISPR-Cas9. Nature Biotechnology 34, 184-191 (2016). DeepSpCas9 is a deep learning model trained using editing data from 12,832 sgRNA. Kim, H. K. et al. SpCas9 activity prediction by DeepSpCas9, a deep learning-based model with high generalization performance. Science Advances 5 (11) (2019). CrisprScan is a linear regression model, trained using editing data from 1000 sgRNAs injected into zebrafish embryos targeting >100 genes. Moreno-Mateos, M. A. et al. CRISPRscan: designing highly efficient sgRNAs for CRISPR-Cas9 targeting in vivo. Nature Methods 12, 982-988 (2015). The column labelled “ON” shows the mean of the scores provided by the three models.

The putative guides' potential to generate frameshift mutations were predicted by averaging scores generated by the Lindel model and the InDelphi model. Lindel is a machine learning model trained using profile data of 1.16 million independent mutational events triggered by CRISPR/Cas9-mediated cleavage and non-homologous end joining-mediated double strand break repair of 6872 synthetic target sequences, introduced into a human cell line via lentiviral infection. Chen, W. et al. Massively parallel profiling and predictive modeling of the outcomes of CRISPR/Cas9-mediated double-strand break repair. Nucleic Acids Research 47, 7989-8003 (2019). InDelphi is machine learning model trained with indels generated by 1872 sgRNAs. Shen, M. W. et al. Predictable and precise template-free CRISPR editing of pathogenic variants. Nature 563, 646-651 (2018). The column labelled “Frameshift” shows the mean of the scores provided by the two models. The column labelled “Precision” shows the frequency distribution of indels estimated by inDelphi. High precision is closer to 100 and represents sgRNAs that are characterized by one or a very low number of repair outcomes.

The candidate spacer sequences were then evaluated for the presence of Graf motifs, TT or GCC present in the 4 PAM proximal bases of the spacer sequence, as indicated in, e.g., FIGS. 26-42, as either TT or GCC. Graf, R. et al. sgRNA Sequence Motifs Blocking Efficient CRISPR/Cas9-Mediated Gene Editing. Cell Reports, 26 (5), 1098-103 (2019). Graf et al. reported that TT-and GCC-motifs are a hallmark of inefficient sgRNAs. If possible, spacer sequences with Graf motifs and in particular the GCC motif were avoided. In contrast to the TT motif, the GCC motif remains critical if sgRNAs are synthesized de novo rather than by transcription.

As shown in, e.g., FIGS. 1-3, three consensus scores were then calculated for each spacer sequence. The “ON-OFF” score represents the mean of the “ON” and “OFF” score. The “OFF-FS” score represents the mean of the “OFF” and “Frameshift” score. The OVERALL score represents the mean of the “ON”, “OFF” and “Frameshift” score. Additionally, for human only, VBC's Bioscore was used to predict whether a possible in-frame mutation could disturb protein function. This is more likely to occur in conserved genomic DNA sequences coding for critical protein domains. Thus, Bioscore is based on protein domain annotations, phylogenetic conservation, amino acid identities and exon size. Michlits, G. et al. Multilayered VBC score predicts sgRNAs that efficiently generate loss-of-function alleles. Nature Methods 17, 708-716 (2020). The final criteria for selecting candidates is mainly based on the OVERALL score (in most cases >70), the relative low counts of potential off-targets (in most cases <200 off-targets), the absence of Graf motifs (if possible) and the genomic cut position within the coding sequence to produce knockouts or, in the case of hTGFBR1 and hTGFBR2, truncated proteins with reduced functionality (e.g. decoy receptors).

Finally, the CRISPR-targeted domains were determined based on the nucleotide position in the gene (FIGS. 4A, 5A, 6A). The domain location is used to predict whether the edit will result in a wild-type like protein (edits in the sequence encoding the extreme C-terminus of a receptor that are unlikely to disrupt receptor-mediated signaling), complete knock-out of any functional protein (edits in the sequence encoding the N-terminus and/or essential functional domains), and—if applicable-a soluble decoy receptor (edits in the sequence encoding the transmembrane domain of the receptor) or a membrane-bound decoy receptor (edits in the sequence encoding the intracellular signaling domain of the receptor).

Indeed, this in silico analysis established that several candidate sgRNAs were predicted to yield genomic edits that would create a dominant negative (DN) TGFB receptor, thereby impacting intracellular pro-inflammatory signaling. These predictive studies provided a high-throughput method for validating candidates without the need for time- and resource-intensive brute force methods and demonstrated the feasibility of predicting the induction of a genetic knockout (or dominant negative TGFB receptor) as a unique approach to treating musculoskeletal fibrosis and/or scarring.

### Example 2: Validation of Tgfb1 Editing in Human Cells

Having designed and analyzed numerous sgRNAs that were predicted to edit TGFB 1 to produce a genetic knockout by bioinformatic methods described in Example 1 (see, FIG. 1), the next step was to assess their ability to act in vitro. Of particular interest is whether the one preferred edit would be observed.

To test this, 80 pmoles of sgRNA candidates were introduced into THP-1 human monocytes via electroporation as part of a ribonucleoprotein (i.e., pre-assembled with 25 pmoles of Cas9 protein at room temperature for at least 5 min). In brief, around 400,000 cells resuspended in 20 μl SG buffer (Lonza) were added to the pre-assembled Cas9 ribonucleoprotein complex, transferred into 16-well nucleocuvettes and electroporated with pulse code FF-100 using the 4D nucleofector (Lonza). Electroporated cells were kept in SG buffer for about 10 min before transferring them into 12-well tissue culture plates with complete culture media. After 2-5 days in culture, cells were dissociated for genomic DNA extraction using the DNeasy Blood & Tissue Kit (Qiagen). sgRNA target regions were amplified by PCR and editing efficacy was then deduced from Sanger sequencing traces using Inference of CRISPR Edits (ICE v1.2). See, Conant, D. et al. (2022). The CRISPR Journal, 5 (1), 123-130.

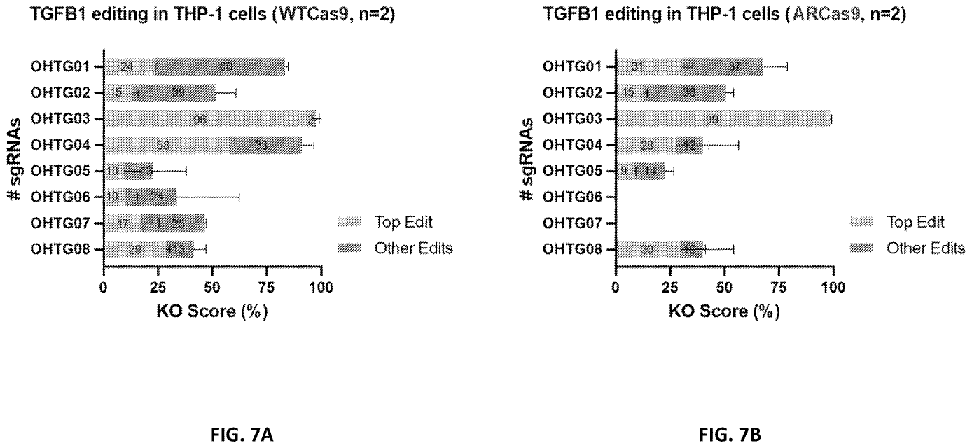

While multiple sgRNA candidates were able to induce mutations in the hTGFB1 gene at high efficiency, with several exhibiting frequencies above 50%, only one candidate-OHTG03 (SEQ ID NO: 682)-exhibited the ability to induce the same edit in a high percentage of cells when paired with either wildtype (WT) Cas9 or high-fidelity AR-Cas9 (IDT, Cat #IDT, Catalogue #1081060; Vakulskas, C. A. et al. (2018). Nature Medicine, 24 (8): 1216-1224) (FIG. 7A, B). It is expected that the use of AR-Cas9 will improve editing specificity (i.e., mitigating off-target editing) as compared to WT-Cas9. These results generally confirmed the in-silico analysis described in Example 1.

### Example 3: Validation of Tgfbr1 Editing in Human Cells

Having designed and analyzed numerous sgRNAs that were predicted to edit the TGFBR1 receptor to produce either a knockout or (soluble or membrane-bound) dominant negative decoy by bioinformatic methods described in Example 1 (see, FIG. 2), the next step was to assess their ability to act in vitro. Of particular interest was whether the one preferred, on-target edit would be observed in any of the candidates.

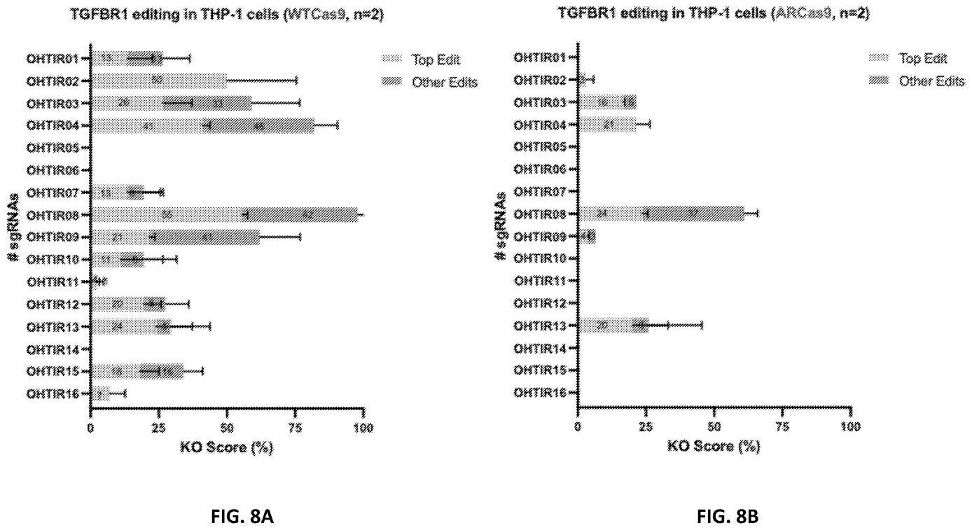

To test this, the sgRNA candidates were complexed with a Cas9 nuclease to form a ribonucleoprotein (RNP) complex before electroporating human THP-1 monocytes to introduce the RNPs to the cells as previously described above. Editing efficacy was then measured via Sanger sequencing. While multiple candidates were able to induce mutations in the hTGFBR1 gene at high efficiency, with several exhibiting frequencies above 50% with WT-Cas9 (FIG. 8A), this efficacy was universally reduced with use of AR-Cas9 (FIG. 8B). These results generally confirmed the in-silico analysis and suggested that several sgRNA candidates, including OHTIR04, were able to efficiently edit TGFBR1 in human cells at some frequency.

### Example 4: Validation of Tgfbr2 Editing in Human Cells

Having designed and analyzed numerous sgRNAs that were predicted to edit the TGFBR2 receptor to produce either a knockout or (soluble or membrane-bound) dominant negative decoy by bioinformatic methods described in Example 1 (see, FIG. 3), the next step was to assess their ability to act in vitro. Of particular interest was whether the one preferred edit would be observed in any of the candidates.

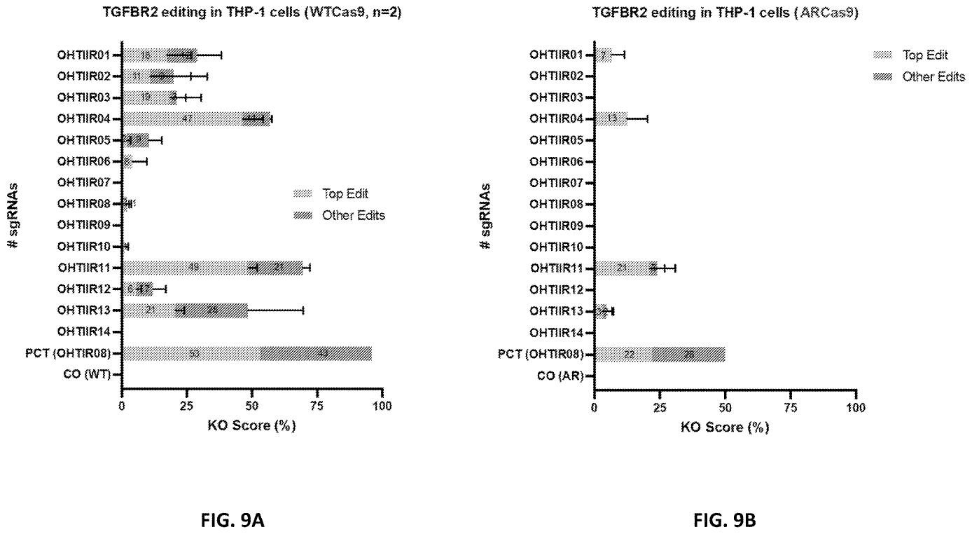

To test this, the sgRNA candidates were complexed with a Cas9 nuclease to form a ribonucleoprotein (RNP) complex before electroporating human THP-1 monocytes to introduce the RNPs to the cells as described above. Editing efficiency was then measured via Sanger sequencing. While multiple candidates were able to induce mutations in the hTGFBR2 gene at high efficacy, with several exhibiting frequencies near or above 50% with WT-Cas9 (FIG. 9A), this efficacy was universally reduced with use of the AR-Cas9 (FIG. 9B). Notably, pairing AR-Cas9 with OHTIIR11 (SEQ ID NO: 1065) resulted in increased editing primacy (i.e., proportion of the editing comprised by the desired on-target edit), albeit at a lower editing efficacy than with WT-Cas9. These results generally confirmed the in-silico analysis and suggested that several sgRNA candidates, including OHTIIR11, were able to efficiently edit TGFBR2 in human cells at some frequency.

### Example 5: Editing of Tgfbr1 or Tgfbr2 Renders Human Monocytes Unresponsive to Tgf-Beta Stimuli

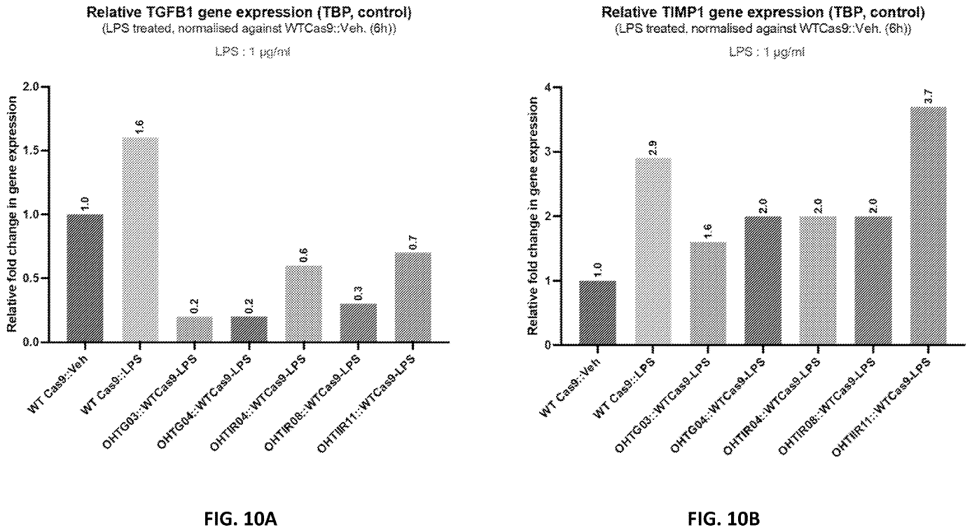

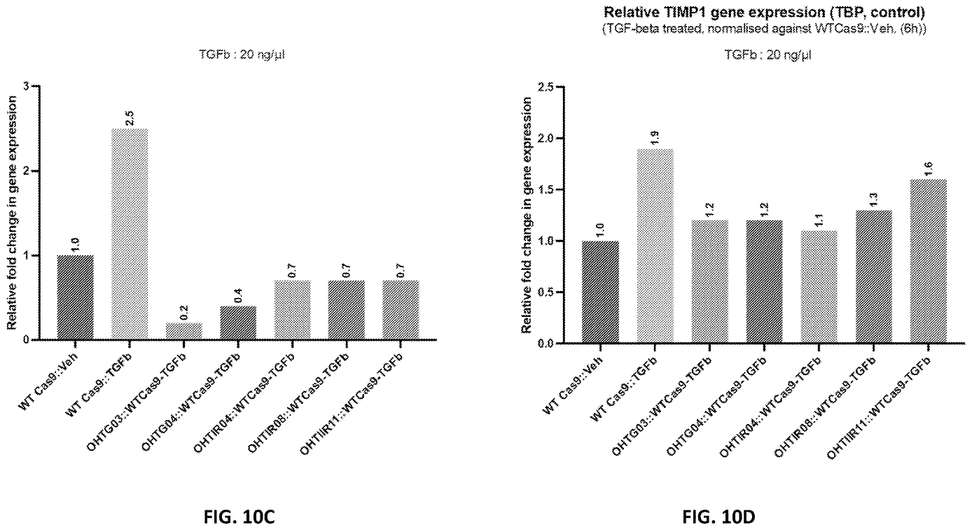

Having designed and validated numerous guides targeting the TGF-beta pathway, the next step involved functional testing of edited cells. As part of this testing, around 400,000 edited human THP-1 monocytes were treated with either 1 μg/ml LPS or 20 ng/μl recombinant TGF-beta for 6 hours. Reverse transcription of total cellular RNA followed by quantitative PCR (RT-qPCR) was carried out to verify whether human either LPS or TGF-beta can stimulate the transcription of TGFB1 as has been previously reported. See, generally, Hall, M. C., et al. (2003). Journal of Biological Chemistry, 278 (12), 10304-10313; Xiang Yin, et al 2017, J Atheroscler Thromb, 2017; 24:55-67. doi: 10.5551/jat.35204. LPS treatment caused a modest increase in TGFB1 transcription in control cells (WT-Cas9 injection only) (FIG. 10A). However, TGFB1 transcription levels were reduced as much as five-fold for TGFBR1-edited cells compared to the unedited control, while TGFBR2-edited cells exhibited a two-fold reduction.

As an additional test, the same cellular RNA was assayed for induction of TIMP1, which canonically is upregulated by TGF-beta signaling via the SMAD proteins (see generally, Hall, M. C., et al. (2003). Journal of Biological Chemistry, 278 (12), 10304-10313.). In the LPS-treated cells, robust induction of TIMP1 was observed in the unedited control as compared to the untreated cells (FIG. 10B). TGFBR1-edited monocytes demonstrated a one-fold decrease in TIMP1 induction as compared to the unedited control. There was no change for TGFBR2-edited cells.

Results were even more striking following treatment with the natural ligand. In response to TGF-beta treatment, TGFB1 induction was more robust in unedited WTCas9 only injected cells, and edits to either TGFBR1 or TGFBR2 reduced this response by three-fold (FIG. 10C). These results were similar to those observed for TIMP1 induction following TGF-beta treatment (FIG. 10D). These results collectively showed that the edited cells did not respond to TGF-beta treatment, as their levels of TGFB 1 remained well below the control. Taken together, these results demonstrated that TGF-beta positive feedback (TGFB 1 induction) and downstream signaling (TIMP1 induction) were both impaired in cells with edited TGFBR1 and TGFBR2.

### Example 6: Validation of Tgfb1 Editing in Human Cells

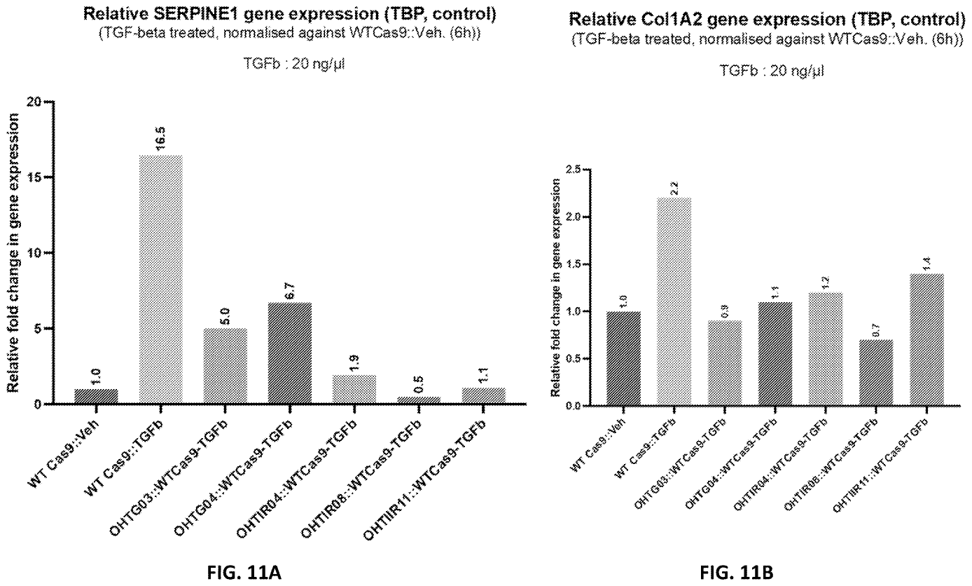

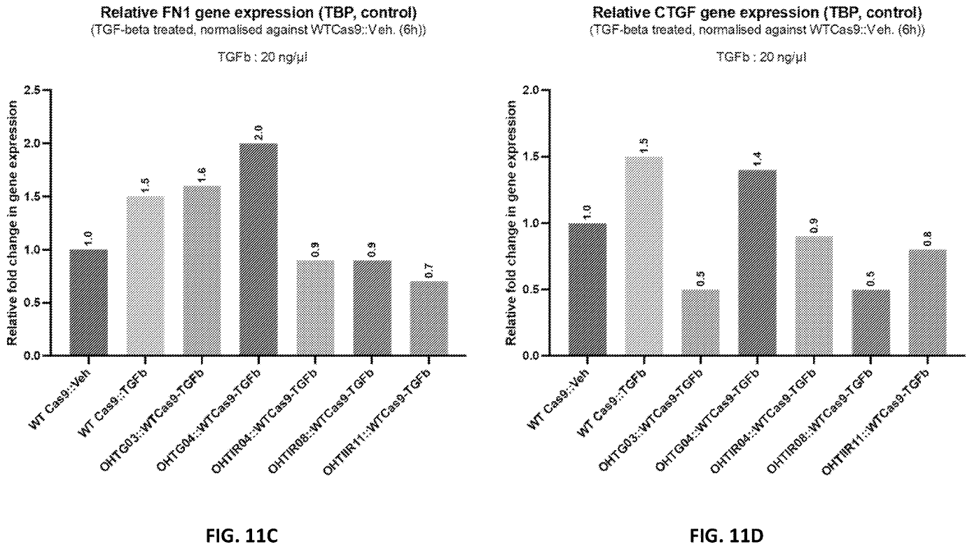

Having designed and analyzed numerous sgRNA candidates that were predicted to edit the human TGFB1 gene and produce either a genetic knockout (see FIG. 11A), the next step is to assess their ability to act in vitro. Of particular interest is whether the preferred (i.e., on-target) edit will be observed in edited cells.

To test this, sgRNA candidates (SEQ ID NOS: 1-198) are introduced into human THP-1 monocytes via electroporation as part of a ribonucleoprotein as previously described in Example 2.

It is anticipated that results of these studies will further confirm the robustness of the in silico analysis and identify preferred candidates that are indeed able to induce mutations in the TGFB1 gene at high efficacy. It is further expected that the use of high-fidelity AR-Cas9 will improve editing specificity (i.e., mitigating off-target editing) as compared to WT-Cas9.

### Example 7: In Vitro Delivery of Tgfb1-Editing Guide RNA to Human Cells Via Lipid Nanoparticles

Additional experiments will assess the suitability of using lipid nanoparticle (LNP) to deliver mRNA to human cells. LNPs containing sgRNA (SEQ ID NOS: 1-198) and WT- or AR-Cas9 mRNA are exposed to THP-1 monocytes for 8 to 24 hours under typical cell culture conditions. Sanger sequencing as described in Example 2 will be used to validate editing. Upon confirmation of editing various functional assays as described in Example 5 will be performed.

### Example 8: In Vitro Delivery of Tgfb1-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells.

TABLE 2
Exemplary AAV vectors for introduction of sgRNA to human cells.

AAVPrimeTM Adeno-Associated Virus - Serotype6 (AAV-6) Particles for
sgRNA (GeneCopoeia ™, catalog No. AA06-MCP001682-AD01-2-200-a, lot No.
GC03182K2001)
AAVPrimeTM Adeno-Associated Virus - Serotype6 (AAV-6) Particles for
sgRNA (GeneCopoeia ™, catalog No. AA06-MCP001682-AD01-2-200-b, lot No.
GC03202K2001)
AAVPrimeTM Adeno-Associated Virus - Serotype6 (AAV-6) Particles for
sgRNA (GeneCopoeia ™, catalog No. AA06-CCPCTR01-AD01-200, lot No.
GC03112K2002)
AAVPrimeTM Adeno-Associated Virus - Serotype5 (AAV-5) Particles for
sgRNA (GeneCopoeia ™, catalogue No. AA05-MCP001682-AD01-2-200A, lot No. GC-
03182K2002)
AAVPrimeTM Adeno-Associated Virus - Serotype5 (AAV-5) Particles for
sgRNA (GeneCopoeia ™, catalogue No. AA05-MCP001682-AD01-2-200B, lot No.
GC03182K2003)
AAVPrimeTM Adeno-Associated Virus - Serotype5 (AAV-5) Particles for
sgRNA (GeneCopoeiaTM, catalogue No. AA05-CCPCTR01-AD01-200, lot No.
GC03032K2003)

Exemplary AAV vectors are shown in Table 2. After being prepared according to manufacturer specifications, such that one or more of the sgRNAs containing SEQ ID NOS: 1-198 are integrated into the viral vector, said vectors are incubated (MOI-0.1 to 10) with approximately 500,000 U2OS cells that have been stably transfected with Cas9 (see, e.g., Rojas-Fernandez, A., et al. (2015). Scientific Reports, 5 (1), 1-6.). At 2-7 days post-infection, Sanger sequencing as described in Example 2 will be used to validate editing. Upon confirmation of editing, various functional assays as described in Example 5 will be performed.

### Example 9: Validation of Tgfbr1 Editing in Human Cells

Having designed and analyzed numerous sgRNA candidates that were predicted to edit the human Transforming Growth Factor Beta Receptor 1 protein to produce either a knockout or a dominant negative receptor (see FIG. 11B), the next step is to assess their ability to act in vitro. Of particular interest is whether the preferred (i.e., on-target) edit will be observed in edited cells.

To test this, sgRNA candidates (SEQ ID NOS: 199-320) are introduced into human THP-1 monocytes via electroporation as part of a ribonucleoprotein as previously described in Example 2.

It is anticipated that results of these studies will further confirm the robustness of the in silico analysis and identify multiple preferred candidates that are indeed able to induce mutations in the TGFBR1 gene at high efficacy. It is further expected that the use of high-fidelity AR-Cas9 will improve editing specificity (i.e., mitigating off-target editing) as compared to WT-Cas9.

### Example 10: In Vitro Delivery of Tgfbr1-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOS: 199-320) or Cas9 mRNA are incubated with THP-1 monocytes, followed by validation of editing as described in Example 7. Upon confirmation of editing various functional assays as described in Example 5 will be performed.

### Example 11: In Vitro Delivery of Tgfbr1-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOS: 199-320 are integrated into the viral vector, said vectors are introduced to human U2OS cells and editing is validated as previously described in Example 8. Upon confirmation of editing, various functional assays as described in Example 5 will be performed.

### Example 12: Validation of Tgfbr2 Editing in Human Cells

Having designed and analyzed numerous sgRNA candidates that were predicted to edit the human Transforming Growth Factor Beta Receptor 2 protein to produce either a knockout or a dominant negative receptor (see FIG. 11C), the next step is to assess their ability to act in vitro. Of particular interest is whether the preferred (i.e., on-target) edit will be observed in edited cells.

To test this, sgRNA candidates (SEQ ID NOS: 321-519) are introduced into human THP-1 monocytes via electroporation as part of a ribonucleoprotein as previously described in Example 2.

It is anticipated that results of these studies will further confirm the robustness of the in silico analysis and identify multiple preferred candidates that are indeed able to induce mutation in the TGFBR2 gene at high efficacy. It is further expected that the use of high-fidelity AR-Cas9 will improve editing specificity (i.e., mitigating off-target editing) as compared to WT-Cas9.

### Example 13: In Vitro Delivery of Tgfbr2-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOS: 321-519) or Cas9 mRNA are incubated with THP-1 monocytes, followed by validation of editing as described in Example 7. Upon confirmation of editing various functional assays as described in Example 5 will be performed.

### Example 14: In Vitro Delivery of Tgfbr2-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOS: 321-519 are integrated into the viral vector, said vectors are introduced to human U2OS cells and editing is validated as previously described in Example 8. Upon confirmation of editing, various functional assays as described in Example 5 will be performed.

### Example 15: Post-Surgical Treatment of Arthrofibrosis

Additional experiments will be performed to demonstrate the efficacy of arthrofibrosis treatment in vivo in a post-surgical setting. Briefly, compositions of pre-assembled RNP complexes comprising sgRNAs directed to the rabbit TGFB1, TGFBR1, and TGFBR2 genes and a Cas9 enzyme will be prepared as described in Example 2. These RNPs will be injected intraarticularly to rabbits exhibiting arthrofibrosis.

Briefly, two cohorts of rabbits will undergo contracture-forming surgery with knee immobilization followed by remobilization surgery at eight weeks. At remobilization, on cohort will undergo capsular release, while the other cohort will not. Each cohort will then be split into sub-cohorts. For each cohort, one sub-cohort will receive injections of the RNPs prepared above, while the other sub-cohort will not. Evaluation for clinical symptoms of arthrofibrosis will then be performed at several time points following injection, e.g., 10, 16, and 24 weeks, and disarticulated limbs will be evaluated for fibrosis at the end of the study. An example of a study using this rabbit model of arthrofibrosis is described in Trousdale et al. Bone Joint Res., 2022, 11 (1): 32-39, doi: 10.1302/2046-3758.111.BJR-2021-0546.R1.

### Example 16: Editing of Tgfb1, Tgfbr1, or Tgfbr2 Renders Human Monocytes Unresponsive to Drive Expression of Multiple Tgf-Beta-Responsive Gene Products

Additional functional studies were performed on edited and control THP-1 monocytes, in which TGF-beta was added for 6 hours followed by collection of total cellular RNA for RT-qPCR analysis, as described above in Example 5. SERPINE1 demonstrated strong induction as a result of TGF-beta treatment compared to the unedited vehicle control cells (FIG. 11A), while all edited cells saw strongly reduced induction, particularly the TGFBR1- and TGFBR2-edited monocytes. Similarly, Col1A2 was induced by TGF-beta treatment, and all edited monocytes demonstrated an approximately two-fold decrease in induction (FIG. 11B). Notably, less robust induction was observed for FN1 (FIG. 11C) and CTGF (FIG. 11D). As such, the relative induction is less clear in the edited cells, though it should be noted that the TGFBR1- and TGFBR2-edited monocytes exhibited levels of FN1 and CTGF that are commensurate with unedited vehicle control. This result suggested that these cells were incapable of inducing RNA beyond background levels, even after 6 hours of TGF-beta treatment. Collectively, these results showed that several additional TGF-beta-responsive gene products displayed little to no expression above background in TGFBR1-and TGFBR2-edited cells following exposure to TGF-beta, while TGFB1-edited cells also showed strong reductions in the induction of these TGF-beta-responsive gene products.

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

What is claimed is:

1. A pharmaceutical composition for treating or preventing a disorder having a symptom caused, at least in part, by intercellular signaling mediated through the transforming growth factor beta (TGFB) signaling pathway, the composition comprising:
(i) an RNA-guided nuclease or a nucleic acid encoding an RNA-guided nuclease; and
(ii) at least one guide RNA or a nucleic acid encoding at least one guide RNA targeting a TGFB1 gene, TGFBR1 gene, TGFBR2 gene, or a combination thereof.

2. The pharmaceutical composition of claim 1, wherein the disorder is fibrosis.

3. The pharmaceutical composition of claim 2, wherein the fibrosis is postoperative fibrosis.

4. The pharmaceutical composition of claim 2, wherein the fibrosis is post-ligament reconstruction fibrosis.

5. The pharmaceutical composition of claim 4, wherein the fibrosis is post-anterior cruciate ligament (ACL) reconstruction fibrosis, post-autograft ACL reconstruction fibrosis, or post-allograft ACL reconstruction fibrosis.

6. The pharmaceutical composition of claim 2, wherein the fibrosis is knee arthrofibrosis.

7. The pharmaceutical composition of claim 2, wherein the fibrosis is due to intra-articular fibrous nodules.

8. The pharmaceutical composition of claim 2, wherein the fibrosis is post-total knee arthroplasty (TKA).

9. The pharmaceutical composition of claim 2, wherein the fibrosis is related to related to tendon injury or surgery.

10. The pharmaceutical composition of claim 2, wherein the fibrosis is post-fracture repair fibrosis.

11. The pharmaceutical composition of claim 2, wherein the fibrosis is post microdiscectomy fibrosis.

12. The pharmaceutical composition of claim 2, wherein the fibrosis is post-microdiscectomy epidural fibrosis.

13. The pharmaceutical composition of claim 2, wherein the fibrosis is post-lumbar laminectomy fibrosis.

14. The pharmaceutical composition of claim 2, wherein the fibrosis is post-lumbar laminectomy epidural fibrosis.

15. The pharmaceutical composition of claim 2, wherein the fibrosis is post-anterior acromioplasty fibrosis.

16. The pharmaceutical composition of claim 2, wherein the fibrosis is post-subacromial decompression fibrosis.

17. The pharmaceutical composition of claim 2, wherein the fibrosis is post-anterior acromioplasty soft tissue fibrosis.

18. The pharmaceutical composition of claim 2, wherein the fibrosis is post-anterior acromioplasty joint contracture.

19. The pharmaceutical composition of claim 2, wherein the fibrosis is post-arthroscopy fibrosis.

20. The pharmaceutical composition of claim 2, wherein the fibrosis is post-arthroscopy arthrofibrosis.

21. The pharmaceutical composition of claim 2, wherein the fibrosis is fibrosis of the trabecular meshwork.

22. The pharmaceutical composition of claim 21, wherein fibrosis of the Trabecular Meshwork is post-glaucoma surgery fibrosis.

23. The pharmaceutical composition of claim 2, wherein the fibrosis is epidural fibrosis.

24. The pharmaceutical composition of claim 2, wherein the fibrosis is atrial fibrosis, cardiac fibrosis, myocardial fibrosis, and/or post-open heart surgery fibrosis.

25. The pharmaceutical composition of claim 2, wherein the fibrosis is post-liposuction fibrosis.

26. The pharmaceutical composition of claim 2, wherein the fibrosis is idiopathic pulmonary fibrosis (IPF).

27. The pharmaceutical composition of claim 2, wherein the fibrosis is fibrosis in a knee joint, a shoulder joint, or an elbow joint.

28. The pharmaceutical composition of claim 1, wherein the disorder includes fibrosis in a kidney tissue.

29. The pharmaceutical composition of claim 28, wherein the disorder is chronic kidney disease.

30. The pharmaceutical composition of claim 1, wherein the disorder includes fibrosis in a skin tissue.

31. The pharmaceutical composition of claim 30, wherein the disorder is scarring post wound healing, keloid disorder, nephrogenic systemic fibrosis, or scleroderma/systemic sclerosis.

32. The pharmaceutical composition of claim 1, wherein the disorder includes fibrosis in a lung tissue.

33. The pharmaceutical composition of claim 32, wherein the disorder is fibrothorax, pulmonary fibrosis, cystic fibrosis, idiopathic pulmonary fibrosis, radiation-induced lung injury, progressive massive fibrosis, or scleroderma/systemic sclerosis.

34. The pharmaceutical composition of claim 1, wherein the disorder includes fibrosis in a liver tissue.

35. The pharmaceutical composition of claim 34, wherein the disorder is cirrhosis or bridging fibrosis.

36. The pharmaceutical composition of claim 1, wherein the disorder includes fibrosis in a cardiac tissue.

37. The pharmaceutical composition of claim 36, wherein the fibrosis is interstitial fibrosis.

38. The pharmaceutical composition of claim 36, wherein the disorder is congestive heart failure or hypertension.

39. The pharmaceutical composition of claim 36, wherein the fibrosis is replacement fibrosis.

40. The pharmaceutical composition of claim 36, wherein the disorder is myocardial infarction.

41. The pharmaceutical composition of claim 1, wherein the disorder includes fibrosis in a brain tissue.

42. The pharmaceutical composition of claim 41, wherein the fibrosis is a glial scar.

43. The pharmaceutical composition of claim 1, wherein the disorder includes fibrosis in an intestinal tissue.

44. The pharmaceutical composition of claim 43, wherein the disorder is Crohn's disease.

45. The pharmaceutical composition of claim 1, wherein the disorder includes fibrosis in a hand or finger.

46. The pharmaceutical composition of claim 45, wherein the disorder is Dupuytren's contracture.

47. The pharmaceutical composition of claim 1, wherein the disorder includes fibrosis in a lymph tissue.

48. The pharmaceutical composition of claim 47, wherein the fibrosis is mediastinal fibrosis.

49. The pharmaceutical composition of claim 1, wherein the disorder includes fibrosis in a bone marrow tissue.

50. The pharmaceutical composition of claim 49, wherein the fibrosis is myelofibrosis.

51. The pharmaceutical composition of claim 1, wherein the disorder includes fibrosis in a penile tissue.

52. The pharmaceutical composition of claim 51, wherein the disorder is Peyronie's disease.

53. The pharmaceutical composition of claim 1, wherein the disorder includes fibrosis in a soft tissue of the retroperitoneum.

54. The pharmaceutical composition of claim 53, wherein the fibrosis is retroperitoneal fibrosis.

55. The pharmaceutical composition of claim 1, wherein the disorder includes fibrosis in a musculoskeletal tissue.

56. The pharmaceutical composition of claim 1, wherein the disorder is a cancer.

57. The pharmaceutical composition of claim 1, wherein the cancer is a musculoskeletal cancer.

58. The pharmaceutical composition of claim 1, wherein the disorder is a musculoskeletal disorder.

59. The pharmaceutical composition of claim 58, wherein the musculoskeletal disorder is a genetic musculoskeletal disease.

60. The pharmaceutical composition of claim 58, wherein the musculoskeletal disorder is a muscular dystrophy.

61. The pharmaceutical composition of claim 58, wherein the musculoskeletal disorder is selected from Duchenne muscular dystrophy (DMD), myotonic dystrophy (DM), facioscapulohumeral muscular dystrophy (FSHD), and limb-girdle muscular dystrophy (LGMD).

62. The pharmaceutical composition of claim 58, wherein the musculoskeletal disorder is a type II collagenopathy.

63. The pharmaceutical composition of claim 58, wherein the musculoskeletal disorder is selected from achondrogenesis type II, hypochondrogenesis, Stickler Syndrome, and Czech Dysplasia.

64. The pharmaceutical composition of claim 58, wherein the musculoskeletal disorder is osteogenesis imperfecta (OI).

65. The pharmaceutical composition of claim 58, wherein the musculoskeletal disorder is an autoimmune musculoskeletal disease.

66. The pharmaceutical composition of claim 58, wherein the musculoskeletal disorder is an autoimmune musculoskeletal disease with polygenic susceptibility traits.

67. The pharmaceutical composition of claim 58, wherein the musculoskeletal disorder is a spondyloarthropathy (SA).

68. The pharmaceutical composition of claim 58, wherein the musculoskeletal disorder is ankylosing spondylitis.

69. The pharmaceutical composition of claim 58, wherein the musculoskeletal disorder is rheumatoid arthritis.

70. The pharmaceutical composition of claim 58, wherein the musculoskeletal disorder is autoimmune osteoarthritis.

71. The pharmaceutical composition of claim 58, wherein the musculoskeletal disorder is osteoarthritis (OA).

72. The pharmaceutical composition of claim 58, wherein the musculoskeletal disorder is Camurati-Engelmann disease.

73. The pharmaceutical composition of claim 58, wherein the musculoskeletal disorder is Sjogren's Syndrome.

74. The pharmaceutical composition of claim 58, wherein the musculoskeletal disorder is a mechanical musculoskeletal injury.

75. The pharmaceutical composition of claim 58, wherein the musculoskeletal disorder is selected from chronic muscle injury, torn tendon, post-traumatic osteoarthritis, and post-traumatic arthrofibrosis.

76. The pharmaceutical composition of any one of claims 1-75, wherein the at least one guide RNA or a nucleic acid encoding at least one guide RNA targets a mammalian TGFB1 gene.

77. The pharmaceutical composition of claim 76, wherein the mammalian TGFB1 gene is a canine, equine, or feline TGFB1 gene.

78. The pharmaceutical composition of claim 76, wherein the mammalian TGFB1 gene is a human TGFB1 gene.

79. The pharmaceutical composition of claim 78, wherein the at least one guide RNA comprises a spacer sequence selected from the group consisting of those sequences shown in FIGS. 1A-1D (SEQ ID NOS: 1-198).

80. The pharmaceutical composition of claim 78, wherein the at least one guide RNA comprises a spacer sequence selected from the group consisting of those sequences shown in FIG. 12A (SEQ ID NOS: 520-527).

81. The pharmaceutical composition of claim 78, wherein the at least one guide RNA comprises a spacer sequence of OHTG03 (SEQ ID NO:522) or OHTG04 (SEQ ID NO: 523).

82. The pharmaceutical composition of any one of claims 1-75, wherein the at least one guide RNA or a nucleic acid encoding at least one guide RNA targets a mammalian TGFBR1 gene.

83. The pharmaceutical composition of claim 82, wherein the mammalian TGFBR1 gene is a canine, equine, or feline TGFBR1 gene.

84. The pharmaceutical composition of claim 82, wherein the mammalian TGFBR1 gene is a human TGFBR1 gene.

85. The pharmaceutical composition of claim 84, wherein the at least one guide RNA comprises a spacer sequence selected from the group consisting of those sequences shown in FIGS. 2A-2C (SEQ ID NOS: 199-320).

86. The pharmaceutical composition of claim 84, wherein the at least one guide RNA comprises a spacer sequence selected from the group consisting of those sequences shown in FIG. 12B (SEQ ID NOS: 528-552).

87. The pharmaceutical composition of claim 84, wherein the at least one guide RNA comprises a spacer sequence selected from the group consisting of those sequences shown in FIG. 5B.

88. The pharmaceutical composition of claim 84, wherein the at least one guide RNA comprises a spacer sequence of OHTIR04 (SEQ ID NO:532) or OHTIR08 (SEQ ID NO: 539).

89. The pharmaceutical composition of any one of claims 1-75, wherein the at least one guide RNA or a nucleic acid encoding at least one guide RNA targets a mammalian TGFBR2 gene.

90. The pharmaceutical composition of claim 89, wherein the mammalian TGFBR2 gene is a canine, equine, or feline TGFBR2 gene.

91. The pharmaceutical composition of claim 89, wherein the mammalian TGFBR2 gene is a human TGFBR2 gene.

92. The pharmaceutical composition of claim 91, wherein the at least one guide RNA comprises a spacer sequence selected from the group consisting of those sequences shown in FIGS. 3A-3D (SEQ ID NOS: 321-519).

93. The pharmaceutical composition of claim 91, wherein the at least one guide RNA comprises a spacer sequence selected from the group consisting of those sequences shown in FIG. 12C (SEQ ID NOS: 553-604).

94. The pharmaceutical composition of claim 91, wherein the at least one guide RNA comprises a spacer sequence selected from the group consisting of those sequences shown in FIG. 6B.

95. The pharmaceutical composition of claim 91, wherein the at least one guide RNA comprises a spacer sequence of OHTIIR04 (SEQ ID NO:563).

96. The pharmaceutical composition of any one of claims 1-95, wherein the RNA-guided nuclease or a nucleic acid encoding an RNA-guided nuclease is the RNA-guided nuclease.

97. The pharmaceutical composition of any one of claims 1-95, wherein the RNA-guided nuclease or a nucleic acid encoding an RNA-guided nuclease is DNA encoding the RNA-guided nuclease.

98. The pharmaceutical composition of any one of claims 1-95, wherein the RNA-guided nuclease or a nucleic acid encoding an RNA-guided nuclease is mRNA encoding the RNA-guided nuclease.

99. The pharmaceutical composition of any one of claims 1-98, wherein the RNA-guided nuclease is a Cas protein.

100. The pharmaceutical composition of claim 99, wherein the Cas protein is a Cas9 protein.

101. The pharmaceutical composition of claim 99, wherein the Cas9 protein is an S. pyogenes Cas9 polypeptide.

102. The pharmaceutical composition of claim 99, wherein the Cas9 protein is selected from the group consisting of ESCas9, HFCas9, PECas9, and ARCas9.

103. The pharmaceutical composition of any one of claims 1-102, wherein the at least one guide RNA or a nucleic acid encoding at least one guide RNA is the at least one guide RNA.

104. The pharmaceutical composition of any one of claims 1-102, wherein the at least one guide RNA or a nucleic acid encoding at least one guide RNA is DNA encoding the at least one guide RNA.

105. The pharmaceutical composition of any one of claims 1-102, comprising a nucleic acid encoding both the RNA-guided nuclease and the at least one guide RNA.

106. The pharmaceutical composition of any one of claims 1-105, wherein the at least one guide RNA is a single guide RNA (sgRNA).

107. The pharmaceutical composition of any one of claims 1-106, wherein the composition comprises one or more viral vectors collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

108. The pharmaceutical composition of claim 107, wherein the one of more viral vectors comprise a recombinant virus selected from a retrovirus, an adenovirus, an adeno-associated virus, a lentivirus, and a herpes simplex virus-1.

109. The pharmaceutical composition of claim 107, wherein the one of more viral vectors comprise a recombinant adeno-associated virus (AAV).

110. The pharmaceutical composition of claim 109, wherein the recombinant AAV is of serotype 5 (AAV5).

111. The pharmaceutical composition of claim 109, wherein the recombinant AAV is of serotype 6 (AAV6).

112. The pharmaceutical composition of any one of claims 1-106, wherein the composition comprises one or more lipid nanoparticles (LNP) collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

113. The pharmaceutical composition of claim 112, wherein the one or more LNP comprises:
a first plurality of LNP encapsulating the RNA-guided nuclease or a nucleic acid encoding an RNA-guided nuclease; and
a second plurality of LNP encapsulating the at least one guide RNA or a nucleic acid encoding at least one guide RNA.

114. The pharmaceutical composition of claim 112, wherein the one or more LNP comprises a plurality of LNP encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

115. The pharmaceutical composition of any one of claims 112-114, wherein the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

116. The pharmaceutical composition of any one of claims 112-115, wherein the LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

117. The pharmaceutical composition of any one of claims 112-116, wherein the LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

118. The pharmaceutical composition of any one of claims 112-117, wherein the LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9, 11-dehydroergosterol, tomatidine, tomatine, a-tocopherol, and a mixture thereof.

119. The pharmaceutical composition of any one of claims 112-114, wherein the one or more LNP comprises an LP01 ionizable lipid, a neutral/helper lipid component, a steroid system component, and a pegylated lipid component.

120. The pharmaceutical composition of any one of claims 112-114, wherein the one or more LNP comprises an SM-102 ionizable lipid, a neutral/helper lipid component, a steroid system component, and a pegylated lipid component.

121. The pharmaceutical composition of any one of claims 112-114, wherein the one or more LNP comprises an ALC0315 ionizable lipid, a neutral/helper lipid component, a steroid system component, and a pegylated lipid component.

122. The pharmaceutical composition of any one of claims 119-121, wherein the neutral/helper lipid component comprises DSPC.

123. The pharmaceutical composition of claim 119-122, wherein the steroid system component comprises cholesterol.

124. The pharmaceutical composition of claim 119-123, wherein the steroid system component comprises a glucocorticoid steroid.

125. The pharmaceutical composition of claim 124, wherein the glucocorticoid steroid is dexamethasone.

126. The pharmaceutical composition of claim 119-125, wherein the pegylated lipid component comprises DMG-PEG.

127. The pharmaceutical composition of claim 126, wherein the DMG-PEG is DMG-PEG2000.

128. The pharmaceutical composition of any one of claims 119-127, wherein the one or more LNP comprises LP01, a neutral/helper lipid component, a steroid system component, and a pegylated lipid component at a molar ratio of (43-47): (7.5-10.5): (42-46): (1.5-2.5).

129. The pharmaceutical composition of any one of claims 119-127, wherein the one or more LNP comprises SM-102, a neutral/helper lipid component, a steroid system component, and a pegylated lipid component at a molar ratio of (48-52): (8.5-11.5): (36.5-40.5): (1-2).

130. The pharmaceutical composition of any one of claims 119-127, wherein the one or more LNP comprises ALC0315, a neutral/helper lipid component, a steroid system component, and a pegylated lipid component at a molar ratio of (48-52): (8.5-11.5): (36.5-40.5): (1-2).

131. The pharmaceutical composition of any one of claims 1-106, wherein the composition comprises one or more liposomes collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

132. The pharmaceutical composition of claim 131, wherein the one or more liposomes comprises:
a first plurality of liposomes encapsulating the RNA-guided nuclease or a nucleic acid encoding an RNA-guided nuclease; and
a second plurality of liposomes encapsulating the at least one guide RNA or a nucleic acid encoding at least one guide RNA.

133. The pharmaceutical composition of claim 131, wherein the one or more liposomes comprises a plurality of liposomes encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

134. The pharmaceutical composition of any one of claims 1-106, wherein the composition comprises one or more virus-like particles collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

135. The pharmaceutical composition of claim 134, wherein the one or more virus-like particles comprises:
a first plurality of virus-like particles encapsulating the RNA-guided nuclease or a nucleic acid encoding an RNA-guided nuclease; and
a second plurality of virus-like particles encapsulating the at least one guide RNA or a nucleic acid encoding at least one guide RNA.

136. The pharmaceutical composition of claim 134, wherein the one or more virus-like particles comprises a plurality of virus-like particles encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

137. The pharmaceutical composition of any one of claims 1-136, wherein the composition is formulated for parenteral administration.

138. The pharmaceutical composition of any one of claims 1-136, wherein the composition is formulated for intra-articular injection within a joint of the subject.

139. The pharmaceutical composition of any one of claims 1-136, wherein the composition is formulated for intradiscal injection.

140. The pharmaceutical composition of any one of claims 1-136, wherein the composition is formulated for peridiscal injection.

141. The pharmaceutical composition of any one of claims 1-136, wherein the composition is formulated for intravertebral injection.

142. A method for treating or preventing a disorder having a symptom caused, at least in part, by intercellular signaling mediated through the transforming growth factor beta (TGFB) signaling pathway in a subject in need thereof, comprising:
administering a therapeutically effective amount of a composition, wherein the composition comprises:
(i) an RNA-guided nuclease or a nucleic acid encoding an RNA-guided nuclease; and
(ii) at least one guide RNA or a nucleic acid encoding at least one guide RNA targeting a TGFB1 gene, TGFBR1 gene, TGFBR2 gene, or a combination thereof.

143. The method of claim 142, wherein the disorder is fibrosis.

144. The method of claim 143, wherein the fibrosis is postoperative fibrosis.

145. The method of claim 143, wherein the fibrosis is post-ligament reconstruction fibrosis.

146. The method of claim 145, wherein the fibrosis is post-anterior cruciate ligament (ACL) reconstruction fibrosis, post-autograft ACL reconstruction fibrosis, or post-allograft ACL reconstruction fibrosis, or fibrosis post-tendon injuriy or repair.

147. The method of claim 143, wherein the fibrosis is knee arthrofibrosis.

148. The method of claim 143, wherein the fibrosis is due to intra-articular fibrous nodules.

149. The method of claim 143, wherein the fibrosis is post-total knee arthroplasty (TKA).

150. The pharmaceutical composition of claim 2, wherein the fibrosis is post-fracture repair fibrosis.

151. The method of claim 143, wherein the fibrosis is post-TKA knee arthrofibrosis.

152. The method of claim 143, wherein the fibrosis is post microdiscectomy fibrosis.

153. The method of claim 143, wherein the fibrosis is post-microdiscectomy epidural fibrosis.

154. The method of claim 143, wherein the fibrosis is post-lumbar laminectomy fibrosis.

155. The method of claim 143, wherein the fibrosis is post-lumbar laminectomy epidural fibrosis.

156. The method of claim 143, wherein the fibrosis is post-anterior acromioplasty fibrosis.

157. The method of claim 143, wherein the fibrosis is post-subacromial decompression fibrosis.

158. The method of claim 143, wherein the fibrosis is post-anterior acromioplasty soft tissue fibrosis.

159. The method of claim 143, wherein the fibrosis is post-anterior acromioplasty joint contracture.

160. The method of claim 143, wherein the fibrosis is post-arthroscopy fibrosis.

161. The method of claim 143, wherein the fibrosis is post-arthroscopy arthrofibrosis.

162. The method of claim 143, wherein the fibrosis is fibrosis of the trabecular meshwork.

163. The method of claim 162, wherein fibrosis of the Trabecular Meshwork is post-glaucoma surgery fibrosis.

164. The method of claim 143, wherein the fibrosis is epidural fibrosis.

165. The method of claim 143, wherein the fibrosis is atrial fibrosis, cardiac fibrosis, myocardial fibrosis, and/or post-open heart surgery fibrosis.

166. The method of claim 143, wherein the fibrosis is post-liposuction fibrosis.

167. The method of claim 143, wherein the fibrosis is idiopathic pulmonary fibrosis (IPF).

168. The method of claim 143, wherein the fibrosis is fibrosis in a knee joint, a shoulder joint, or an elbow joint.

169. The method of claim 142, wherein the disorder includes fibrosis in a kidney tissue.

170. The method of claim 142, wherein the disorder is chronic kidney disease.

171. The method of claim 142, wherein the disorder includes fibrosis in a skin tissue.

172. The method of claim 142, wherein the disorder is scarring post wound healing, keloid disorder, nephrogenic systemic fibrosis, or scleroderma/systemic sclerosis.

173. The method of claim 142, wherein the disorder includes fibrosis in a lung tissue.

174. The method of claim 142, wherein the disorder is fibrothorax, pulmonary fibrosis, cystic fibrosis, idiopathic pulmonary fibrosis, radiation-induced lung injury, progressive massive fibrosis, or scleroderma/systemic sclerosis.

175. The method of claim 142, wherein the disorder includes fibrosis in a liver tissue.

176. The method of claim 142, wherein the disorder is cirrhosis or bridging fibrosis.

177. The method of claim 142, wherein the disorder includes fibrosis in a cardiac tissue.

178. The method of claim 143, wherein the fibrosis is interstitial fibrosis.

179. The method of claim 142, wherein the disorder is congestive heart failure or hypertension.

180. The method of claim 143, wherein the fibrosis is replacement fibrosis.

181. The method of claim 142, wherein the disorder is myocardial infarction.

182. The method of claim 142, wherein the disorder includes fibrosis in a brain tissue.

183. The method of claim 143, wherein the fibrosis is a glial scar.

184. The method of claim 142, wherein the disorder includes fibrosis in an intestinal tissue.

185. The method of claim 142, wherein the disorder is Crohn's disease.

186. The method of claim 142, wherein the disorder includes fibrosis in a hand or finger.

187. The method of claim 142, wherein the disorder is Dupuytren's contracture.

188. The method of claim 142, wherein the disorder includes fibrosis in a lymph tissue.

189. The method of claim 143, wherein the fibrosis is mediastinal fibrosis.

190. The method of claim 142, wherein the disorder includes fibrosis in a bone marrow tissue.

191. The method of claim 143, wherein the fibrosis is myelofibrosis.

192. The method of claim 142, wherein the disorder includes fibrosis in a penile tissue.

193. The method of claim 142, wherein the disorder is Peyronie's disease.

194. The method of claim 142, wherein the disorder includes fibrosis in a soft tissue of the retroperitoneum.

195. The method of claim 143, wherein the fibrosis is retroperitoneal fibrosis.

196. The method of claim 142, wherein the disorder is a cancer.

197. The method of claim 196, wherein the cancer is a musculoskeletal cancer.

198. The method of claim 142, wherein the disorder includes fibrosis in a musculoskeletal tissue.

199. The method of claim 142, wherein the disorder is a musculoskeletal disorder.

200. The method of claim 199, wherein the musculoskeletal disorder is a genetic musculoskeletal disease.

201. The method of claim 199, wherein the musculoskeletal disorder is a muscular dystrophy.

202. The method of claim 199, wherein the musculoskeletal disorder is selected from Duchenne muscular dystrophy (DMD), myotonic dystrophy (DM), facioscapulohumeral muscular dystrophy (FSHD), and limb-girdle muscular dystrophy (LGMD).

203. The method of claim 199, wherein the musculoskeletal disorder is a type II collagenopathy.

204. The method of claim 199, wherein the musculoskeletal disorder is selected from achondrogenesis type II, hypochondrogenesis, Stickler Syndrome, and Czech Dysplasia.

205. The method of claim 199, wherein the musculoskeletal disorder is osteogenesis imperfecta (OI).

206. The method of claim 199, wherein the musculoskeletal disorder is an autoimmune musculoskeletal disease.

207. The method of claim 199, wherein the musculoskeletal disorder is an autoimmune musculoskeletal disease with polygenic susceptibility traits.

208. The method of claim 199, wherein the musculoskeletal disorder is a spondyloarthropathy (SA).

209. The method of claim 199, wherein the musculoskeletal disorder is ankylosing spondylitis.

210. The method of claim 199, wherein the musculoskeletal disorder is rheumatoid arthritis.

211. The method of claim 199, wherein the musculoskeletal disorder is autoimmune osteoarthritis.

212. The method of claim 199, wherein the musculoskeletal disorder is osteoarthritis (OA).

213. The method of claim 199, wherein the musculoskeletal disorder is Camurati-Engelmann disease.

214. The method of claim 199, wherein the musculoskeletal disorder is Sjogren's Syndrome.

215. The method of claim 199, wherein the musculoskeletal disorder is a mechanical musculoskeletal injury.

216. The method of claim 199, wherein the musculoskeletal disorder is selected from chronic muscle injury, torn tendon, post-traumatic osteoarthritis, and post-traumatic arthrofibrosis.

217. The method of any one of claims 142-216, wherein the at least one guide RNA or a nucleic acid encoding at least one guide RNA targets a mammalian TGFB1 gene.

218. The method of claim 217, wherein the mammalian TGFB1 gene is a canine, equine, or feline TGFB1 gene.

219. The method of claim 217, wherein the mammalian TGFB1 gene is a human TGFB1 gene.

220. The method of claim 219, wherein the at least one guide RNA comprises a spacer sequence selected from the group consisting of those sequences shown in FIGS. 1A-1D (SEQ ID NOS: 1-198).

221. The method of claim 219, wherein the at least one guide RNA comprises a spacer sequence selected from the group consisting of those sequences shown in FIG. 12A (SEQ ID NOS: 520-527).

222. The method of claim 219, wherein the at least one guide RNA comprises a spacer sequence of OHTG03 or OHTG04.

223. The method of any one of claims 142-216, wherein the at least one guide RNA or a nucleic acid encoding at least one guide RNA targets a mammalian TGFBR1 gene.

224. The method of claim 223, wherein the mammalian TGFBR1 gene is a canine, equine, or feline TGFBR1 gene.

225. The method of claim 223, wherein the mammalian TGFBR1 gene is a human TGFBR1 gene.

226. The method of claim 225, wherein the at least one guide RNA comprises a spacer sequence selected from the group consisting of those sequences shown in FIGS. 2A-2C (SEQ ID NOS: 199-320).

227. The method of claim 225, wherein the at least one guide RNA comprises a spacer sequence selected from the group consisting of those sequences shown in FIG. 12B (SEQ ID NOS: 528-552).

228. The method of claim 225, wherein the at least one guide RNA comprises a spacer sequence selected from the group consisting of those sequences shown in FIG. 5B.

229. The method of claim 225, wherein the at least one guide RNA comprises a spacer sequence of OHTIR04 or OHTIR08.

230. The method of any one of claims 142-216, wherein the at least one guide RNA or a nucleic acid encoding at least one guide RNA targets a mammalian TGFBR2 gene.

231. The method of claim 230, wherein the mammalian TGFBR1 gene is a canine, equine, or feline TGFBR1 gene.

232. The method of claim 230, wherein the mammalian TGFBR1 gene is a human TGFBR1 gene.

233. The method of claim 232, wherein the at least one guide RNA comprises a spacer sequence selected from the group consisting of those sequences shown in FIGS. 3A-3D (SEQ ID NOS: 321-519).

234. The method of claim 232, wherein the at least one guide RNA comprises a spacer sequence selected from the group consisting of those sequences shown in FIG. 12C (SEQ ID NOS: 553-604).

235. The method of claim 232, wherein the at least one guide RNA comprises a spacer sequence selected from the group consisting of those sequences shown in FIG. 6B.

236. The method of claim 232, wherein the at least one guide RNA comprises a spacer sequence of OHTIIR04.

237. The method of any one of claims 142-236, wherein the RNA-guided nuclease or a nucleic acid encoding an RNA-guided nuclease is the RNA-guided nuclease.

238. The method of any one of claims 142-236, wherein the RNA-guided nuclease or a nucleic acid encoding an RNA-guided nuclease is DNA encoding the RNA-guided nuclease.

239. The method of any one of claims 142-236, wherein the RNA-guided nuclease or a nucleic acid encoding an RNA-guided nuclease is mRNA encoding the RNA-guided nuclease.

240. The method of any one of claims 142-239, wherein the RNA-guided nuclease is a Cas protein.

241. The method of claim 240, wherein the Cas protein is a Cas9 protein.

242. The method of claim 241, wherein the Cas9 protein is an S. pyogenes Cas9 polypeptide.

243. The method of claim 241, wherein the Cas9 protein is selected from the group consisting of ESCas9, HFCas9, PECas9, and ARCas9.

244. The method of any one of claims 142-243, wherein the at least one guide RNA or a nucleic acid encoding at least one guide RNA is the at least one guide RNA.

245. The method of any one of claims 142-243, wherein the at least one guide RNA or a nucleic acid encoding at least one guide RNA is DNA encoding the at least one guide RNA.

246. The method of any one of claims 142-243, comprising a nucleic acid encoding both the RNA-guided nuclease and the at least one guide RNA.

247. The method of any one of claims 142-246, wherein the at least one guide RNA is a single guide RNA (sgRNA).

248. The method of any one of claims 142-247, wherein the composition comprises one or more viral vectors collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

249. The method of claim 248, wherein the one of more viral vectors comprise a recombinant virus selected from a retrovirus, an adenovirus, an adeno-associated virus, a lentivirus, and a herpes simplex virus-1.

250. The method of claim 248, wherein the one of more viral vectors comprise a recombinant adeno-associated virus (AAV).

251. The method of claim 250, wherein the recombinant AAV is of serotype 5 (AAV5).

252. The method of claim 250, wherein the recombinant AAV is of serotype 6 (AAV6).

253. The method of any one of claims 142-247, wherein the composition comprises one or more lipid nanoparticles (LNP) collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

254. The method of claim 253, wherein the one or more LNP comprises:
a first plurality of LNP encapsulating the RNA-guided nuclease or a nucleic acid encoding an RNA-guided nuclease; and
a second plurality of LNP encapsulating the at least one guide RNA or a nucleic acid encoding at least one guide RNA.

255. The method of claim 253, wherein the one or more LNP comprises a plurality of LNP encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

256. The method of any one of claims 253-255, wherein the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

257. The method of any one of claims 253-256, wherein the LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

258. The method of any one of claims 253-257, wherein the LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

259. The method of any one of claims 253-258, wherein the LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9, 11-dehydroergosterol, tomatidine, tomatine, a-tocopherol, and a mixture thereof.

260. The method of any one of claims 253-255, wherein the one or more LNP comprises an LP01 ionizable lipid, a neutral/helper lipid component, a steroid system component, and a pegylated lipid component.

261. The method of any one of claims 253-255, wherein the one or more LNP comprises an SM-102 ionizable lipid, a neutral/helper lipid component, a steroid system component, and a pegylated lipid component.

262. The method of any one of claims 253-255, wherein the one or more LNP comprises an ALC0315 ionizable lipid, a neutral/helper lipid component, a steroid system component, and a pegylated lipid component.

263. The method of any one of claims 260-262, wherein the neutral/helper lipid component comprises DSPC.

264. The method of claim 260-263, wherein the steroid system component comprises cholesterol.

265. The method of claim 260-264, wherein the steroid system component comprises a glucocorticoid steroid.

266. The method of claim 265, wherein the glucocorticoid steroid is dexamethasone.

267. The method of claim 260-266, wherein the pegylated lipid component comprises DMG-PEG.

268. The method of claim 267, wherein the DMG-PEG is DMG-PEG2000.

269. The method of any one of claims 260-268, wherein the one or more LNP comprises LP01, a neutral/helper lipid component, a steroid system component, and a pegylated lipid component at a molar ratio of (43-47): (7.5-10.5): (42-46): (1.5-2.5).

270. The method of any one of claims 260-268, wherein the one or more LNP comprises SM-102, a neutral/helper lipid component, a steroid system component, and a pegylated lipid component at a molar ratio of (48-52): (8.5-11.5): (36.5-40.5): (1-2).

271. The method of any one of claims 260-268, wherein the one or more LNP comprises ALC0315, a neutral/helper lipid component, a steroid system component, and a pegylated lipid component at a molar ratio of (48-52): (8.5-11.5): (36.5-40.5): (1-2).

272. The method of any one of claims 142-247, wherein the composition comprises one or more liposomes collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

273. The method of claim 272, wherein the one or more liposomes comprises:
a first plurality of liposomes encapsulating the RNA-guided nuclease or a nucleic acid encoding an RNA-guided nuclease; and
a second plurality of liposomes encapsulating the at least one guide RNA or a nucleic acid encoding at least one guide RNA.

274. The method of claim 272, wherein the one or more liposomes comprises a plurality of liposomes encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

275. The method of any one of claims 142-247, wherein the composition comprises one or more virus-like particles collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

276. The method of claim 275, wherein the one or more virus-like particles comprises:
a first plurality of virus-like particles encapsulating the RNA-guided nuclease or a nucleic acid encoding an RNA-guided nuclease; and
a second plurality of virus-like particles encapsulating the at least one guide RNA or a nucleic acid encoding at least one guide RNA.

277. The method of claim 275, wherein the one or more virus-like particles comprises a plurality of virus-like particles encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

278. The method of any one of claims 142-277, wherein the administering comprises parenteral administration.

279. The method of any one of claims 142-277, wherein the administering comprises intra-articular injection within a joint of the subject.

280. The method of any one of claims 142-277, wherein the administering comprises intradiscal injection.

281. The method of any one of claims 142-277, wherein the administering comprises peridiscal injection.

282. The method of any one of claims 142-277, wherein the administering comprises intravertebral injection.

283. The method of any one of claims 142-277, wherein the administering comprises administration to a cell or tissue of interest.

284. The method of any one of claims 142-277, wherein the administering is during surgery.

285. The method of claim 284, wherein the composition is painted onto the surgical site.

286. The method of claim 284, wherein the composition is sprayed onto the surgical site.

287. The method of claim 284, wherein the composition is coated on a bandage that is applied to the surgical site.

288. The method of claim 284, wherein the composition is coated on a stent implanted during surgery.

289. The method of claim 284, wherein the composition is dip coated onto an implant.
